(19) United States
(12) Patent Application Publication
(10) Pub. No.: US 2014/0162366 A1
(43) Pub. Date: Jun. 12, 2014

(54) GENERATION OF VASCULAR PROGENITOR CELLS

(71) Applicant: SALK INSTITUTE FOR BIOLOGICAL STUDIES, LA JOLLA, CA (US)

(72) Inventors: Juan Carlos Izpisua Belmonte, La Jolla, CA (US); Ignacio Sanchez-Martinez, San Diego, CA (US); Leo Kurian, San Diego, CA (US); Emmanuel Nivet, San Diego, CA (US)

(73) Assignee: SALK INSTITUTE FOR BIOLOGICAL STUDIES, LA JOLLA, CA (US)

(21) Appl. No.: 14/016,973

(22) Filed: Sep. 3, 2013

Related U.S. Application Data
(60) Provisional application No. 61/695,829, filed on Aug. 31, 2012.

(51) Int. Cl. C12N 15/85

(52) U.S. Cl. CPC C12N 15/85
USPC 435/467; 435/377

(57) ABSTRACT

Provided herein are, inter alia, methods of generating vascular progenitor cells from primary cells without reprogramming the primary cell into a pluripotent stem cell. The vascular progenitors provided herein may be used to form endothelial cells or smooth muscle cells. Further provided are isolated mutlitpotent cells useful to form vascular progenitor cells.

### CROSS-REFERENCE TO RELATED APPLICATIONS

This application claims benefit of priority to U.S. Provisional Patent Application No. 61/695,829, filed Aug. 31, 2012, which is incorporated herein by reference and for all purposes.

### STATEMENT AS TO RIGHTS TO INVENTIONS MADE UNDER FEDERALLY SPONSORED RESEARCH AND DEVELOPMENT

This invention was made with government support under R33MHO87925 awarded by the National Institutes of Health. The Government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 92150-007110US_ST25.TXT, created on Sep. 3, 2013, 54,739 bytes, machine format IBM-PC, MS-Windows operating system, is hereby incorporated by reference in its entirety for all purposes.

### BACKGROUND OF THE INVENTION

Somatic cell reprogramming has highlighted the plasticity of adult somatic cells as well as the possibility of generating any desired cell type in unlimited amounts. Three approaches for somatic cell reprogramming based on the forced expression of transcription factors (TFs) have been described (Sancho-Martinez, I., Nivet, E. & Izpisua Belmonte, J. C., J Mol Cell Biol 3, 327-329 (2011)). First, somatic cells can be reprogrammed into induced pluripotent stem cells (iPSCs), embryonic-like cells with the potential to generate all adult cell-types (Takahashi, K. & Yamanaka, S., Cell 126, 663-676 (2006)). Second, TFs defining or specifying target cell identity have proven successful for the direct lineage conversion of mouse and human cells into several cell types (Laiosa, C. V. et al., Immunity 25, 731-744 (2006); Ieda, M., et al., Cell 142, 375-386 (2010); Vierbuchen, T., et al., Nature 463, 1035-1041 (2010); Vierbuchen, T. & Wernig, M., Nat Biotechnol 29, 892-907 (2011)). Finally, the fact that reprogramming, or de-differentiation to iPSCs, proceeds in a step-wise manner suggests that the process can be stopped prior to the acquisition of an embryonic-like signature. Indeed, coupling a partially de-differentiated state to specific differentiation conditions has demonstrated a feasible alternative method to generate murine cardiac and neuronal cells (Efe, J. A., et al., Nat Cell Biol 13, 215-222 (2011); Kim, J., et al., Proc Natl Acad Sci USA, 108, 7838-7843 (2011); Sancho-Martinez, I. et al., Nat Cell Biol 14, 892-899 (2012); Papp, B. & Plath, K., Cell Res 21, 486-501 (2011); Plath, K. & Lowry, W. E., Nat Rev Genet. 12, 253-265 (2011)).

Here Applicants present a new and simple method for the efficient conversion of human fibroblasts into CD34+ progenitor cells with bi-potent differentiation potential. Applicants use a reprogramming strategy in which complete reprogramming to pluripotency is shortened or bypassed and the cells transition through a plastic intermediate state. This allows re-differentiation into CD34+ progenitor cells and subsequently to functional endothelial and smooth muscle cells. Applicants demonstrate for the first time that a reprogramming strategy involving partial de-differentiation is feasible in human cells. Altogether, described herein is a novel methodology for the reprogramming of somatic cells, which can be used as a complementary approach to direct lineage conversion and to full reprogramming to induced pluripotency for the generation of human cell types with clinical implications.

### BRIEF SUMMARY OF THE INVENTION

Provided herein are, inter alia, methods of generating vascular progenitor cells from primary cells without the requirement to reprogram the primary cell into a pluripotent cell. Surprisingly, Applicants show that vascular progenitor cells can be formed by deriving a multipotent cell from a primary cell. The mutlipotent cell can subsequently be differentiated into a vascular progenitor cell. Applicants have found that exposure of a primary cell (e.g., fibroblast) with reprogramming factors (e.g., Sox2, Oct4, Klf4) for a defined period of time (e.g., 8 days) results in the formation of a mutlipotent cell. The vascular progenitor cells provided herein including embodiments thereof, may further be cultured in the presence of a defined media composition, thereby allowing for the formation of smooth muscle cells and/or endothelial cells. The methods provided herein are particularly useful for clinical applications, for example, where a patient is in need of autologous vascular progenitor cells.

In one aspect, a method of generating a vascular progenitor cell is provided. The method includes transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby generating the vascular progenitor cell.

In another aspect, a method of generating a vascular progenitor cell is provided. The method includes culturing a multipotent cell in a solution including BMP4, bFGF, and VEGF, thereby generating the vascular progenitor cell.

In another aspect, an isolated multipotent cell is provided. The isolated multipotent cell is formed by a process including transfecting a primary cell with a non-integrative reprogramming nucleic acid, thereby forming a transfected primary cell. The transfected primary cell is allowed to grow, thereby forming the multipotent cell.

In another aspect, an isolated vascular progenitor cell is provided. The isolated vascular progenitor cell is formed by a process including transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby forming the vascular progenitor cell.

In another aspect, a method of generating an endothelial cell is provided. The method includes transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby generating the vascular progenitor cell. The vascular progenitor cell is cultured in endothelial cell growth media, thereby forming the endothelial cell.

In another aspect, an isolated endothelial cell formed according to the methods provided herein including embodiments thereof is provided.

In another aspect, a method of generating a smooth muscle cell is provided. The method includes transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby generating the vascular progenitor cell. The vascular progenitor cell is cultured in smooth muscle cell growth media, thereby forming the smooth muscle cell.

In another aspect, an isolated smooth muscle cell generated according to the methods provided herein including embodiments thereof is provided.

### BRIEF DESCRIPTION OF THE DRAWINGS

FIG. 1. Differentiation of hPSCs into mesodermal progenitor and endothelial cells. FIG. 1 (a) Scheme and Representative bright field pictures during the course of differentiation of human pluripotent stem cells towards CD34+ progenitor cells. FIG. 1 (b-e) Flow cytometry analysis of the mesoderm markers CD34 and CD31 over the differentiation course on HuES9 embryonic stem cells FIG. 1 (b), H1 embryonic stem cells FIG. 1 (c), two-factor cord blood-derived iPS cells (CBiPS) FIG. 1 (d) and four factor keratinocyte-derived iPS (KiPS) (e). Representative flow cytometry plots depicting a double CD34+ CD31+ population obtained after 8 days of PSC differentiation in the presence of Mesodermal Induction Media (MIM). Upper panel shows isotype controls. Lower panel shows specific CD34 and CD31 staining FIG. 1 (f). FIG. 1 (g) mRNA fold-change of pluripotency and mesodermal markers on KiPS. FIG. 1 (h) Fluorescence microscopy analysis showing the expression of indicated endothelial cell markers in KiPS derived Endothelial Cells)(KiPS^{Endo}). FIG. 1 (i) mRNA expression profile showing specific upregulation of endothelial markers in KiPS^{Endo}. FIG. 1 (j-k) Heat-map and representative clustering of hPSCs as compared to induced/differentiated endothelial (iECs) as well as a representative primary endothelial cell line (Human Umbilical Vein Endothelial Cells; HUVEC). On the left, genome-wide transcriptome analysis results demonstrate high similarities between PSC-differentiated and primary endothelial cells FIG. 1 (j). On the right, genome-wide methylation profiling representing the epigenetic changes occurring upon differentiation of PSCs into endothelial cells (iECs). Generated endothelial cells cluster closely to primary endothelial cells FIG. 1 (k). See Table 1 for specific gene-expression changes as summarized in the main Fig. panels. Error bars, s.d. Scale bars: 200 μm FIG. 1 (a), 50 μm FIG. 1 (h).

FIG. 2. Conversion of human fibroblasts into mesodermal progenitor and terminally differentiated endothelial cells by retroviral approaches. FIG. 2 (a) Schematic representation of the conversion process towards CD34+ progenitor cells and further differentiation into terminally differentiated endothelial cells. FIG. 2 (b) Representative flow cytometry plots demonstrating absent expression of pluripotency-associated markers upon plastic induction followed by Mesodermal Induction Media (MIM) differentiation. FIG. 2.(c) Flow cytometry analysis of CD34 expression after MIM induction in neonatal human fibroblasts in the presence of miR302-367 or the respective scramble controls. FIG. 2 (d) mRNA expression profiling of mesodermal genes upon the first phase of “plastic induction” (left panels); mRNA expression profiling of mesodermal genes upon “plastic induction” followed by MIM differentiation (right panels). Note the significant upregulation of all mesodermal and angioblast-related markers upon MIM exposure. FIG. 2 (e) mRNA expression profile showing specific upregulation of Endothelial Cell (EC) markers upon specific differentiation of sorted _{Fib}CD34+ cells into “converted” ECs. FIG. 2 (f) Fluorescence microscopy analysis showing the expression of the indicated endothelial markers in converted cells. FIG. 2 (g-h) Heat-map and representative clustering of the initial fibroblasts population as compare to converted _{Fib}CD34+ cells, endothelial cells (cECs) and a representative primary endothelial cell line (Human Umbilical Vein Endothelial Cells; HUVEC). On the left, genome-wide transcriptome analysis results demonstrating high similarities between converted and primary endothelial cells FIG. 2 (g). On the right, genome-wide methylation profiling representing the epigenetic changes occurring upon conversion into converted endothelial cells. Converted endothelial cells (cECs) cluster closely to primary endothelial cells FIG. 2 (h). See Table 1 for specific gene-expression changes as summarized in the main Fig. panels. Scale bars: 50 μm FIG. 2 (f). Error bars, s.d. *P<0.05.

FIG. 3. Conversion of human fibroblasts into mesodermal progenitor and terminally differentiated endothelial cells by non-integrative approaches. FIG. 3 (a) Schematic representation of the conversion process towards CD34+ progenitor cells and further differentiation into terminally differentiated endothelial cells. FIG. 3 (b) Representative flow cytometry plots demonstrating absent expression of pluripotency-associated markers upon plastic induction with non-integrative plasmids followed by Mesodermal Induction Media (MIM) differentiation. FIG. 3 (c) Representative flow cytometry analysis of CD34 expression before and after MIM differentiation in human fibroblasts (BJ) induced to a plastic state by the use of non-integrative approaches in the presence of miR302-367 or respective scramble controls. Upper panel shows isotype controls. Lower panel shows specific CD34. FIG. 3 (d) Representative flow cytometry quantification of BJ-converted VE-cadherin+ and endoglin+ endothelial cells derived in the presence of miR302/367 or respective scramble controls. FIG. 3 (e) mRNA expression profile showing specific upregulation of endothelial markers upon specific differentiation of sorted BJ _{Fib}CD34+ cells into “converted” endothelial cells. FIG. 3 (f) Fluorescence microscopy analysis showing the expression of the indicated endothelial markers in converted cells. FIG. 3 (g) Characterization of endothelial subtypes in BJ converted endothelial cells. Note the mixed expression of different endothelial subtype markers including arterial, venous and lymphatic upon conversion of fibroblasts. FIG. 3 (h) Representative pictures of endothelial cells derived by non-integrative approaches upon LDL uptake as compared to the respective controls (upper panels). LDL Mean Fluorescence Intensities of BJ-derived endothelial cells that were converted by non-integrative approaches (lower panels). Controls represent converted endothelial cells in the presence of Alexa Fluor 488 in order to measure unspecific fluorescence background. FIG. 3 (i) BJ-derived endothelial cells converted by non-integrative approaches spontaneously formed capillary-like structures in vitro. See Table 1 for specific gene-expression changes as summarized in the main Fig. panels. Scale bars: 50 μm FIG. 3 (f); 100 μm FIG. 3 (h); 200 μm FIG. 3 (i). Error bars, s.d. *P<0.05.

FIG. 4. Generated Endothelial cells demonstrate functionality in vivo. FIG. 4 (a) 17 days after injection, matrigel plugs were extracted and processed for analyses. Pictures show increased blood circulation through the extracted plugs, thus demonstrating connection with the pre-existing vasculature (anastomosis) in vivo. FIGS. 4 (b,c) Representative pictures of HuES9− (left panels) and KiPS− (right) derived endothelial cells showing the identification of human cells by in situ hybridization on ALU+ sequences (dark dot), anti-human CD31 staining and Ulex Lectin rhodamine staining Note the presence of circulating red blood cells through the vessel-graft. FIG. 4 (d) Endothelial cells derived by non-integrative approaches-mediated conversion of human fibroblasts demonstrate anastomosis in vivo. Human specific CD31 antibody demonstrates the presence of converted endothelial cells. Co-localization with specific Human Nuclear Antigen staining demonstrates that the generated vessels are derived from the injected converted human endothelial cells. FIG. 4 (e) Representative high magnification picture demonstrating connection to the pre-existing vasculature upon injection of converted endothelial cells generated by non-integrative approaches. White arrows indicate the presence of circulating red blood cells. Scale bars: 5 μm FIGS. 4 (b,c); 50 μm FIGS. 4 (d,e).

FIG. 5. Detailed analysis of the different pscCD34+ populations obtained by day 8 of differentiation in the presence of MIM. FIG. 5 (a) Representative flow cytometry gating strategy. Briefly, the living population was gated and displayed in terms of CD34 and CD31 expression on contour plots (upper panel). Each different population observed was further gated and analyzed in terms of their KDR and c-Kit expression (lower panels). All percentages shown in the manuscript are referred to the initial living population representing the total number of cells after isotype background subtraction. FIG. 5 (b-e) Percentages of positive cells for each different population observed in different Embryonic Stem Cells (ESC) and induced Pluripotent Stem Cell (iPSC) lines. Note that most of the CD31+ cells are comprised inside a CD34+ CD31+KDR+c-Kit+ quadruple positive population. The percentages of CD34+ CD31− and CD34+ CD31+ represent the total number of CD34+ cells regardless their KDR and c-Kit expression as represented in FIG. 1 to allow visual comparison. Detailed analysis of KDR and c-Kit expression of these populations is also shown. Error bars, s.d.

FIG. 6. mRNA expression profiling of hPSC lines upon MIM induction. FIG. 6 (a-d) Mesodermal marker expression on the embryonic stem cell line HuES9 FIG. 6 (a); H1 FIG. 6 (b); Cord-Blood derived iPSCs FIG. 6 (c) and KiPSCs FIG. 6 (d). Error bars, s.d.

FIG. 7. hPSC-derived-CD34+ progenitors show endothelial differentiation potential. FIG. 7 (a) Sorted CD34+ progenitors under endothelial differentiation conditions showed efficient and robust generation of endothelial-like cells as measured by flow cytometry analysis of VE-cadherin and endoglin expression. FIG. 7 (b) Representative contour plots showing differentiated VE-cadherin+endoglin+ cells. FIGS. 7 (c,e) Fluorescence microscopy analysis showing the expression of indicated endothelial cell markers in CBiPS FIG. 7 (c); and H1 FIG. 7 (e). FIG. 7 (d) mRNA expression profile showing specific upregulation of endothelial cell markers in CBiPS FIG. 7 (d). Error bars, s.d. Scale bars: 100 μm FIGS. 7 (c,e).

FIG. 8. hPSC-derived-CD34+ progenitors show smooth muscle differentiation potential. FIG. 8 (a) mRNA profiling of _{PSC}CD34+ cells differentiated to smooth muscle cells demonstrates specific smooth muscle marker upregulation whereas endothelial markers, serving as internal controls for specificity, remained unchanged. On the left, representative marker expression of smooth muscle differentiated H1 ESCs; On the right, representative marker expression of smooth muscle differentiated Cord-Blood iPSCs; FIG. 8 (b,c) On the left, mRNA profiling of MIM-differentiated HuES9 ESC-derived pscCD34+ cells FIG. 8 (b) and KiPS-derived _{PSC}CD34+ FIG. 8 (c) indicating mixed mesodermal origins. On the middle, _{PSC}CD34+ cells differentiated to smooth muscle cells demonstrating specific smooth muscle markers. On the right, analysis of the specific smooth muscle subtypes generated indicates the presence of different smooth muscle sub-populations. FIG. 8 (d) Representative immunostaining of sorted pscCD34+ cells differentiated into calponin+ and α-smooth muscle actin+ smooth muscle cells; nuclei were counterstained with Hoechst. FIG. 8 (e) Single-cell differentiation assays demonstrating the multipotent nature of MIM-induced CD34+ progenitor cells. Error bars, s.d. Scale bars: 50 μm FIG. 8 (f).

FIG. 9. Genetic and Epigenetic analyses on PSC-differentiated and Fibroblasts-converted endothelial cells. FIG. 9 (a) Venn diagram analysis depicting common methylation changes during the directed differentiation of PSCs. All PSC (ESCs/iPSCs) groups as well as their respectively differentiated endothelial cells (ESC^{iEC}/iPSC^{iEC}) and primary Human Umbilical Vein Endothelial Cells (PriEC) were compared. FIG. 9 (b) Venn diagram analysis depicting common methylation changes during the indirect conversion of human fibroblasts. Fibroblast groups (Fibro) were compared to primary Human Umbilical Vein Endothelial Cells (PriEC) as well as their respectively converted endothelial cells (cEC^{4F}/cEc^{4F/miRs}). FIG. 9 (c) Representative heat-map depicting methylation changes and clusters upon comparison of all different initial populations and their respectively derived endothelial cells including those derived by directed differentiation of PSCs as well as those derived by conversion of human fibroblasts and primary Human Umbilical Vein Endothelial Cells. FIG. 9 (d) Venn diagram analysis depicting common differentially regulated mRNAs during the directed differentiation of PSCs. All PSC (ESCs/iPSCs) groups as well as their respectively differentiated endothelial cells (ESC^{iEC}/iPSC^{iEC}) and primary Human Umbilical Vein Endothelial Cells (PriEC) were compared. FIG. 9 (e) Venn diagram analysis depicting common differentially regulated mRNAs during the indirect conversion of human fibroblasts. Fibroblast groups (Fibro) were compared to primary Human Umbilical Vein Endothelial Cells (PriEC) as well as their respectively converted endothelial cells (cEC^{4F}/cEc^{4F/miRs}). FIG. 9 (f) Representative heat-map depicting mRNA changes and clusters upon comparison of all different initial populations and their respectively derived endothelial cells including those derived by directed differentiation of PSCs as well as those derived by conversion of human fibroblasts and primary Human Umbilical Vein Endothelial Cells. See Table 1 for specific gene and methylation changes as summarized in the Fig. panels.

FIG. 10. Genetic and Epigenetic analyses on PSC-differentiated and Fibroblasts-converted smooth muscle cells. FIG. 10 (a) Venn diagram analysis depicting common methylation changes during the directed differentiation of PSCs. All PSC (ESCs/iPSCs) groups as well as their respectively differentiated endothelial cells (ESe^{iSMC}/iPSC^{iSMC}) and human Arterial Smooth Muscle Cells (PriSMC) were compared. FIG. 10 (b) Venn diagram analysis depicting common methylation changes during the indirect conversion of human fibroblasts. Fibroblast groups (Fibro) were compared to primary human Arterial Smooth Muscle Cells (PriSMC) as well as their respectively converted endothelial cells (cSMC^{4F}/csmc^{4F/miRs}). FIG. 10 (c) Representative heat-map depicting methylation changes and clusters upon comparison of all different initial populations and their respectively derived smooth muscle cells including those derived by directed differentiation of PSCs as well as those derived by conversion of human fibroblasts and primary human Arterial Smooth Muscle Cells. FIG. 10 (d) Venn diagram analysis depicting common differentially regulated mRNAs during the directed differentiation of PSCs. All PSC (ESCs/iPSCs) groups as well as their respectively differentiated endothelial cells (ESC^{iSMC}/iPSC^{iSMC}) and human Arterial Smooth Muscle Cells (PriSMC) were compared. FIG. 10 (e) Venn diagram analysis depicting common differentially regulated mRNAs during the indirect conversion of human fibroblasts. Fibroblast groups (Fibro) were compared to primary human Arterial Smooth Muscle Cells (PriSMC) as well as their respectively converted endothelial cells (cSMC^{4F}/cSMC^{4F/miRs}). FIG. 10 (f) Representative heat-map depicting mRNA changes and clusters upon comparison of all different initial populations and their respectively derived smooth muscle cells including those derived by directed differentiation of PSCs as well as those derived by conversion of human fibroblasts and primary human Arterial Smooth Muscle Cells. See Table 1 for specific gene and methylation changes as summarized in the Fig. panels.

FIG. 11. Conversion of neonatal and adult human fibroblasts into terminally differentiated endothelial cells by viral approaches. FIG. 11 (a) Representative flow cytometry quantification of adult Human Dermal Fibroblasts (HDF)-converted VE-cadherin+ and endoglin+ endothelial cells. FIG. 11 (b) Fluorescence microscopy analysis showing the expression of the VE-cadherin, endoglin and vWF endothelial markers in the HDF-converted cells; nulcei were counterstained with Hoechst. FIG. 11 (c) HDF-derived endothelial cells spontaneously formed capillary-like structures in vitro (upper panel). LDL Mean Fluorescence Intensities (lower panel) of HDF-derived endothelial cells. Controls represent converted endothelial cells in the presence of Alexa Fluor 488 in order to measure unspecific fluorescence background. FIG. 11 (d) Representative flow cytometry quantification of neonatal Human BJ Fibroblasts (BJ)-converted VE-cadherin+ and endoglin+ endothelial cells. FIG. 11 (e) Fluorescence microscopy analysis showing the expression of the VE-cadherin, endoglin and vWF endothelial markers in the BJ-converted cells; nulcei were counterstained with Hoechst. FIG. 11 (f) BJ-derived endothelial cells spontaneously formed capillary-like structures in vitro (upper panel). LDL Mean Fluorescence Intensities (lower panels) of BJ-derived endothelial cells. Controls represent converted endothelial cells in the presence of Alexa Fluor 488 in order to measure unspecific fluorescence background. Error bars, s.d. Scale bars: 50 μm FIGS. 11 (b,e); 500 μm FIG. 11 (c,f).

FIG. 12. Generated endothelial cells represent a mixed population of different endothelial sub-types. FIG. 12 (a-c) mRNA expression profiling of endothelial sub-type specific genes demonstrating the upregulation of arterial, venous and lymphatic markers upon endothelial differentiation of MIM-induced CD34+ cells from PSCs FIG. 12 (a); and upon conversion of neonatal human fibroblasts FIG. 12 (b); as well as adult human fibroblasts FIG. 12 (c). Error bars, s.d. *P-value<0.05.

FIG. 13. Neonatal and Adult Fibroblast-derived-CD34+ progenitors show smooth muscle differentiation potential. FIGS. 13 (a,b) mRNA profiling of the generated _{Fib}CD34+ cells as well as the corresponding differentiated to smooth muscle cells from adult human fibroblasts (HDF) derived by viral approaches FIG. 13 (a) and neonatal fibroblasts (BJ) derived by non-integrative approaches FIG. 13 (b). On the left, representative marker expression of intermediate populations indicating mixed mesodermal origins. On the middle, _{Fib}CD34+ cells differentiated to smooth muscle cells demonstrating specific smooth muscle markers; On the right, mRNA profiling of MIM-differentiated adult _{Fib}CD34+-derived smooth muscle cells indicating the presence of different smooth muscle sub-populations FIG. 13 (c) Fluorescence microscopy analysis demonstrating the expression of the indicated smooth muscle markers in two neonatal human fibroblast lines (HFF, BJ) and adult human fibroblasts (HDF) derived smooth muscle cells converted by both, viral and non-integrative approaches. Error bars, s.d. Scale bars: 50 μm FIG. 13 (c). *P-value<0.05.

FIG. 14. Conversion of human fibroblasts does not require sustained reprogramming factor or pluripotent-marker expression. FIGS. 14 (a,b,c) mRNA expression profiling of pluripotency genes demonstrating rapid downregulation during the course of conversion relative to undifferentiated human fibroblasts. FIG. 14 (d) Expression of pluripotency factors during the first phase of conversion (“plastic induction”) did not result in pluripotent marker expression at the protein level. FIG. 14 (e-h) Copy number and mRNA levels of the different pluripotency-related genes demonstrating random integration in virus-converted endothelial cells FIG. 14 (e) as compared to absent mRNA expression of exogenous genes FIG. 14 (f) due to the rapid clearing observed when non-integrative approaches were utilized FIG. 14 (g). FIGS. 14 (h,i) Ten weeks post-transplantation into the testis, differentiated endothelial cells did not give rise to teratoma formation FIG. 14 (h) and their endothelial identity remained unchanged FIG. 14 (i), as compared to undifferentiated PSC controls (number of animals per group: n=4 for PSC differentiated cells and n=4 for endothelial cells converted by non-integrative approaches). Error bars, s.d. Scale bars: 500 μm FIG. 14 (h); 20 μm FIG. 14 (i). *P-value<0.05.

FIG. 15. PSC- and Fibroblasts-derived smooth muscle cells exhibit physiological traits characteristic of mature smooth muscle cells. FIG. 15 (a) Calcium response to 100 μM carbachol stimulation. All cell types presented calcium transients characteristic of muscarinic receptor stimulation, with a fast initial response initiated by extracellular calcium entry and a secondary response induced by IP₃-mediated intracellular calcium mobilization. FIG. 15 (b) Carbachol provokes cell body contraction in PSC-differentiated and Fibroblast-converted smooth muscle cells. FIG. 15 (c) Representative plot demonstrating spontaneous Calcium transients in adult human fibroblast (HDF)-derived smooth muscle cells. The derived smooth muscle cells exhibit spontaneous calcium activity coupled to contraction and relaxation events typical of mature SMCs. A minimum of 20 different cells per experiment in at least two different biological experiments has been recorded. Error bars, s.e.m. *P-value<0.05.

FIG. 16. Generated Endothelial cells demonstrate functionality in vitro. FIG. 16 (a-c) On the left, LDL Mean Fluorescence Intensities of KiPSC—FIG. 16 (a), CBiPS—FIG. 16 (b) and H1-derived endothelial cells FIG. 16 (c) as compared to the respective negative controls. Controls represent converted endothelial cells in the presence of Alexa Fluor 488 in order to measure unspecific fluorescence background. On the right, spontaneous formation of capillary-like structures in vitro in the different PSC-differentiated endothelial cells. Error bars, s.d. Scale bars: 500 μm.

### DETAILED DESCRIPTION OF THE INVENTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein might be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or “percent identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “transfection” or “transfecting” is defined as a process of introducing nucleic acid molecules to a cell by non-viral or viral-based methods. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods of transfection any useful viral vector may be used in the methods described herein. Examples for viral vectors include, but are not limited to retroviral, adenoviral, lentiviral and adeno-associated viral vectors.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently (e.g., when a non-integrative nucleic acid is used) or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” or “non-integrative” refers to the extra-chromosomal state of a nucleic acid in a cell. Episomal or non-integrative nucleic acid molecules are not part of the chromosomal DNA and replicate independently thereof. A non-integrative nucleic acid as used herein may be a plasmid or a viral vector. A non-integrative reprogramming nucleic acid as used herein refers to a plasmid or viral vector that does not form part of the chromosomal DNA and encodes for reprogramming polypeptides. Examples of reprogramming polypeptides are without limitation OCT4, SOX2, KLF4, Nanog, and c-Myc.

The term “Yamanaka factors” refers to Oct3/4, Sox2, Klf4, and c-Myc, which factors are highly expressed in embryonic stem (ES) cells. Yamanaka factors can induce pluripotency in somatic cells from a variety of species, e.g., mouse and human somatic cells. See e.g., Yamanaka, 2009, Cell 137: 13-17.

A “KLF4 protein” as referred to herein includes any of the naturally-occurring forms of the KLF4 transcription factor, or variants thereof that maintain KLF4 transcription factor activity (e.g. within at least 50%, 80%, 90%, 95%, 96%, 97%, 98%, 99% or 100% activity compared to KLF4). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLF4 polypeptide. In other aspects, the KLF4 protein is the protein as identified by the NCBI reference gi:194248077 (SEQ ID NO:1) or a variant having substantial identity thereto.

An “OCT4 protein” as referred to herein includes any of the naturally-occurring forms of the Octomer 4 transcription factor, or variants thereof that maintain Oct4 transcription factor activity (e.g. within at least 50%, 80%, 90%, 95%, 96%, 97%, 98%, 99% or 100% activity compared to Oct4). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Oct4 polypeptide. In other aspects, the Oct4 protein is the protein as identified by the NCBI reference gi:42560248 (SEQ ID NO:2) corresponding to isoform 1, gi:116235491 (SEQ ID NO:3) and gi:291167755 (SEQ ID NO:4) corresponding to isoform 2, or a variant having substantial identity thereto.

A “SOX2 protein” as referred to herein includes any of the naturally-occurring forms of the SOX2 transcription factor, or variants thereof that maintain SOX2 transcription factor activity (e.g. at least 50%, 80%, 90%, 95%, 96%, 97%, 98%, 99% or 100% activity compared to Sox2). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion, e.g., the DNA-binding region) compared to a naturally occurring Sox2 polypeptide. In embodiments, the SOX2 protein is the protein as identified by the NCBI reference gi:28195386 (SEQ ID NO:5) or a variant having substantial identity thereto.

A “cMYC protein” as referred to herein includes any of the naturally-occurring forms of the cMyc transcription factor, or variants thereof that maintain cMyc transcription factor activity (e.g. within at least 50%, 80%, 90%, 95%, 96%, 97%, 98%, 99% or 100% activity compared to cMyc). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cMyc polypeptide. In other aspects, the cMyc protein is the protein as identified by the NCBI reference gi:71774083 (SEQ ID NO:6), or a variant having substantial identity thereto.

A “TRA1-81 protein” as referred to herein includes any of the naturally-occurring forms of the TRA1-81 cell surface glycoprotein, or variants thereof that maintain TRA1-81 protein activity (e.g. within at least 50%, 80%, 90%, 95%, 96%, 97%, 98%, 99% or 100% activity compared to TRA1-81 protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRA1-81 protein. In embodiments, the TRA1-81 protein is a protein identified by Uniprot reference 000592 (SEQ ID NO:129), or a variant having substantial identity thereto.

A “TRA1-60 protein” as referred to herein includes any of the naturally-occurring forms of the TRA1-60 cell surface glycoprotein, or variants thereof that maintain TRA1-60 protein activity (e.g. within at least 50%, 80%, 90%, 95%, 96%, 97%, 98%, 99% or 100% activity compared to TRA1-60 protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRA1-60 protein. In other aspects, the TRA1-60 protein bound by a TRA1-60 antibody.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. The term does not imply that all cells in the culture survive or grow or divide, as some may naturally senesce, etc. Cells are typically cultured in media, which can be changed during the course of the culture.

The terms “media” and “culture solution” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

As used herein, “conditions to allow growth” in culture and the like refers to conditions of temperature (typically at about 37° C. for mammalian cells), humidity, CO₂ (typically around 5%), in appropriate media (including salts, buffer, serum), such that the cells are able to undergo cell division or at least maintain viability for at least 24 hours, preferably longer (e.g., for days, weeks or months).

Suitable culture conditions are described herein, and can include standard tissue culture conditions. For example, progenitor cells or somatic (primary) cells can be cultured in a buffered media that includes amino acids, nutrients, growth factors (e.g., BMP4, bFGF, VEGF), etc., as will be understood in the art. In embodiments, the culture of progenitor cells includes feeder cells (e.g., fibroblasts), while in others, the culture is devoid of feeder cells. Cell culture conditions are described in more detail, e.g., in Picot, Human Cell Culture Protocols (Methods in Molecular Medicine) 2010 ed. and Davis, Basic Cell Culture 2002 ed.

Culture conditions that support differentiation of a multipotent cell as provided herein to a smooth muscle cell or an endothelial cell include BMP4, bFGF and VEGF. BMP4 (Bone morphogenetic protein 4) as provided herein refers to

“BMP4” as referred to herein refers to “Bone morphogenetic protein 4” and includes any of the naturally-occurring forms of the BMP4 growth factor, or variants thereof that maintain BMP4 growth factor activity (e.g. within at least 50%, 80%, 90%, 95%, 96%, 97%, 98%, 99% or 100% activity compared to BMP4). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BMP4 polypeptide. In other aspects, the BMP4 protein is the protein as identified by the NCBI reference G1:157276593 (SEQ ID NO:130) or a variant having substantial identity thereto.

“bFGF” as referred to herein refers to “Basic Fibroblast Growth Factor” and includes any of the naturally-occurring forms of the bFGF growth factor, or variants thereof that maintain bFGF growth factor activity (e.g. within at least 50%, 80%, 90%, 95%, 96%, 97%, 98%, 99% or 100% activity compared to bFGF). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring bFGF polypeptide. In other aspects, the bFGF protein is the protein as identified by the NCBI reference G1:153285461 (SEQ ID NO:131) or a variant having substantial identity thereto.

“VEGF” as referred to herein refers to “Vascular Endothelial Growth Factor” and includes any of the naturally-occurring forms of the VEGF growth factor, or variants thereof that maintain VEGF growth factor activity (e.g. within at least 50%, 80%, 90%, 95%, 96%, 97%, 98%, 99% or 100% activity compared to VEGF). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VEGF polypeptide. In other aspects, the VEGF protein is the protein as identified by the Uniprot reference P15692 (SEQ ID NO:132) or a variant having substantial identity thereto.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

The term “isolated,” when referring to a cell or molecule (e.g., nucleic acid or protein), indicates that the cell or molecule is or has been separated from its natural environment. For example, an isolated cell can be removed from its host individual, but still exist in culture with other cells, or be reintroduced into its host individual.

The term “feeder-free,” refers to the absence of feeder cells. The term “feeder cell” is known in the art, and includes all cells used to support the propagation of stem cells, e.g., during the process of reprogramming. Feeder cells can be irradiated prior to being co-cultured with the stem cells in order to avoid the feeder cells outgrowing the stem cells. Feeder cells provide a layer physical support for attachment, and produce growth factors and extracellular matrix proteins that support cells. Examples of feeder cells include fibroblasts (e.g., embryonic fibroblasts), splenocytes, macrophages and thymocytes.

A “somatic cell” is a cell forming the body of an organism. Somatic cells include cells making up organs, skin, blood, bones and connective tissue in an organism, but not germ line cells or stem cells.

A “stem cell” is a cell characterized by the ability of self-renewal through mitotic cell division and the potential to differentiate into a tissue or an organ. Among mammalian stem cells, embryonic and somatic stem cells may be distinguished. Embryonic stem cells may reside in the blastocyst and give rise to embryonic tissues, whereas somatic stem cells may reside in adult tissues for the purpose of tissue regeneration and repair.

A “pluripotent stem cell” refers to a cell having the ability of self-renewal through mitotic cell division and the potential to differentiate into cell types of all three germ layers (i.e. mesenchyme, endoderm and ectoderm).

A “non-pluripotent cell” refers to a cell of lesser potency to self-renew and differentiate than a pluripotent stem cell. Examples of non-pluripotent cells are without limitation somatic stem cells, tissue specific progenitor cells, primary or secondary cells. In some embodiments, the non-pluripotent stem cell is a multipotent cell. A “multipotent” cell as referred to herein is a cell exhibiting lesser self-renewal capacity than a pluripotent stem cell but more self-renewal capacity than a primary cell. A multipotent cell may not be able to give rise to cells of all three germ layers and is committed to differentiate into a specific organ or tissue (e.g. blood cells, smooth muscle cells, endothelial cells). In some embodiments, a multipotent cell is a somatic stem cell. Without limitation, a somatic stem cell can be a hematopoietic stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells (e.g., fibroblasts), bone cells, blood cells, cells of internal organs and cells of connective tissue. A secondary cell is derived from a primary cell and has been immortalized for long-lived in vitro cell culture.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers (pluripotency markers) are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics (e.g., embryonic stem cell colony formation, teratoma formation).

The term “reprogramming” refers to the process of dedifferentiating a non-pluripotent cell into a cell exhibiting pluripotent stem cell characteristics.

Where appropriate the expanding transfected derived stem cell may be subjected to a process of selection. A process of selection may include a selection marker introduced into an induced pluripotent stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected induced pluripotent stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection marker, a toxin may be converted to a non-toxin, which no longer inhibits expansion and causes cell death of a transfected induced pluripotent stem cell. Upon exposure to a toxin, a cell lacking a selection marker may be eliminated and thereby precluded from expansion.

Identification of the induced pluripotent stem cell may include, but is not limited to the evaluation of afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

The terms “vascular progenitor cell,” “mesodermal progenitor cell,” or “angioblast-like progenitor” refer to a bipotent progenitor cell. A bipotent progenitor cell as provided herein is a progenitor cell, can give rise to two cell types. The bipotent vascular progenitor cell provided herein has the ability to form two mesodermal lineage cell types, endothelial cells and smooth muscle cells. Vascular progenitor cells can be identified by expression of characteristic markers, e.g., CD34. In embodiments, the vascular progenitor cell is CD34+. In embodiments, the vascular progenitor also expresses at least one cell surface marker selected from CD31 (PECAM-1), c-Kit, and KDR. In embodiments, the vascular progenitor cell does not express CD133.

“Self renewal” refers to the ability of a cell to divide and generate at least one daughter cell with the self-renewing characteristics of the parent cell. The second daughter cell may commit to a particular differentiation pathway. For example, a self-renewing hematopoietic stem cell can divide and form one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Non-self renewing cells refers to cells that undergo cell division to produce daughter cells, neither of which have the differentiation potential of the parent cell type, but instead generates differentiated daughter cells.

“Allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

“Autologous” refers to deriving from or originating in the same subject or patient. An “autologous transplant” refers to collection and retransplant of a subject's own cells or organs.

As defined herein, the term “inhibition”, “inhibit”, “inhibiting” and the like in reference to a protein-inhibitor interaction means negatively affecting (e.g. decreasing) the activity or function of the protein (e.g. decreasing expression of p53) relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway (e.g. reduction of a pathway involving p53 activity). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. p53). In embodiments, inhibition refers to inhibition of p53.

The terms “subject,” “patient,” “individual,” etc. are not intended to be limiting and can be generally interchanged. That is, an individual described as a “patient” does not necessarily have a given disease or deficiency, but may be merely seeking medical advice.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “dose” and “dosage” are used interchangeably herein. A dose refers to the amount of active ingredient (e.g., EC, SMC, or hematopoietic cell) given to an individual at each administration. For the present invention, the dose can be expressed, e.g., in cells/kg of the individual receiving the treatment, or cells/volume of the pharmaceutical solution administered. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical, and depends on the route of administration. For the present invention, the dosage form is typically in a liquid or semi-liquid form, such as a saline solution for injection.

As used herein, the terms “treat” and “prevent” are not intended to be absolute terms. Treatment can refer to any reduction in the frequency or severity of symptoms, amelioration of symptoms, improvement in patient comfort and/or function, etc. The effect of treatment can be compared to an individual or pool of individuals not receiving a given treatment, or to the same patient prior to, or after cessation of, treatment.

As used herein, the terms “pharmaceutically” acceptable is used synonymously with physiologically acceptable and pharmacologically acceptable. A pharmaceutical composition will generally comprise agents for buffering and preservation in storage, and can include buffers and carriers for appropriate delivery, depending on the route of administration.

The term “prevent” refers to a decrease in the occurrence of symptoms of a cell deficiency in a patient. As indicated above, the prevention may be complete (no detectable symptoms) or partial, such that fewer symptoms are observed than would likely occur absent treatment.

The term “therapeutically effective amount,” as used herein, refers to that amount of the therapeutic agent sufficient to ameliorate the disorder. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

### A. METHODS OF GENERATING MULTIPOTENT CELLS AND VASCULAR PROGENITOR CELLS

Provided herein are, inter alia, methods of generating vascular progenitor cell from primary cells (e.g., fibroblasts). Primary cells for use in the present methods can be obtained from any source, e.g., skin, from an individual or group of individuals, or from a cell line, e.g., a fibroblast cell line. In embodiments, the primary cell is a human fibroblast. In embodiments, the primary cell is obtained from the same individual that is intended to receive a transplant of vascular progenitor cells formed by the methods provided herein including embodiments thereof. In embodiments, the primary cell is obtained from the same individual that is intended to receive a transplant of smooth muscle cells and/or endothelial cells formed from the vascular progenitor cells according to the methods provided herein including embodiments thereof. That is, the primary cells, and the cells generated therefrom, are autologous to the donor/recipient. In embodiments, the primary cell donor(s) is different than the recipient, such that the transplanted cells will be allogeneic to the recipient.

Primary cells can be from any mammal, e.g., human, non-human primate, mouse, rat, rabbit, dog, cat, horse, cow, pig, sheep, goat, etc. Typically, the source of the cell is determined based on the intended use. For example, for transplant, primary cells from the same species can be used for the generation of the cell type to be transplanted. In embodiments, however, a xenotransplant can be carried out such that primary cells from a different species are used to form smooth muscle cells and/or endothelial cells to be transplanted into the recipient.

The primary cell may be transfected with a non-integrative reprograming nucleic acid to form a transfected primary cell. The non-integrative reprograming nucleic acid may encode one or more reprogramming factors (e.g., one or more Yamanaka factors). In embodiments, a plurality of non-integrative reprograming nucleic acids is transfected, wherein each non-integrative reprograming nucleic acid encodes a single reprogramming factor. In embodiments, the non-integrative reprograming nucleic acid encodes a Sox-2 polypeptide, a Oct-4 polypeptide, a Klf-4 polypeptide, a cMyc polypeptide, a Lin28 polypeptide or a p53 siRNA. In embodiments, the non-integrative reprograming nucleic acid encodes a Sox-2 polypeptide, a Oct-4 polypeptide, a Klf-4 polypeptide, a cMyc polypeptide, a Lin28 polypeptide and a p53 siRNA. In embodiments, the non-integrative reprograming nucleic acid includes a first non-integrative reprograming nucleic acid molecule and a second non-integrative reprograming nucleic acid molecule. In embodiments, the first non-integrative reprograming nucleic acid molecule encodes a Sox-2 polypeptide, a Oct-4 polypeptide, a Klf-4 polypeptide, a cMyc polypeptide, or a Lin28 polypeptide. In embodiments, the first non-integrative reprograming nucleic acid molecule encodes a Sox-2 polypeptide, a Oct-4 polypeptide, a Klf-4 polypeptide, a cMyc polypeptide, and a Lin28 polypeptide. In further embodiments, the second non-integrative reprograming nucleic acid molecule encodes a p53 siRNA.

A “siRNA” or “small interfering RNA” is a a short (usually 21-bp) double-stranded ribonucleotide sequence with phosphorylated 5′ ends and hydroxylated 3′ ends with two overhanging nucleotides, which can be used to silence gene expression. After processing by cellular factors the siRNA interacts with a complementary RNA (e.g., p53 RNA) thereby interfering with the expression of the complementary RNA. Any siRNA capable of inhibiting p53 expression in a cell is contemplated for the methods provided herein.

Provided herein are in vitro generated vascular progenitor cells derived from primary cell-derived multipotent cells. These vascular progenitor cells can be generated in vitro in a matter of days in the absence of potential contaminants, such as feeder cells. Thus, in embodiments, the allowing to grow in step (ii) is performed in the absence of feeder cells. The transfected primary cell may be allowed to grow for less than 10 days, but typically more than 2, 3, 4, 5, or 6. Thus, in embodiments, the allowing to grow in step (ii) is performed for less than 10 days. In embodiments, the allowing to grow in step (ii) is performed for less than 9 days. In other embodiments, the allowing to grow in step (ii) is performed for less than 8 days. In embodiments, the allowing to grow in step (ii) is performed for about 7 days. In embodiments, the allowing to grow in step (ii) is performed for about 8 days. In embodiments, the allowing to grow in step (ii) is performed for about 9 days. One of skill will understand that, during the culturing period, the initial transfected primary cell or population of transfected primary cells can divide and/or change character as the transfected primary cell gives rise to a multipotent cell. Indeed, the method is typically carried out with a plurality of transfected primary cells to form a population of cells that includes, over time, increasing numbers of multipotent cells. In embodiments, the population of cells comprises 30-80% multipotent cells, e.g., 35, 40, 45, 50, 60, 65, 70, 75%, or higher percentage multipotent cells.

The methods provided herein provide for a fast and efficient way of generating vascular progenitor cells from primary cells without the formation of pluripotent stem cells. The primary cell (e.g., fibroblast) forms a multipotent cell, which subsequently forms a vascular progenitor cell. The multipotent cell as provided herein is characterized by the lack of expression of pluripotency stem cell characteristics (e.g., expression of pluripotency markers such as SSEA-3, SSEA-4, TRA-1-60, or TRA-1-81; cell morphologic features e.g., formation of embryonic stem cell colonies). Thus, in embodiments, the multipotent cell lacks pluripotency stem cell characteristics. In embodiments, the multipotent cell does not express a pluripotency marker. In embodiments, the pluripotency marker is a TRA1-81 polypeptide or a TRA1-60 polypeptide. In embodiments, the multipotent cell is not capable of forming an embryonic stem cell colony. In embodiments, the multipotent cell is not capable of teratoma formation.

Once the multipotent cell is formed it is cultured (grown, maintained) in a solution (e.g. media) including bone morphogenic protein 4 (BMP4), basic fibroblast growth factor (bFGF), and vascular endothelial growth factor (VEGF) to generate a vascular progenitor cell. In embodiments, the solution includes about 5-100 ng/ml BMP4, e.g., about 10-80, 20-50, 20-40, 10-50, 20-30, or about 25 mg/ml BMP4. In embodiments, the solution includes about 5-100 ng/ml bFGF, e.g., about 10-70, 10-50, 15-40, 15-25, or about 20 mg/ml bFGF. In embodiments, the solution includes about 5-200 ng/ml VEGF, e.g., 10-100, 20-80, 30-60, 40-60, 45-55, or about 50 ng/ml VEGF.

In embodiments, culturing the multipotent cell in step (iii) is performed in the absence of feeder cells. The multipotent cell may be allowed to grow for less than 10 days, but typically more than 2, 3, 4, 5, or 6. Thus, in embodiments, culturing the multipotent cell in step (iii) is performed for less than 10 days. In embodiments, culturing the multipotent cell in step (iii) is performed for less than 9 days. In other embodiments, the culturing the multipotent cell in step (iii) is performed for less than 8 days. In embodiments, culturing the multipotent cell in step (iii) is performed for about 7 days. In embodiments, culturing the multipotent cell in step (iii) is performed for about 8 days. In embodiments, culturing the multipotent cell in step (iii) is performed for about 9 days.

One of skill will understand that, during the culturing period, the multipotent cell or population of multipotent cells can divide and/or change character as the multipotent cell gives rise to a vascular progenitor cell. Indeed, the method is typically carried out with a plurality of multipotent cells to form a population of cells that includes, over time, increasing numbers of vascular progenitor cells. Thus, in embodiments, the method is performed with a plurality of primary cells, to form a population of cells including a plurality of vascular progenitor cells. In embodiments, the population of cells comprises 30-80% vascular progenitor cells, e.g., 35, 40, 45, 50, 60, 65, 70, 75%, or higher percentage vascular progenitor cells. In embodiments, the method further includes separating the vascular progenitor cells from the population of cells. In embodiments, the vascular progenitor cell is a CD34⁺ cell. In some cases, the vascular progenitor cells are separated from the population of cells, e.g., for further differentiation. Such separation can be carried out using any method known in the art, e.g., based on cell surface marker expression, cell size, or cell morphology.

Exemplary methods for cell separation include use of antibodies to cell surface markers (e.g., CD34, CD31, c-kit, and/or KDR for vascular progenitor cells) such as magnetic cell separation, sorting by flow cytometry, or chromatographic methods. For separation based on size, centrifugation, e.g., size exclusion centrifugation or density gradient centrifugation, can be used. See, e.g., Recktenwald (1998) Cell Separation Methods and Applications (Marcel Dekker ed.).

### B. METHODS OF GENERATING SMOOTH MUSCLE CELLS

The vascular progenitor cells formed by the methods provided herein including embodiments thereof may be used to generate smooth muscle cells and/or endothelial cells. Thus, in embodiments, the vascular progenitor cell is capable of forming a smooth muscle cell or an endothelial cell. In embodiments, the vascular progenitor cell is capable of forming a smooth muscle cell and an endothelial cell.

In embodiments, the methods further include culturing the separated vascular progenitor cells in smooth muscle cell growth media to generate smooth muscle cells. Methods for generating smooth muscle cells include culturing a vascular progenitor cell in a solution appropriate for smooth muscle cell growth (e.g., SMC growth media) and allowing the vascular progenitor cell to form a smooth muscle cell. In embodiments, the SMC growth media includes a smooth muscle cell growth factor, and the cells can optionally be grown in the presence of collagen. In embodiments, the vascular progenitor cell is cultured in SMC growth media between 6 and 20 days, e.g., at least 6, 8, 10, 12 or 14 days (i.e., the course or duration of smooth muscle cell differentiation). In embodiments, the vascular progenitor cell is cultured in SMC growth media for about 10-14 days. In embodiments, the smooth muscle cells are cultured and maintained for multiple passages, e.g., for more than 1 or 2 months. As will be understood by one of skill in the art, the cells can be passaged and the cell media changed periodically during the course of culturing.

One of skill will also appreciate that the method is typically carried out with a plurality of vascular progenitor cells, to form a population of cells that, over the course of differentiation, includes increasing numbers of smooth muscle cells. In embodiments, the percentage of smooth muscle cells in the population of cells increases to about 50-100% over the course of differentiation, e.g., more than 65, 75, 80, 85, 90, 95 or higher percentage smooth muscle cells. In embodiments, the smooth muscle cells can be further separated from the population using the methods described herein and prepared for storage (e.g., in freezing media) or therapeutic application. Smooth muscle cell surface markers that can be used for separation include alpha-SMA and calponin, though again, negative selection using an non-smooth muscle cell marker can be applied.

In another aspect, a method of generating a smooth muscle cell is provided. The method includes transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby generating the vascular progenitor cell. The vascular progenitor cell is cultured in smooth muscle cell growth media, thereby forming the smooth muscle cell. In embodiments, the vascular progenitor cell is a CD34⁺ cell.

In another aspect, an isolated smooth muscle cell generated according to the methods provided herein including embodiments thereof is provided.

### C. METHODS OF GENERATING ENDOTHELIAL CELLS

In embodiments, the methods further include culturing the separated vascular progenitor cells in endothelial cell growth media to generate endothelial cells. Methods for generating endothelial cells include culturing a vascular progenitor cell in a solution appropriate for endothelial cell growth (e.g., EC growth media) and allowing the vascular progenitor cell to form an endothelial cell. In embodiments, the EC growth media includes endothelial cell growth factor, and the cells can optionally be grown in the presence of collagen. In embodiments, the vascular progenitor cell is cultured in EC growth media between 6 and 20 days, e.g., at least 6, 8, 10, 12 or 14 days (i.e., the course or duration of endothelial cell differentiation). In embodiments, the vascular progenitor cell is cultured in EC growth media for about 10-14 days. In embodiments, the endothelial cells are cultured and maintained for multiple passages, e.g., for more than 1 or 2 months. As will be understood by one of skill in the art, the cells can be passaged and the cell media changed periodically during the course of culturing.

One of skill will also appreciate that the method is typically carried out with a plurality of vascular progenitor cells, to form a population of cells that, over the course of differentiation, includes increasing numbers of endothelial cells. In embodiments, the percentage of endothelial cells in the population of cells increases to about 50-100% over the course of differentiation, e.g., more than 65, 75, 80, 85, 90, 95 or higher percentage endothelial cells. In embodiments, the endothelial cells can be further separated from the population using the methods described herein and prepared for storage (e.g., in freezing media) or therapeutic application. Endothelial cell surface markers that can be used for separation include VE-cadherin, endoglin (CD105), vWF, and Z01 (tight junction protein), though negative selection can also be used (e.g., alpha-SMA or other non-endothelial cell marker).

In another aspect, a method of generating an endothelial cell is provided. The method includes transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby generating the vascular progenitor cell. The vascular progenitor cell is cultured in endothelial cell growth media, thereby forming the endothelial cell. In embodiments, the vascular progenitor cell is a CD34⁺ cell.

In another aspect, an isolated endothelial cell formed according to the methods provided herein including embodiments thereof is provided.

### D. CELL COMPOSITIONS

The present invention provides for methods as well as compositions to prepare vascular progenitor cells from a primary cell. Surprisingly, the vascular progenitor is formed without the requirement to reprogram a primary cell to a pluripotent stage. Upon brief exposure of a primary cell (e.g., fibroblast) with reprogramming factors (e.g., Sox2, Oct4, Klf4) for a defined period of time (e.g., 8 days) a mutlipotent cell is formed, which can subsequently be differentiated into a vascular progenitor cell. As described herein, a multipotent cell compared to a pluripotent cell, does not have the capability to go give rise to cells of all three germ layers. Therefore, a multipotent cell is a cell exhibiting lesser self-renewal capacity than a pluripotent stem cell but more self-renewal capacity than a primary cell.

In one aspect, an isolated multipotent cell is provided. The isolated multipotent cell is formed by a process including transfecting a primary cell (e.g., fibroblast) with a non-integrative reprogramming nucleic acid, thereby forming a transfected primary cell. The transfected primary cell is allowed to grow, thereby forming the multipotent cell. In embodiments, the process of forming a multipotent cell includes the steps, components, and amounts thereof, set forth above in the description of the methods of generating a vascular progenitor cell. For example, the non-integrative reprogramming nucleic acid encodes a Sox-2 polypeptide, a Oct-4 polypeptide, a Klf-4 polypeptide, a cMyc polypeptide, a Lin28 polypeptide or a p53 siRNA. In embodiments, the allowing to grow in step (ii) is performed in the absence of feeder cells. In embodiments, the allowing to grow in step (ii) is performed for less than 10 days. In embodiments, the allowing to grow in step (ii) is performed for about 8 days. In embodiments, the multipotent cell does not express a pluripotency marker. In embodiment, the pluripotency marker is a TRA1-81 polypeptide or a TRA1-60 polypeptide. In embodiments, the multipotent cell is not capable of forming an embryonic stem cell colony. In embodiments, the multipotent cell is not capable of teratoma formation.

The multipotent cell formed by the methods provided herein including embodiments thereof, may further be used to form a vascular progenitor cell by culturing the mutlipotent cell in a solution including BMP4, VEGF and bFGF. Thus, in another aspect, an isolated vascular progenitor cell is provided. The isolated vascular progenitor cell is formed by a process including transfecting a primary cell with a non-integrative reprogramming nucleic acid, thereby forming a transfected primary cell. The transfected primary cell is allowed to grow, thereby forming a multipotent cell. The multipotent cell is cultured in a solution including BMP4, bFGF, and VEGF, thereby forming the vascular progenitor cell.

### E. EXAMPLES

The following examples are offered to illustrate, but not to limit the claimed invention.

Lineage conversion of one somatic cell type into another is an attractive approach for generating specific human cell types. Lineage conversion can be direct, in the absence of proliferation and multipotent progenitor generation, or indirect, by the generation of expandable multipotent progenitor states. Here Applicants report on the development of a reprogramming methodology in which cells transition through a plastic intermediate state, induced by brief exposure to reprogramming factors, followed by differentiation. Applicants use this approach to convert human fibroblasts to mesodermal progenitor cells, including by non-integrative approaches. These progenitors cells demonstrated bi-potent differentiation potential and could generate endothelial and smooth muscle lineages. Differentiated endothelial cells exhibit neo-angiogenesis and anastomosis in vivo. This methodology for indirect lineage conversion to angioblast-like cells adds to the armamentarium of reprogramming approaches aimed at the study and treatment of ischemic pathologies.

Differentiation to Angioblast-Like Cells

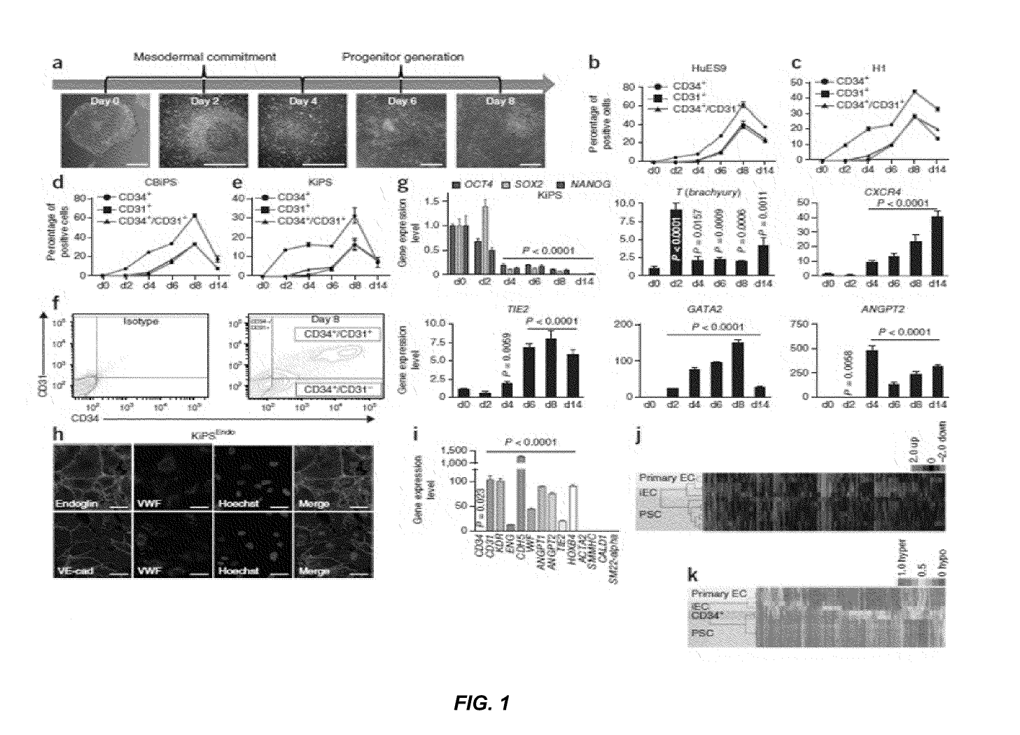

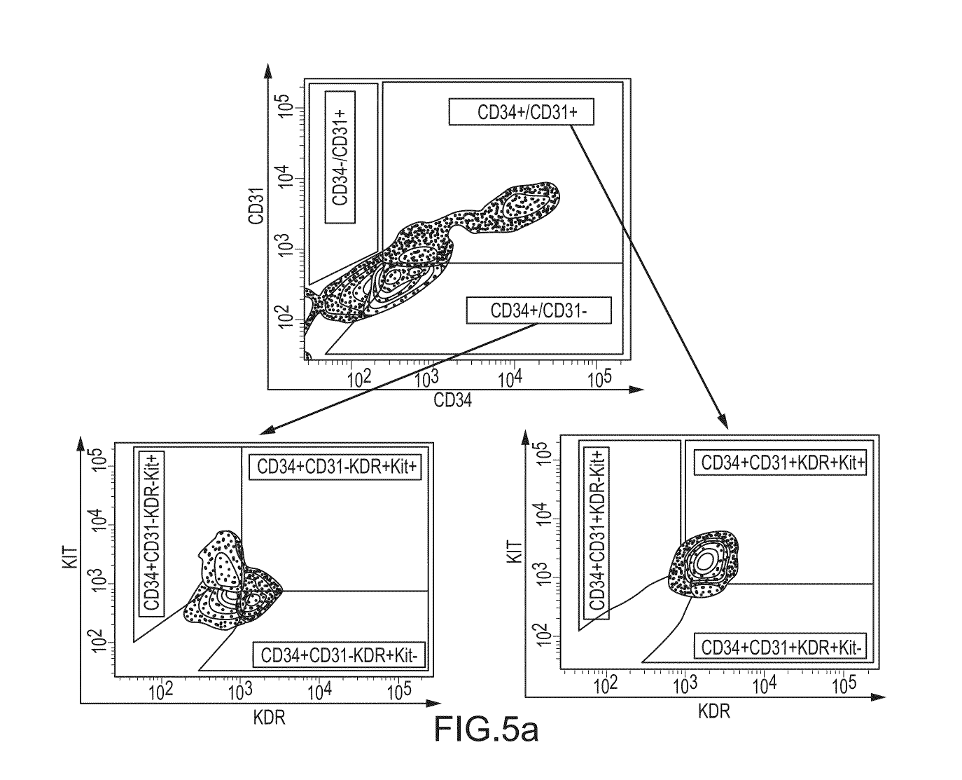

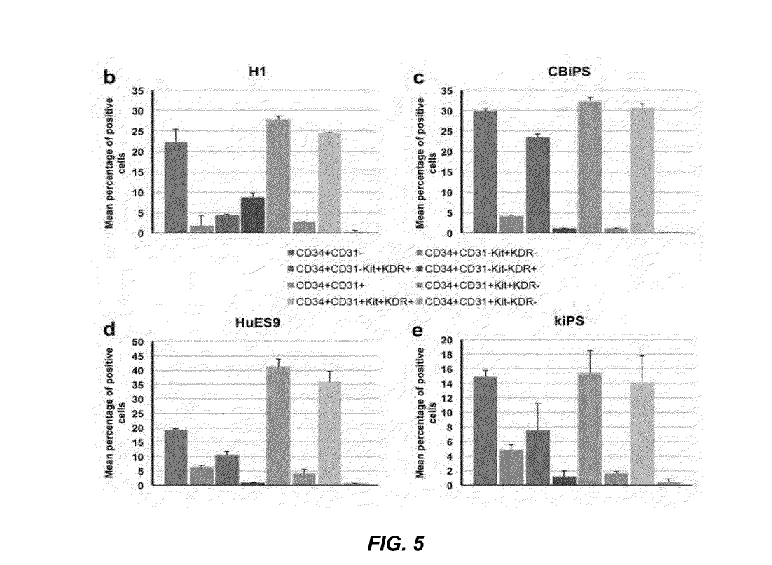

Prior to establishing Applicants' lineage conversion conditions, Applicants developed a robust media suitable for the differentiation of pluripotent stem cells (PSCs) to mesodermal progenitor cells. Applicants systematically analyzed well-known mediators of mesodermal development in different human PSC (hPSC) lines (Goldman, O., et al., Stem Cells 27, 1750-1759 (2009)). Applicants established a Mesodermal Induction Media (MIM) for efficient differentiation of hPSCs to a mesodermal fate (FIG. 1a and FIG. 5).

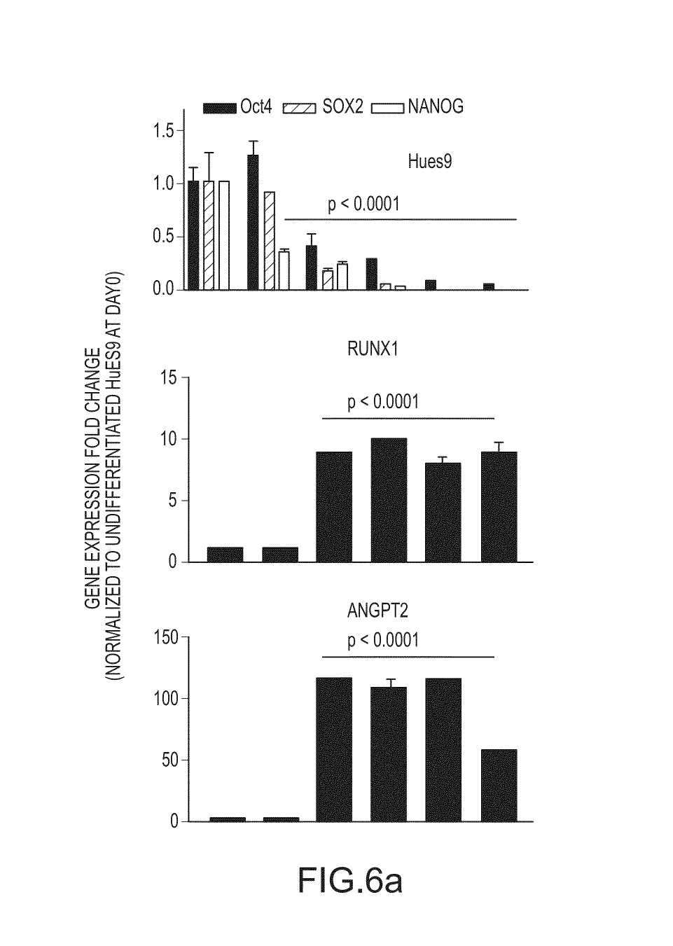

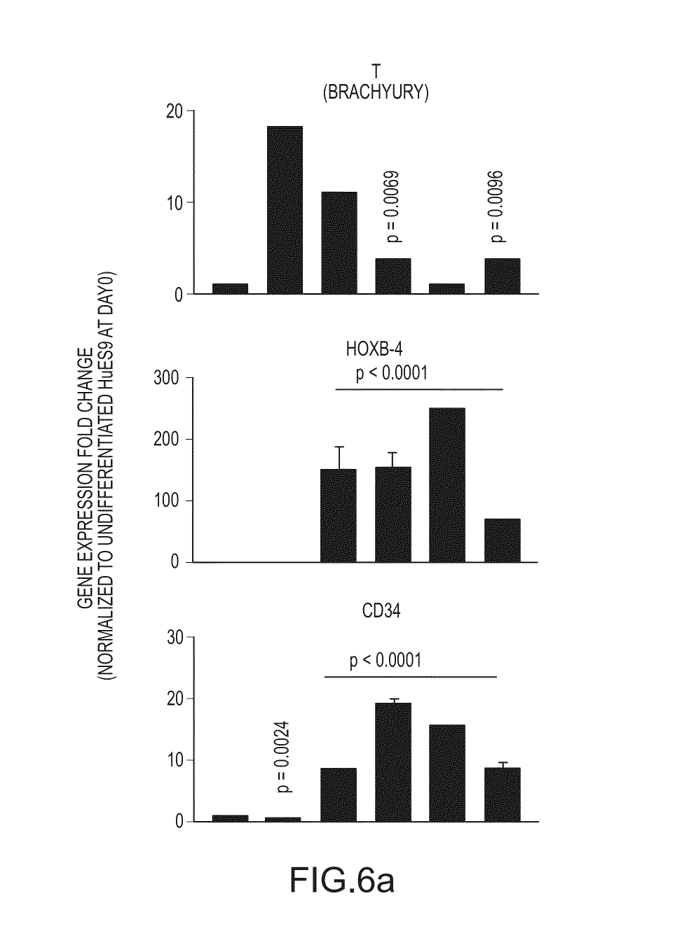

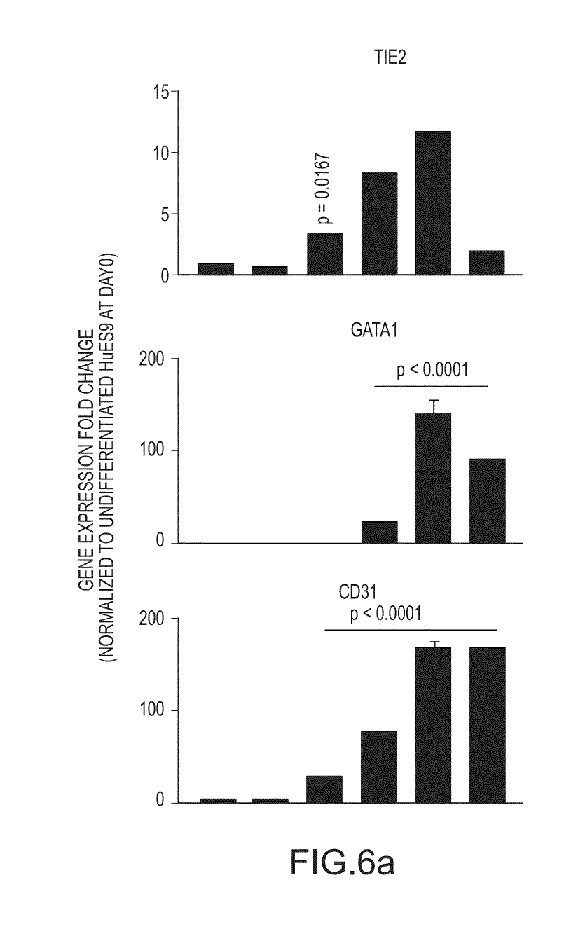

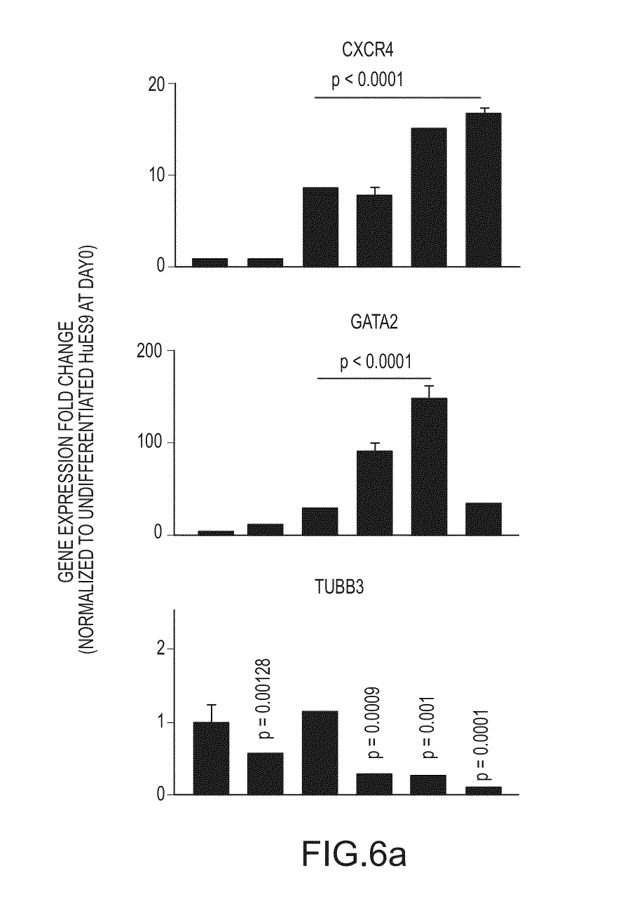

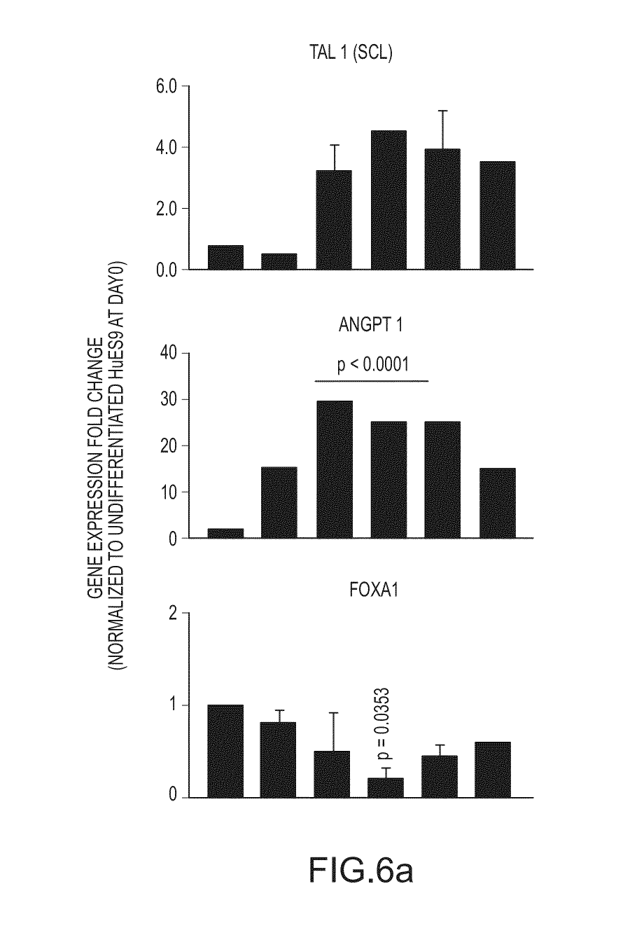

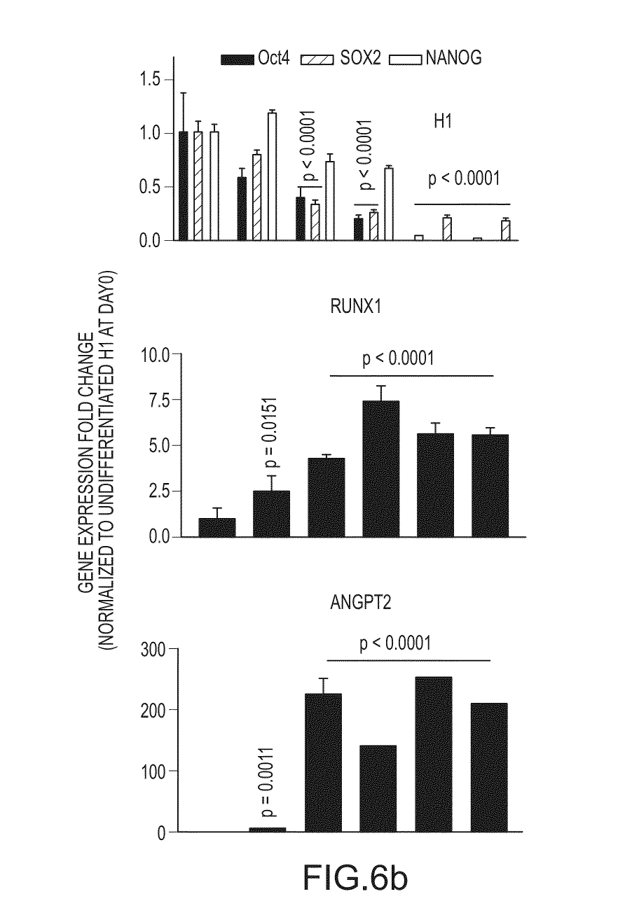

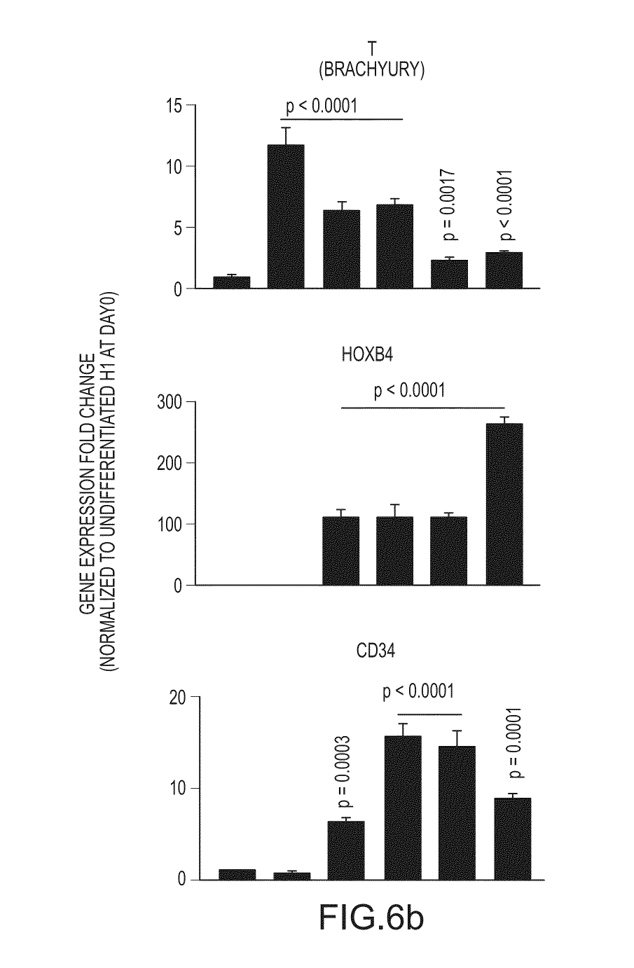

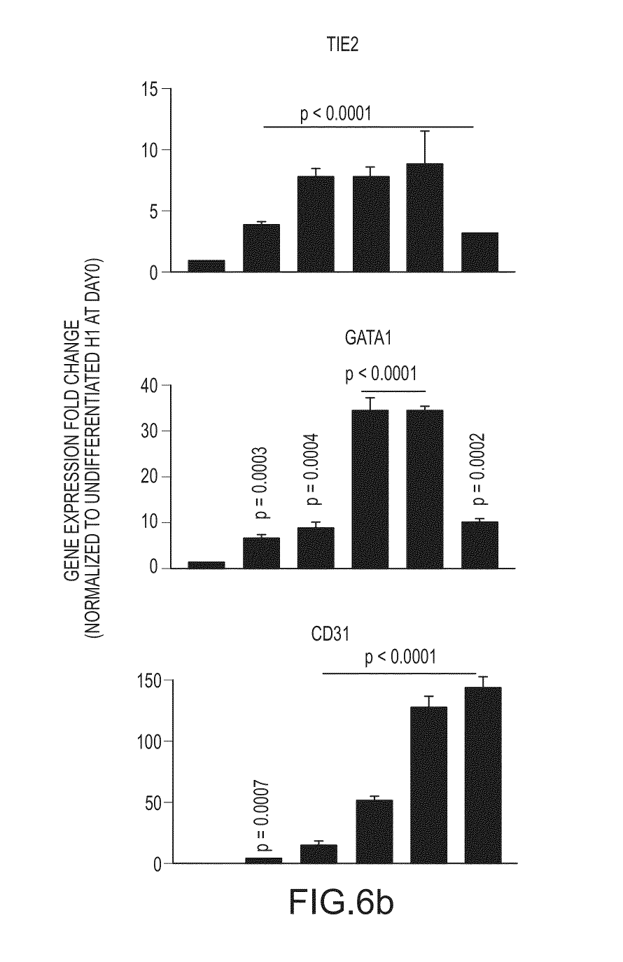

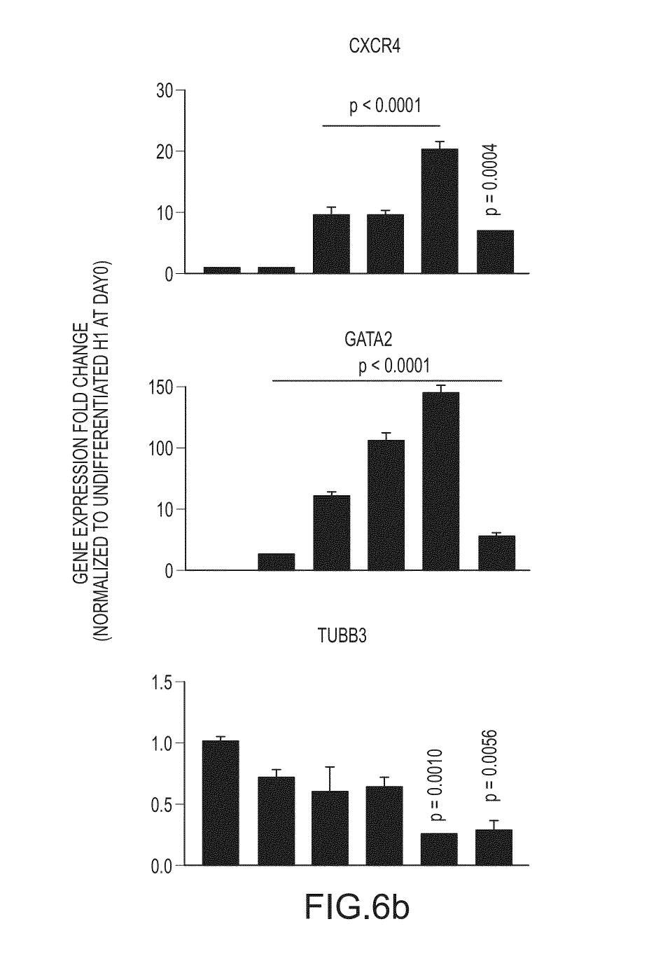

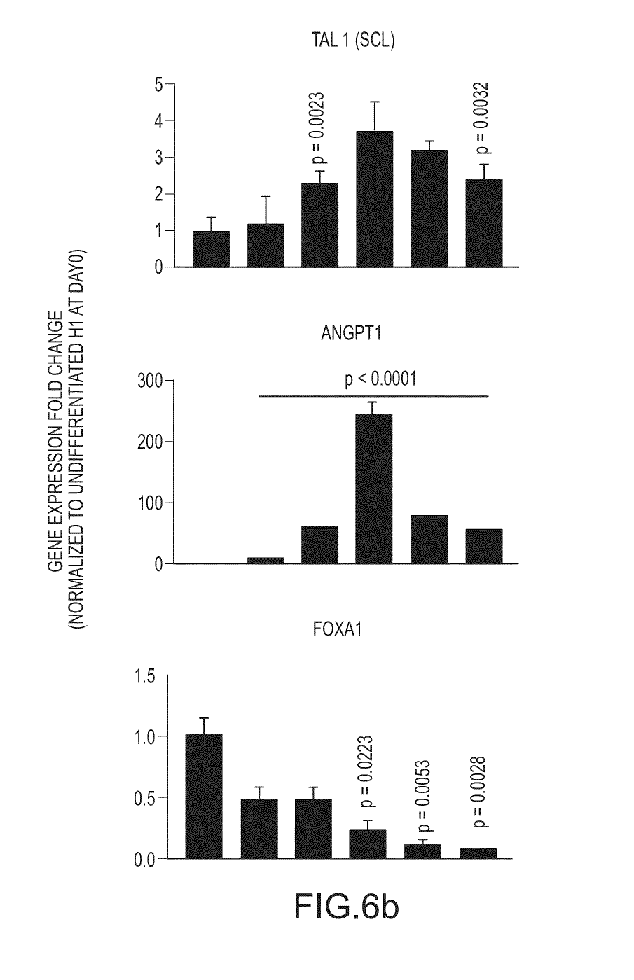

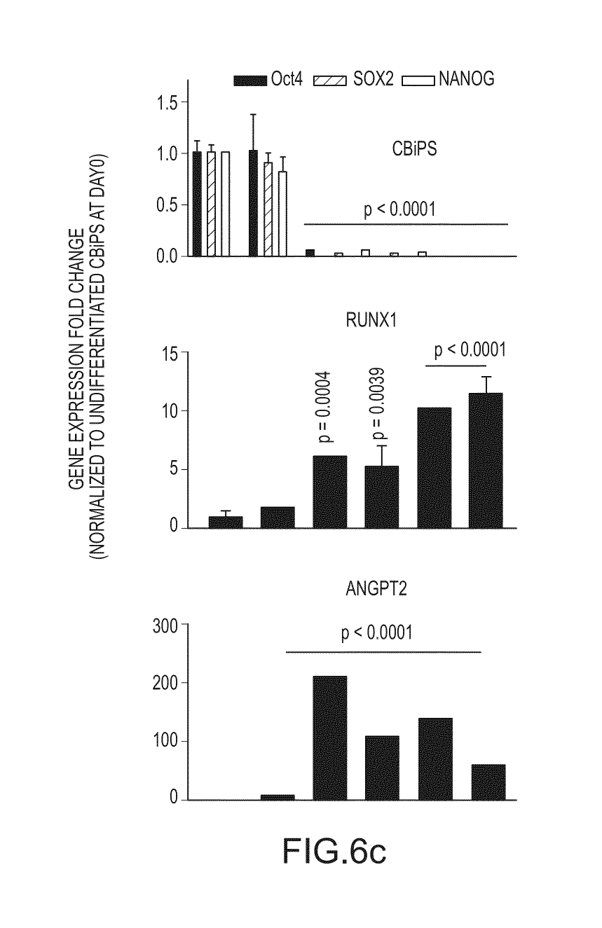

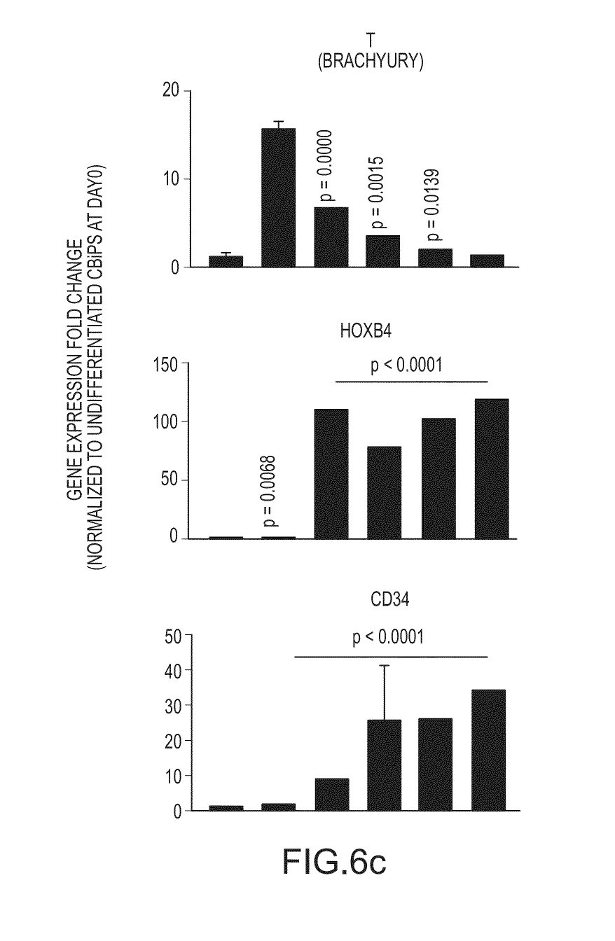

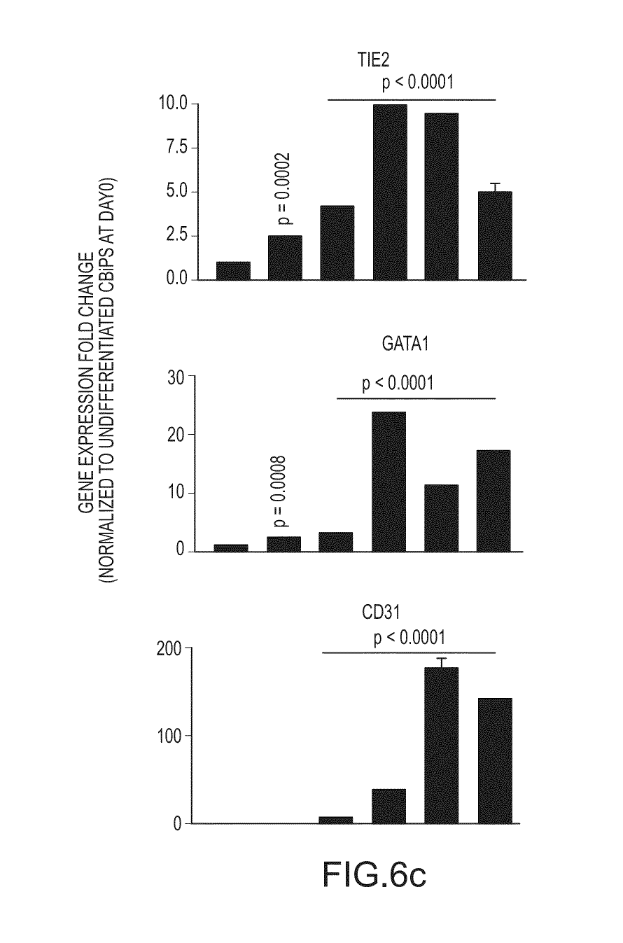

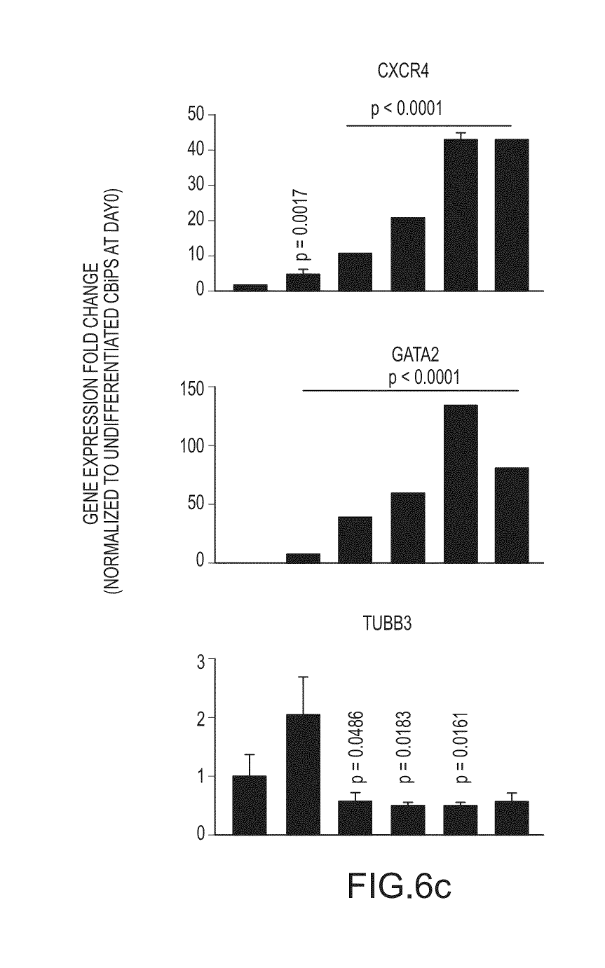

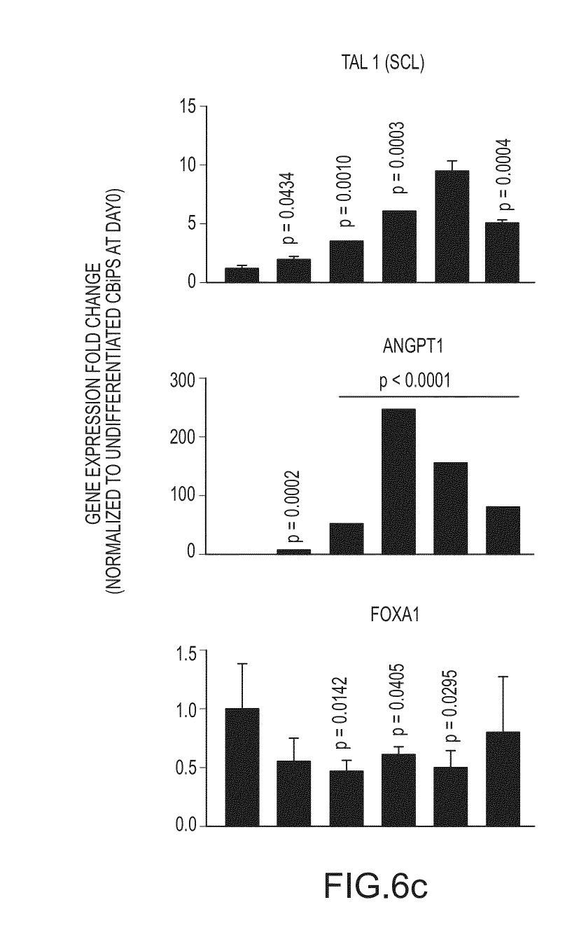

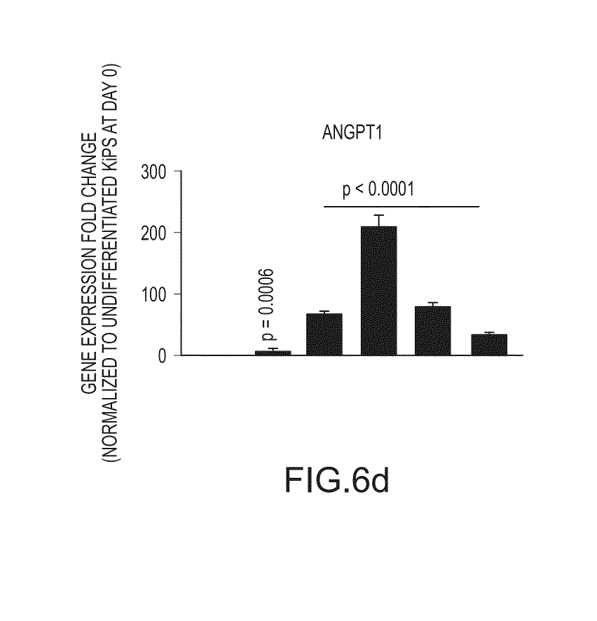

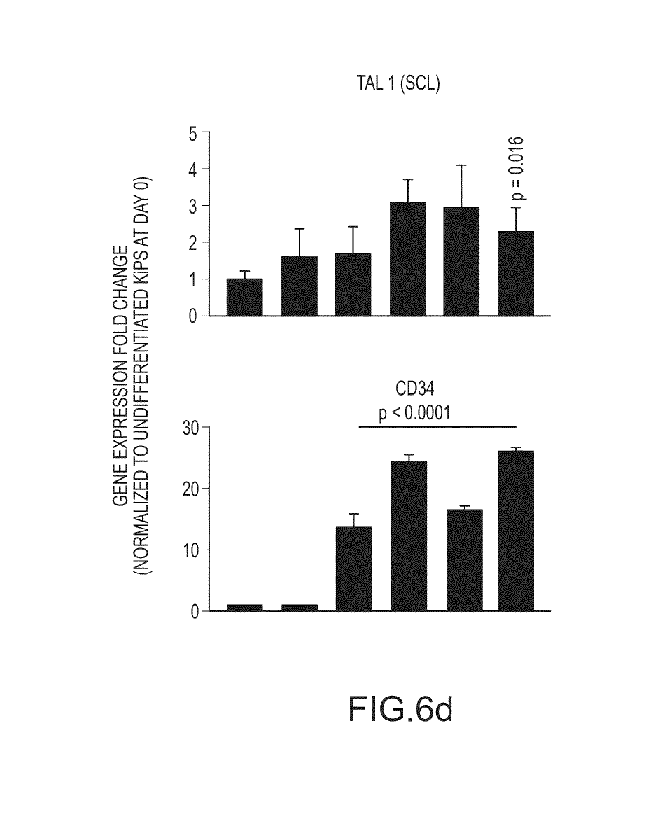

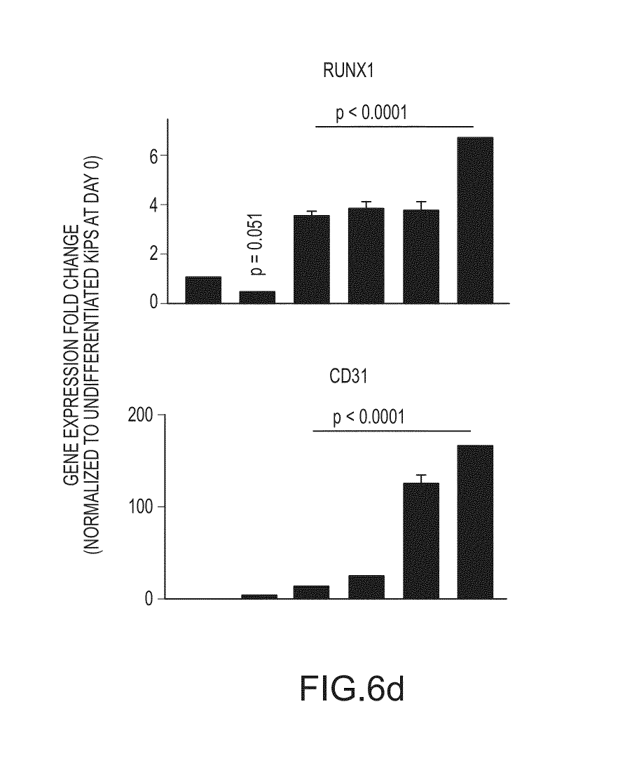

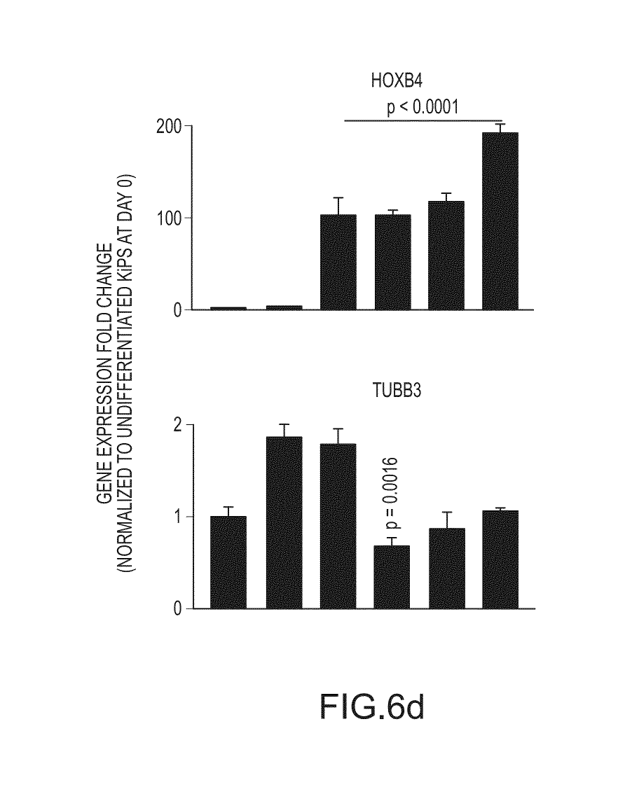

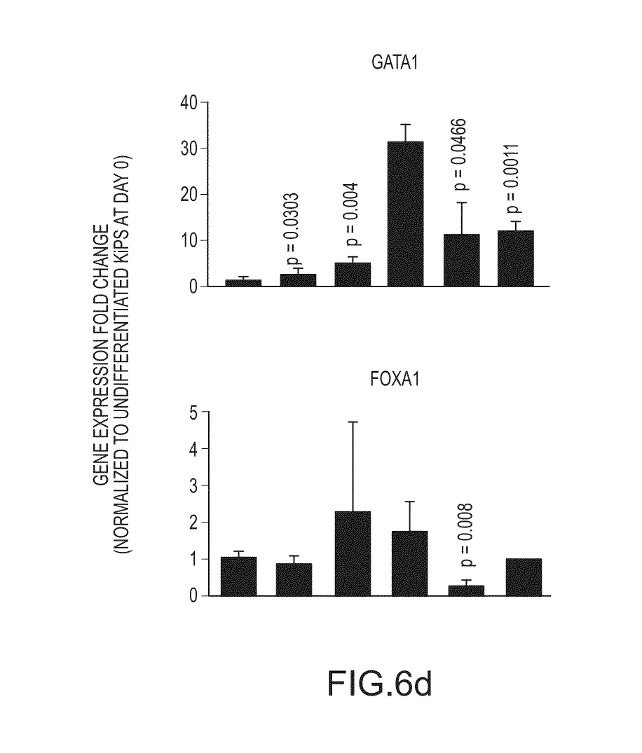

Applicants tested MIM-mediated differentiation by assessing the expression of CD34, an early marker for mesoderm-derived progenitor cells with hematopoietic and/or endothelial and smooth muscle differentiation potential, in multiple hPSC lines (FIG. 1b-f and FIG. 5-6). Applicants observed a peak of CD34+ cells by day 8 in every analyzed cell line. In parallel, Applicants observed upregulation of the vascular marker CD31 and mesodermal progenitor markers (FIG. 1b-g and FIG. 5-6), accompanied by rapid downregulation of pluripotency-related markers (FIG. 1g and FIG. 6). Additionally, Applicants observed upregulation of several early markers related to hematopoiesis, including RUNX1 and SCL/TAL1 (FIG. 6). Although this initially suggested that differentiation in MIM may lead to the generation of a tri-potent hemangioblast-like state (with hematopoietic, endothelial and smooth muscle differentiation potential), MIM-differentiated PSC-derived CD34+ cells (_{PSC}CD34+) did not result in the expression of hematopoietic markers at the protein level or the formation of hematopoietic colonies in standard assays.

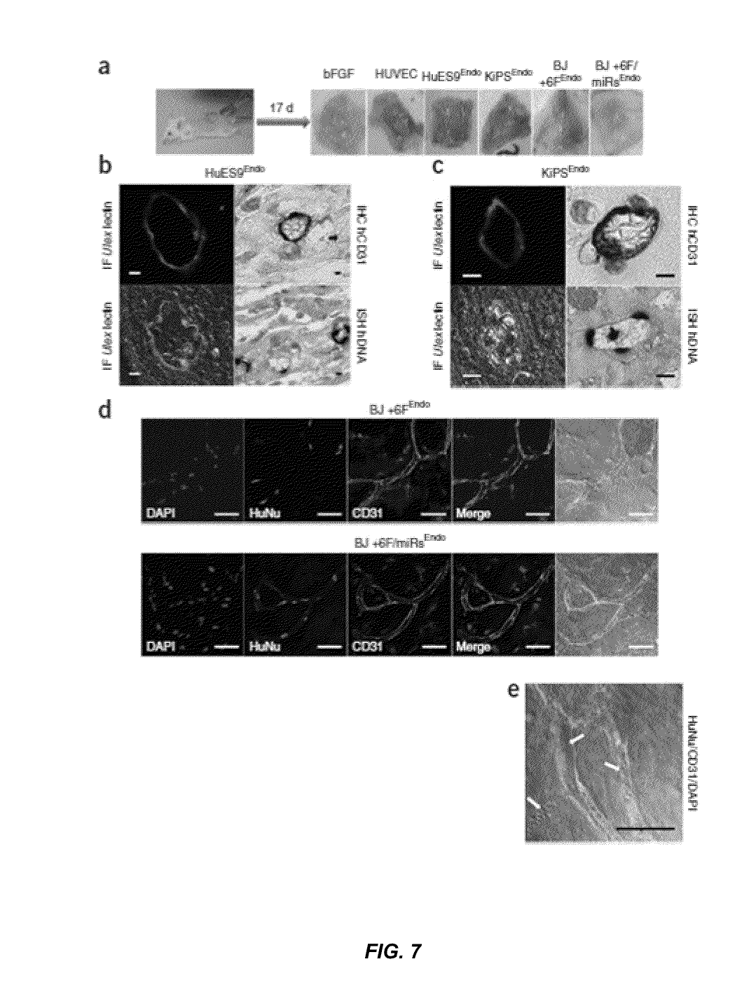

MIM-induced CD34+ cells may thus represent a developmental stage similar to that of angioblast cells (Dzierzak, E. & Speck, N. A., Nat Immunol 9, 129-136 (2008)) and Applicants consequently investigated their potential to differentiate along endothelial and smooth muscle lineages. Sorting of CD34+ cells after 8 days of MIM differentiation for mesoderm commitment, followed by subsequent differentiation towards the endothelial lineage, yielded 60-90% endoglin and VE-cadherin positive endothelial cells for all PSC lines analyzed (FIG. 1h and FIG. 7). Importantly, Applicants readily detected expression of von Willebrand factor (vWF), a mature endothelial marker and pro-coagulant protein (FIG. 1h and FIG. 7). qPCR analysis demonstrated upregulation of endothelial markers, but not of smooth muscle markers (FIG. 1i and FIG. 7).

Similarly, Applicants sorted _{PSC}CD34+ cells and subjected them to smooth muscle differentiation conditions, yielding more than 50% of smooth muscle cells as indicated by immunofluorescence staining (FIG. 8). qPCR demonstrated significant upregulation of smooth muscle markers, including expression of high molecular weight Caldesmon type 1 (CALD1), but not of endothelial markers (FIG. 8). Single cell differentiation assays demonstrated that the MIM-differentiated _{PSC}CD34+ cells are multipotent (FIG. 8).

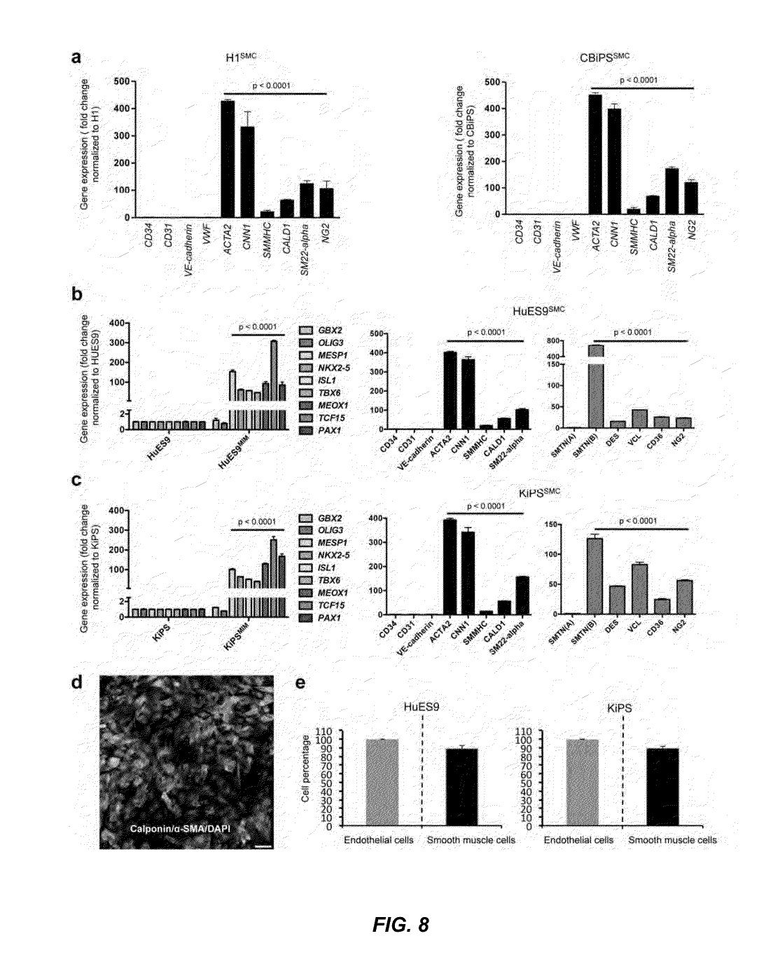

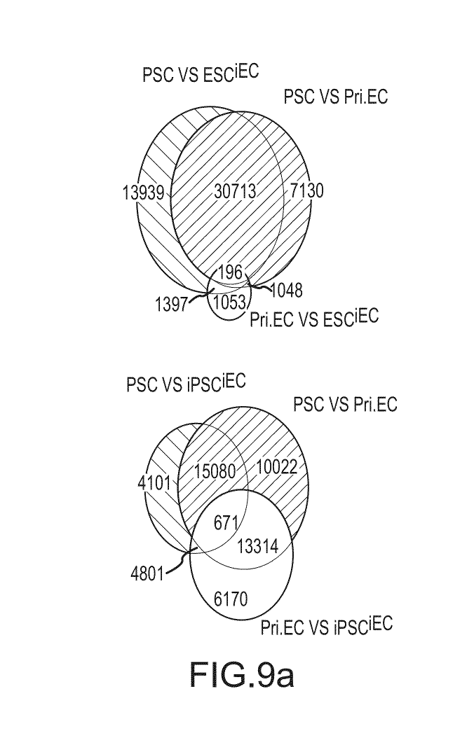

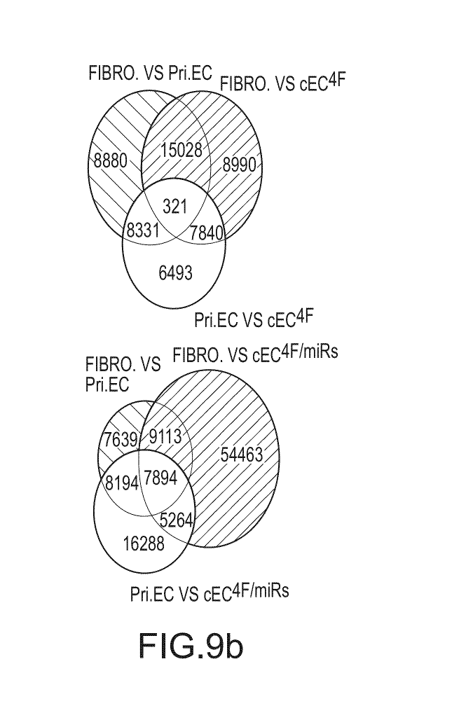

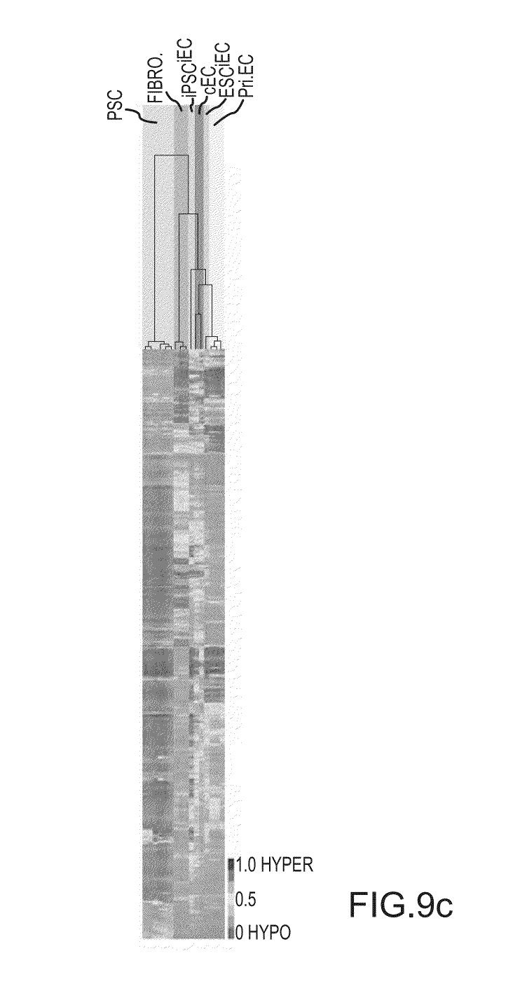

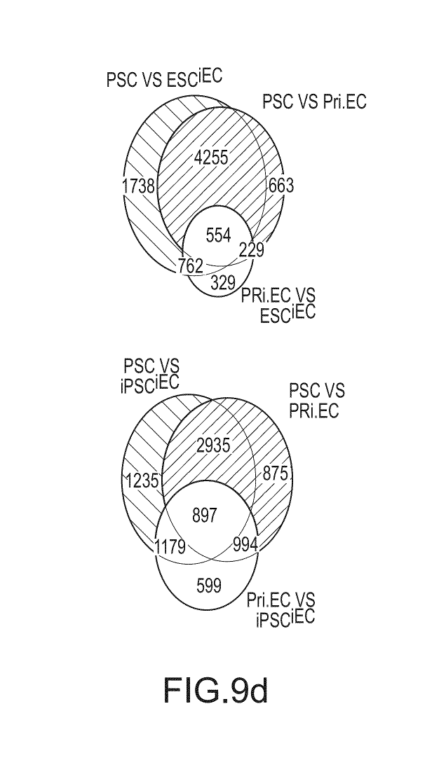

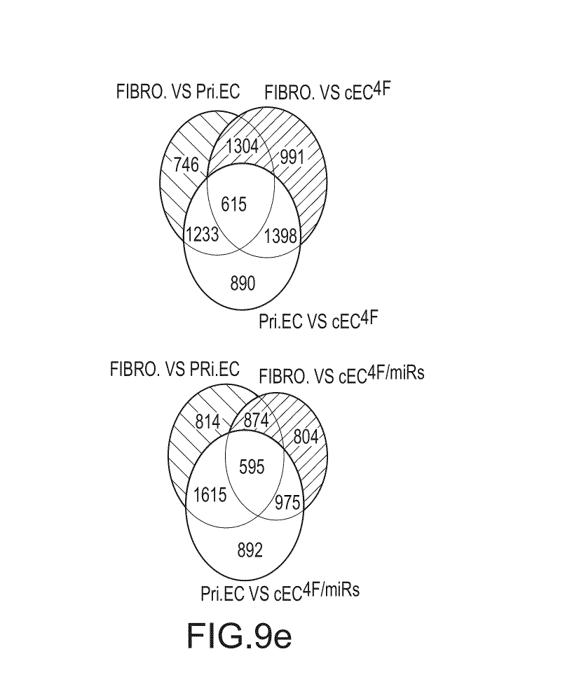

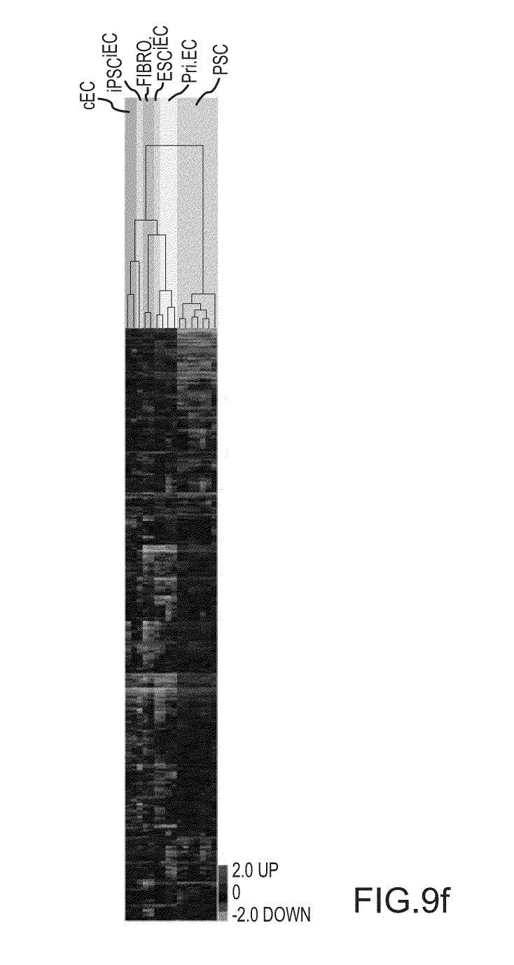

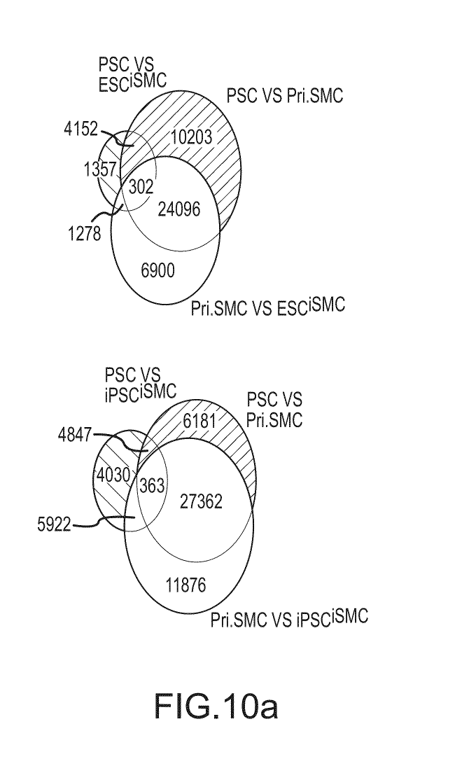

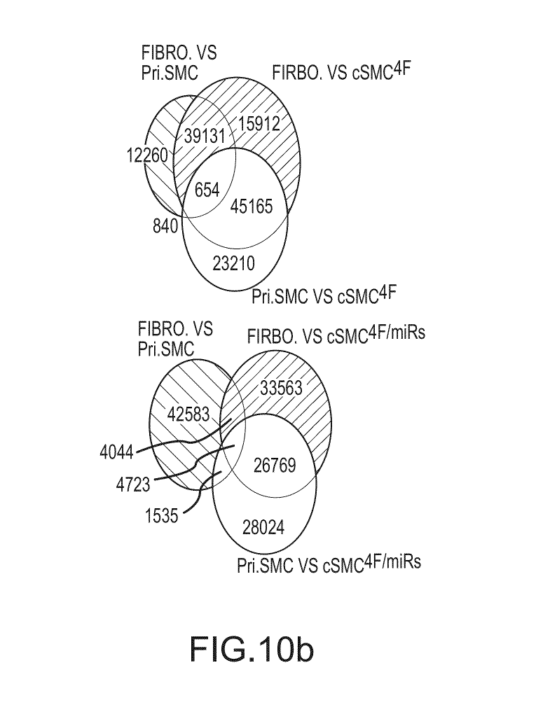

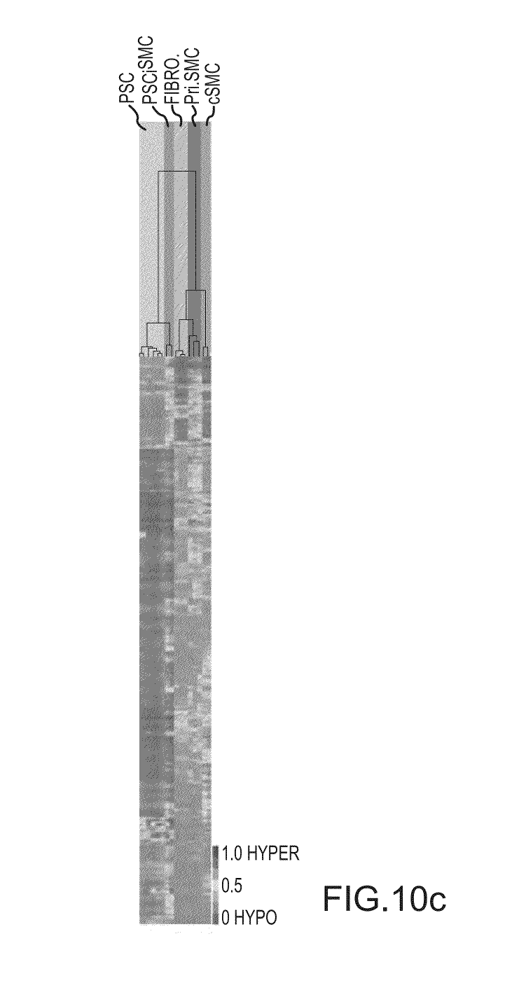

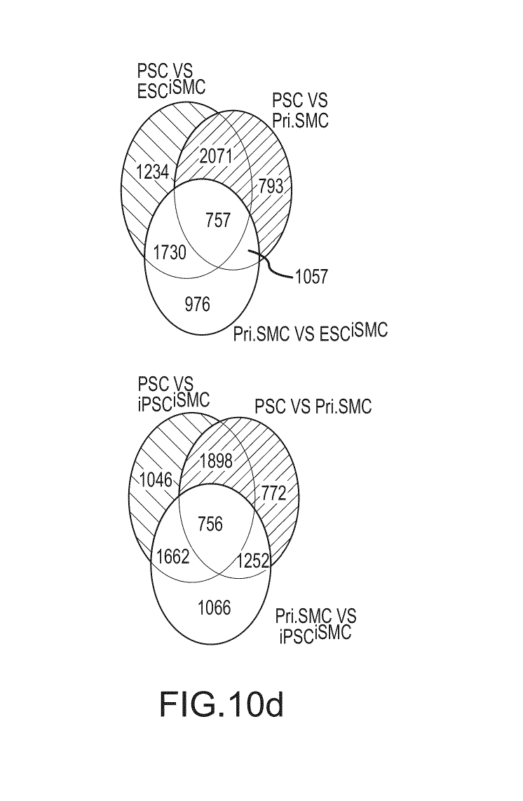

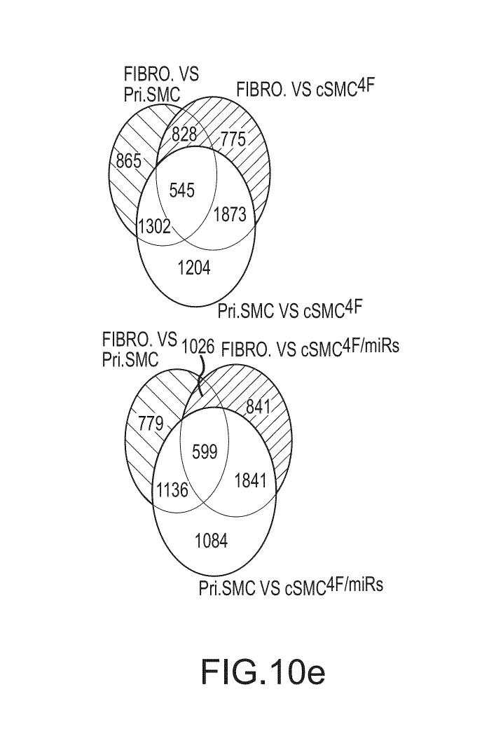

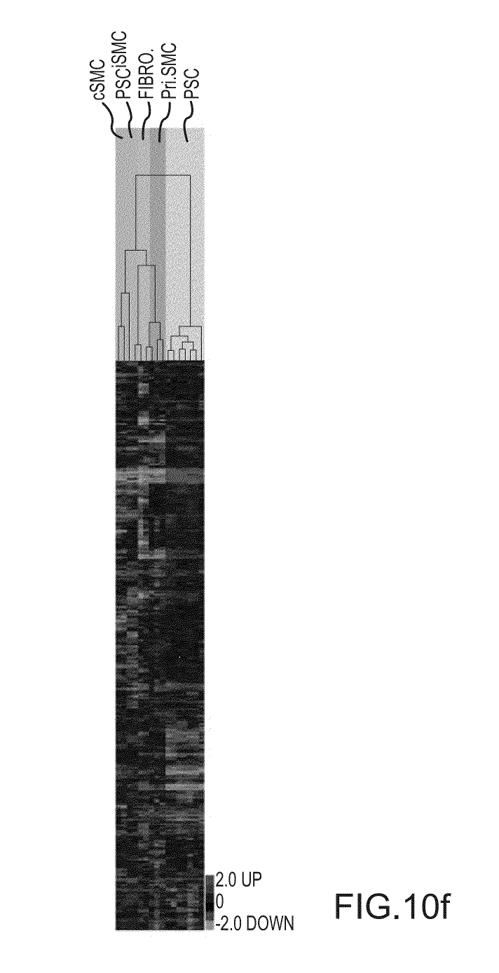

Applicants' data thus indicate that MIM can be used for differentiation of hPSCs to CD34+ cells with the potential to generate both endothelial and smooth muscle lineages. Moreover, CD34+ cells are generated more efficiently, depending on the PSC line but at least 30%, than in previously described protocols (Choi, K. D., et al., Stem Cells 27, 559-567 (2009); James, D., et al., Nat Biotechnol 28, 161-166 (2010); Levenberg, S. et al., Nat Protoc 5, 1115-1126 (2010)). Genome-wide DNA methylation and gene expression studies indicated a clear distinction between all differentiated cells and undifferentiated PSCs at both the transcriptome and the methylome level (FIGS. 1j,k and FIGS. 9, 10) although Applicants observed some differences between PSC-endothelial differentiated cells and primary endothelial cells (see Discussion).

Conversion of Human Fibroblasts to Angioblast-Like Cells

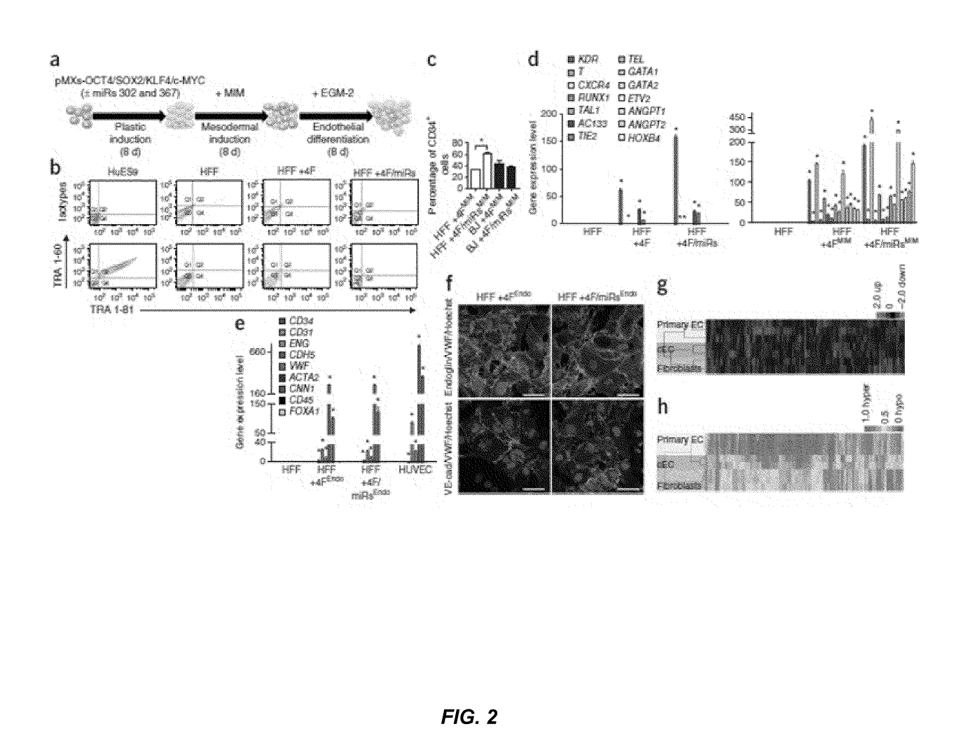

Applicants next asked whether MIM could be coupled to partial de-differentiation or ‘plastic’ induction to convert human fibroblasts into CD34+ angioblast-like progenitor cells (_{Fib}CD34+). Applicants induced plasticity by by short-term exposure of fibroblasts to iPSC reprogramming conditions (Efe, J. A., et al., Nat Cell Biol 13, 215-222 (2011); Kim, J., et al., Proc Natl Acad Sci USA, 108, 7838-7843 (2011)), followed by MIM differentiation (FIG. 2a). Applicants first used retroviral approaches and the traditional four-factor combination (SOX2, Oct4, KLF4 and c-Myc) in two neonatal human fibroblast lines (HFF and BJ) and in adult human dermal fibroblasts (HDF). An 8-day exposure of cells to reprogramming factors and iPSC-like culture conditions, followed by MIM differentiation for an additional 8-day period, led to the appearance of a prominent _{Fib}CD34+ population (FIGS. 2b,c). Of note, precise frequency analysis was technically difficult because Applicants' procedure involves non-clonal expanding populations of cells. Applicants calculated “angioblast conversion efficiencies” by estimating the ratio between the final number of converted cells and the initial number of fibroblasts. Taking into account that 75,000 fibroblasts give rise to ˜2×10⁶ cells by the end of MIM commitment, of which 20-60% are CD34+ (depending on the cell line of origin and the method used for plasticity induction), conversion efficiencies range between 400-1200%.

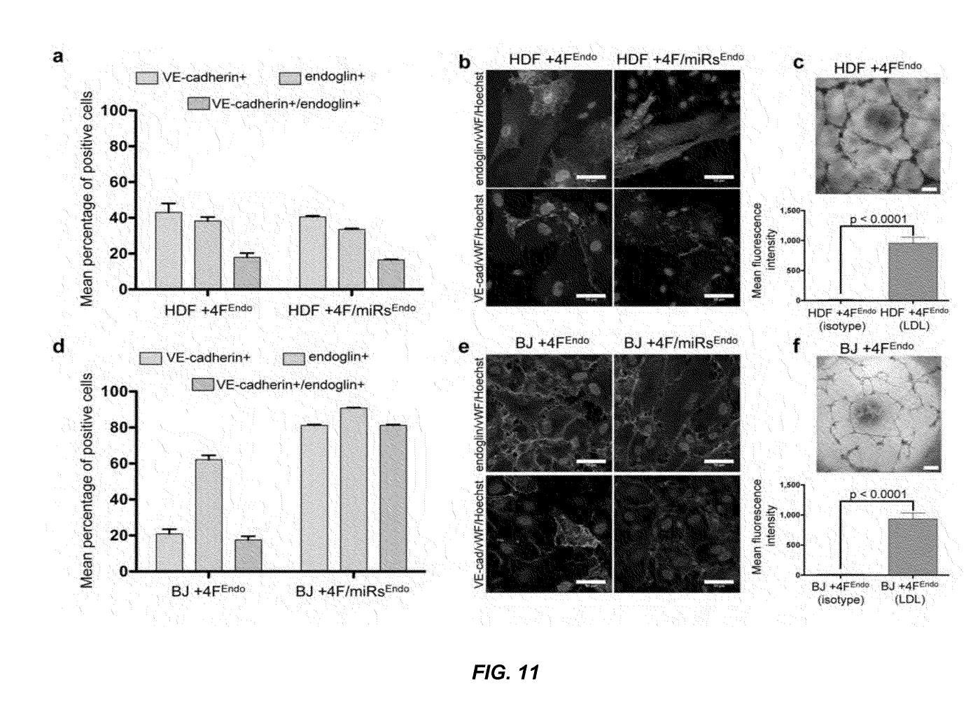

Applicants additionally asked whether the miR 302-367 clusters, demonstrated to play a role during reprogramming towards iPSCs (Anokye-Danso, F., et al., Cell Stem Cell 8, 376-388 (2011); Subramanyam, D., et al., Nat Biotechnol 29, 443-448 (2011)), could increase the efficiency of the process. Applicants observed that inclusion of miR 302-367 improved the efficiency of _{Fib}CD34+ cell generation in some but not all lines (FIG. 2c and FIG. 11). Applicants next sought to determine the minimal requirements for the conversion of human fibroblasts into _{Fib}CD34+ by systematic single factor removal. Applicants observed marginal levels of CD34+ cells, with low fluorescence intensities, when SOX2 was employed alone (˜5% CD34^{low}), and subsequent differentiation of sorted CD34+ cells did not yield endothelial or smooth muscle cells. Altogether, combination of the four Yamanaka factors, alongside the use of iPSC-like culture conditions, was necessary for the conversion, into _{Fib}CD34+ with bi-potent differentiation potential resembling that of an angioblast-like state.

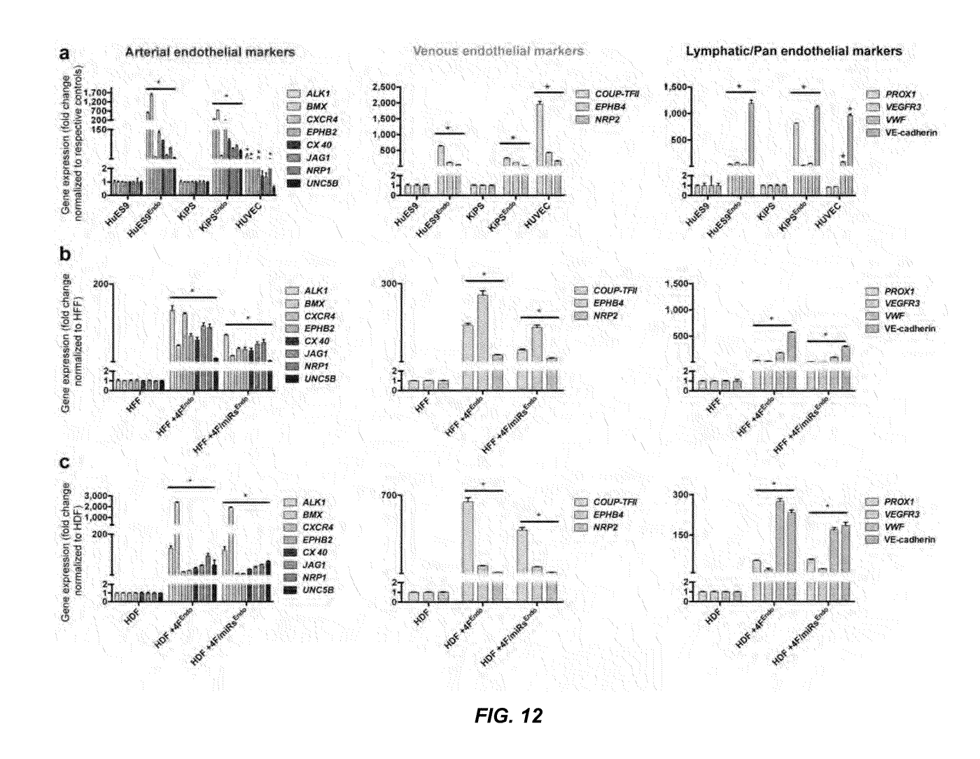

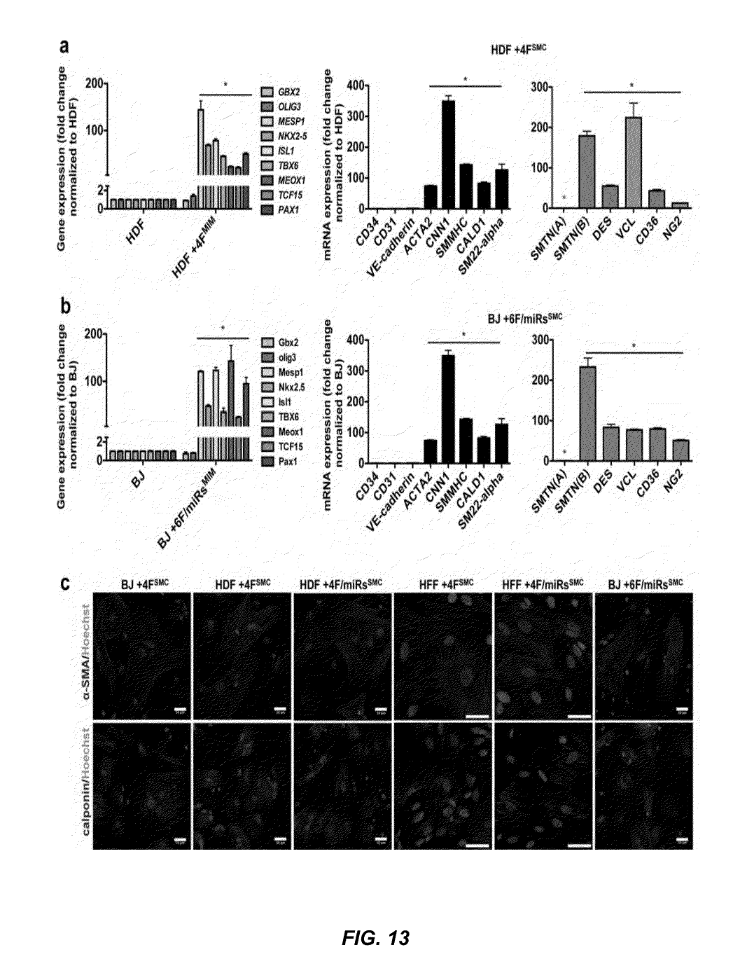

Similarly to PSC differentiation, MIM differentiation led to a significant upregulation of angioblast-related markers in all conditions analyzed (FIG. 2d). Sorting of MIM-differentiated _{Fib}CD34+ cells and subsequent culture in media promoting endothelial or smooth muscle cell differentiation resulted in the upregulation of lineage-specific markers at both the RNA and protein levels (FIGS. 2e,f and FIG. 11). Lineage conversion of human fibroblasts towards the endothelial lineage resulted in the mixed expression of different endothelial sub-types markers including expression of arterial, venous and lymphatic endothelial genes (Narazaki, G., et al., Circulation 118, 498-506 (2008)) (FIG. 12). Similarly, analysis of smooth muscle cell populations derived from human fibroblasts demonstrated mixed expression of smooth muscle markers (Cheung, C. et al., Nat Biotechnol 30, 165-173 (2012)) including expression of the pericyte marker NG2 (FIG. 13).

Converted endothelial cells lost many features of fibroblast gene expression and DNA methylation profiles and acquired characteristics of primary endothelial cells (FIGS. 2g, h and FIG. 9). When all samples were compared by unsupervised hierarchical clustering of the mRNA and methylation array data, regardless of their method of derivation, two clear major groups were observed, pluripotent cells and differentiated cells. As expected fibroblasts clustered more closely to differentiated cells than to PSCs (FIGS. 9 and 10). Both mRNA expression and DNA methylation results were very similar in terms of describing the relationships among the different cell types (FIGS. 9 and 10).

Altogether, Applicants' results demonstrate that 8-day exposure of human fibroblasts to iPSC reprogramming factors and iPSC culture conditions induced an intermediate plastic state. Subsequent mesodermal induction by 8-day exposure to MIM yielded intermediate CD34+ bi-potent progenitor populations, which could be further differentiated to endothelial and smooth muscle cell populations.

Conversion to Angioblasts by Non-Integrative Approaches

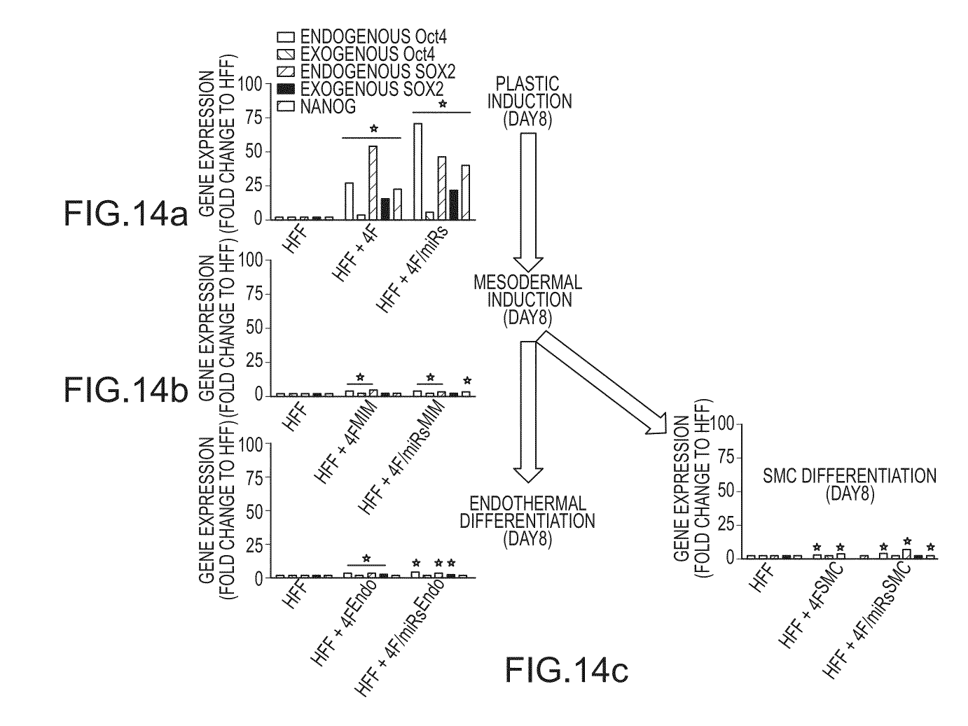

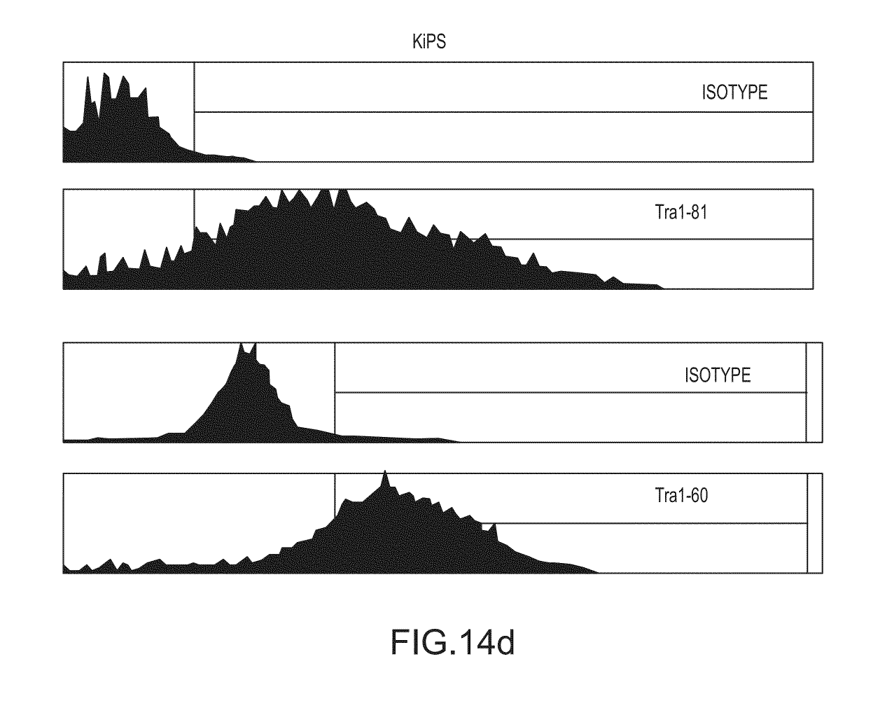

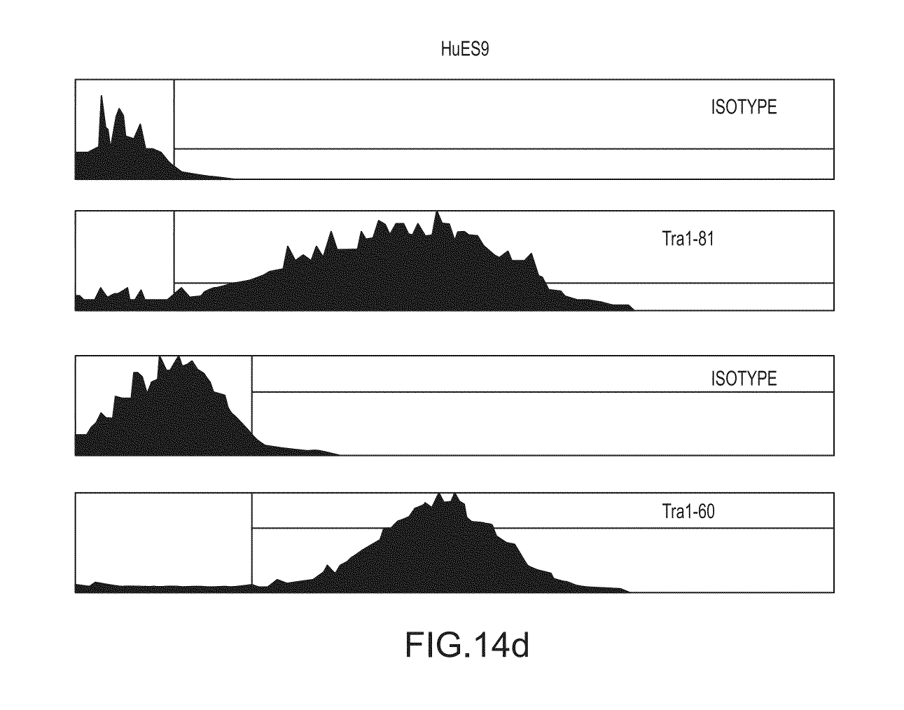

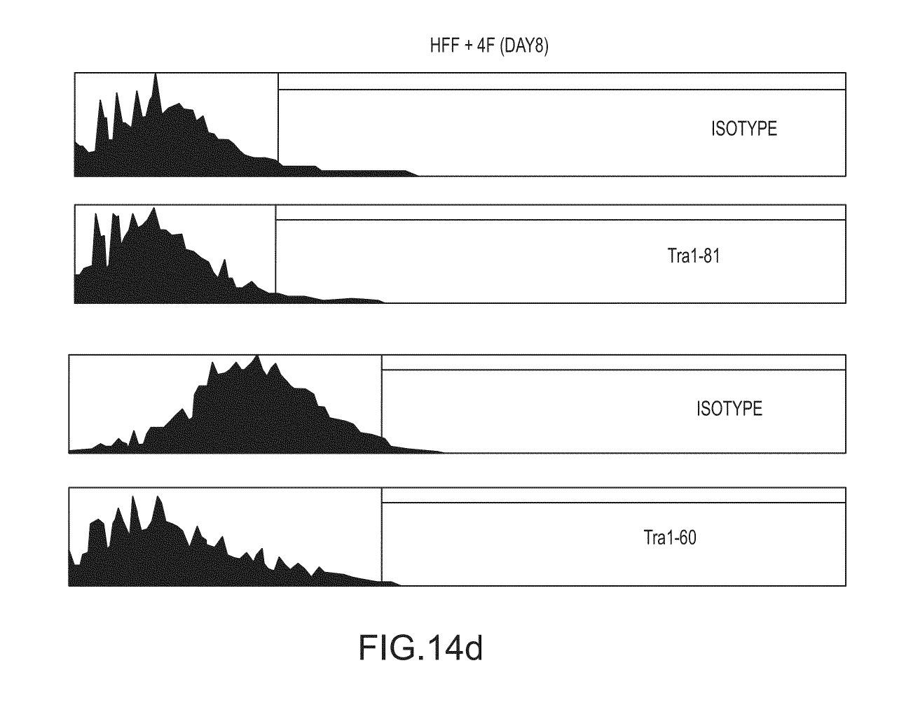

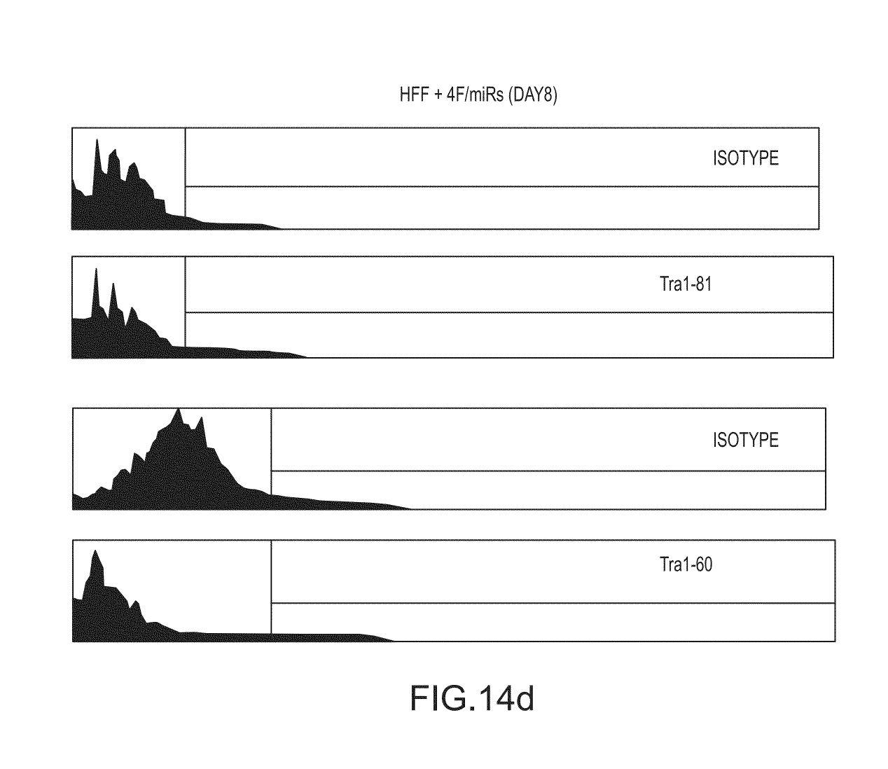

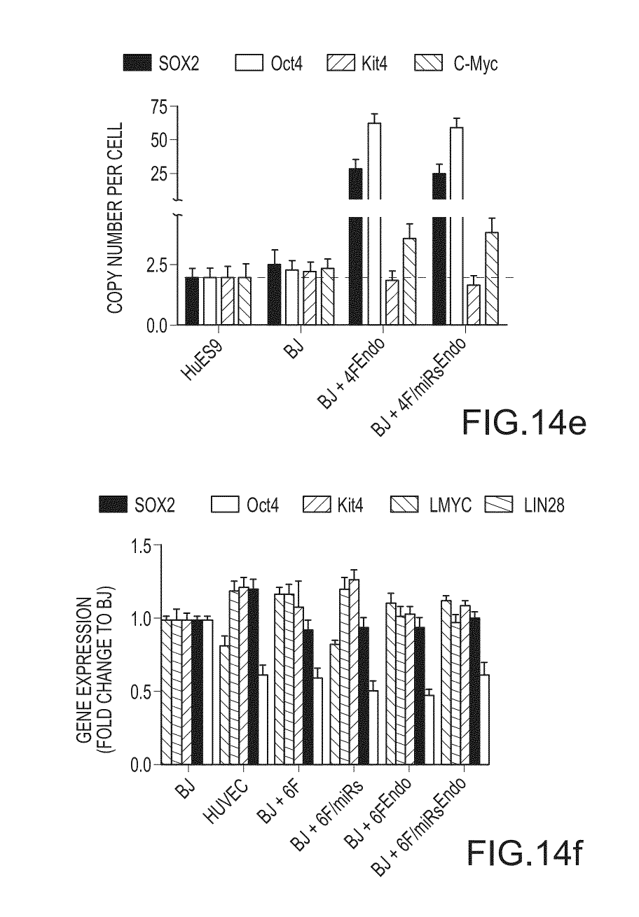

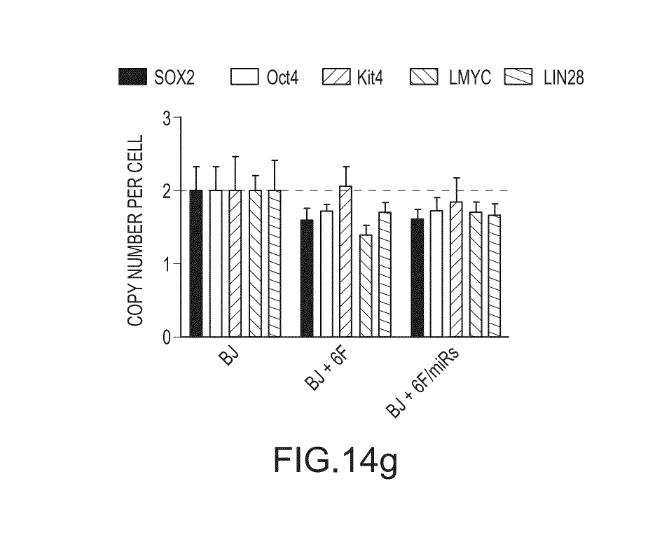

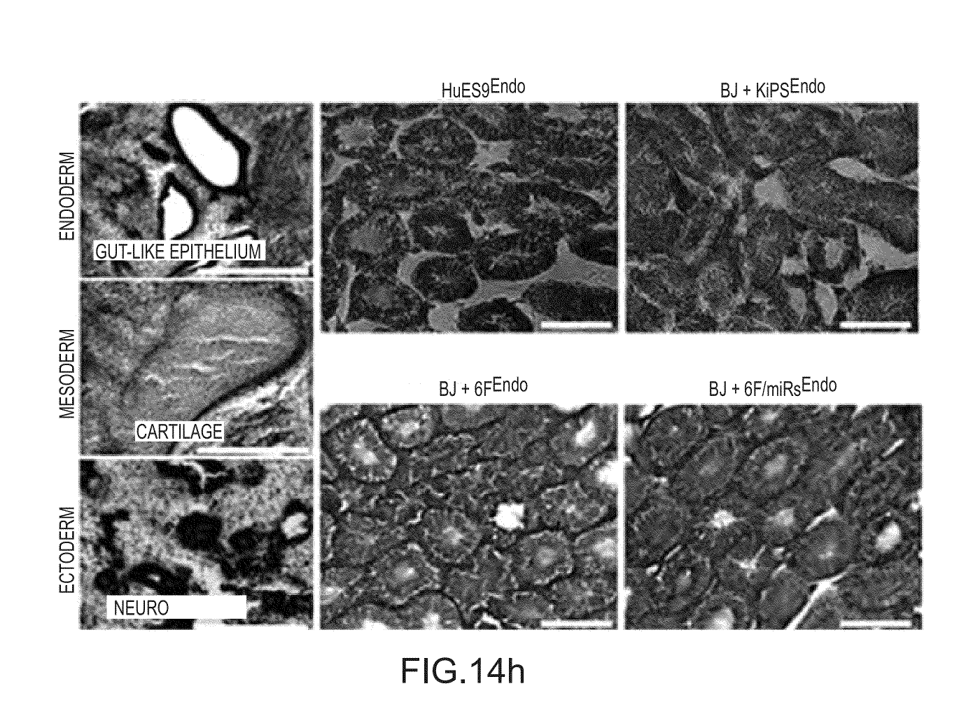

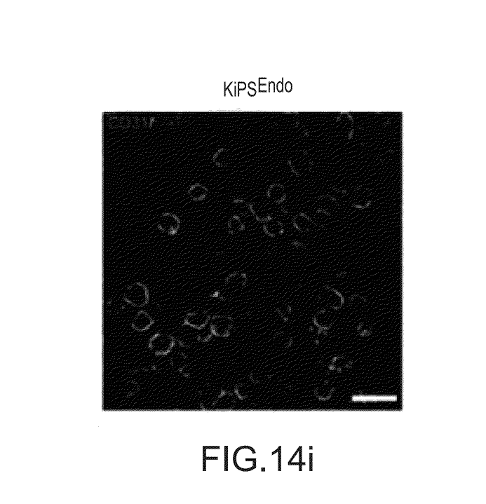

Applicants next investigated whether lineage conversion could result in intermediate pluripotent cell population even in non-permissive iPSC reprogramming conditions. TRA1-81 and TRA1-60 are markers for pluripotent cells, the latter recently described as the most reliable early marker for iPSC generation, with a success prediction rate of up to 90% (Chan, E. M., et al. Nat Biotechnol 27, 1033-1037 (2009)). Applicants' retroviral approach for plastic induction of fibroblasts did not lead to the expression of either TRA1-60 or TRA1-81 (FIG. 2b and FIG. 14). Importantly, Applicants observed residual expression of the Yamanaka factors transgenes upon differentiation to CD34+ cells as well as their endothelial and smooth muscle derivatives (FIG. 14). Applicants thus pursued the establishment of non-integrative approaches for conversion of human fibroblasts to xxx.

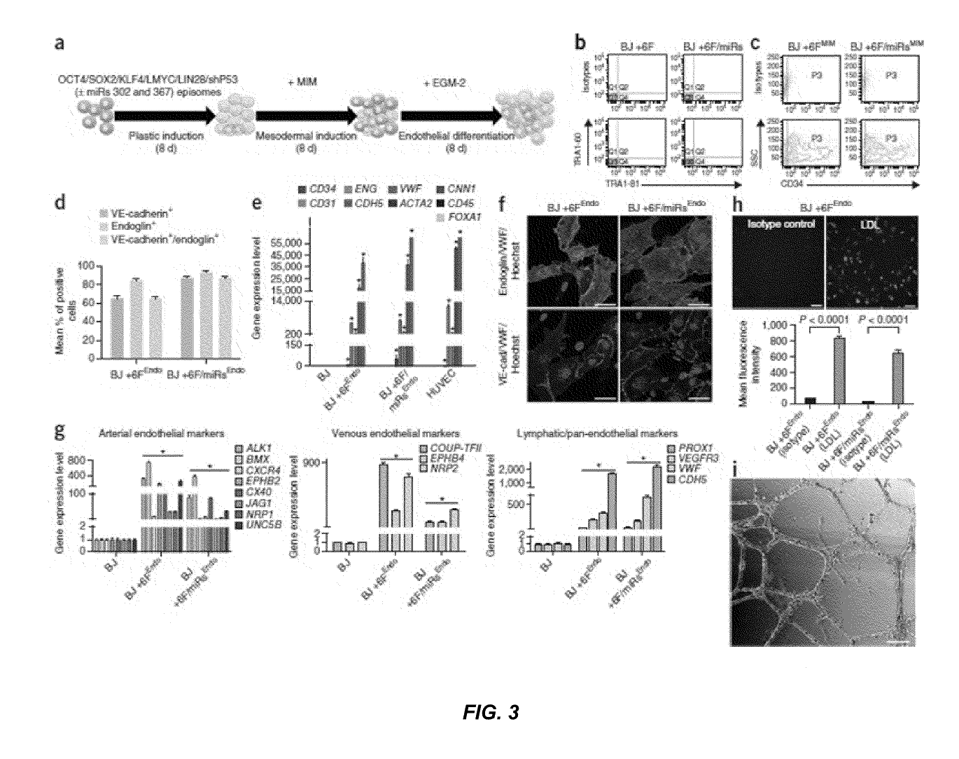

Applicants chose a six-factor combination (Oct4, SOX2, KLF4, non-transforming LMYC (MYCL1), LIN28 and shRNA against p53) proven to generate human iPSCs in the presence of murine feeder layers when delivered episomally (Okita, K., et al., Nat Methods 8, 409-412 (2011)) (FIG. 3). Applicants obtained plastic reprogramming intermediates by electroporation of each vector followed by a 6-day resting phase prior to switching to iPSC-like reprogramming conditions in the presence of WiCell media (FIG. 3a). After 8 days, the media was changed to MIM for an additional 8 days yielding CD34+ cells. Expression of TRA1-60 and TRA1-81 remained undetectable (FIGS. 3b,c). Sorting of _{Fib}CD34+ cells and subsequent differentiation into endothelial and smooth muscle lineages resulted in the upregulation of cell-type specific markers at both the RNA and protein level (FIG. 3d-g). As observed previously, the generated endothelial and smooth muscle cells represented mixed populations of different sub-types (FIG. 3g).

Applicants observed the rapid clearing of episomal vectors and did not detect random integration of exogenous genes in the differentiated endothelial cells (FIG. 14). Furthermore, testis injection of one million differentiated endothelial cells did not result in teratoma formation in any of the groups analyzed after 10 weeks, including cells generated by differentiation of PSCs (FIG. 14).

Converted Cells are Functional In Vitro and In Vivo

Two well-characterized physiological hallmarks of smooth muscle cell function are their calcium responses and their contractility (Cheung, C. et al., Nat Biotechnol 30, 165-173 (2012); Ohta, T., Ito, S. & Nakazato, Y., Br J Pharmacol 112, 972-976 (1994); Kohda, M. et al., J Physiol 492 (Pt 2), 315-328 (1996)). Contraction of _{Fib}CD34+ cell-derived smooth muscle cells occurred both spontaneously as well as upon drug stimulation (FIG. 15). Exposure to carbachol resulted in rapid calcium transients. Of note, HEK293T cells also show calcium transients in response to carbachol, but they do not physically contract as demonstrated by their unchanged cell surface area (FIG. 15).

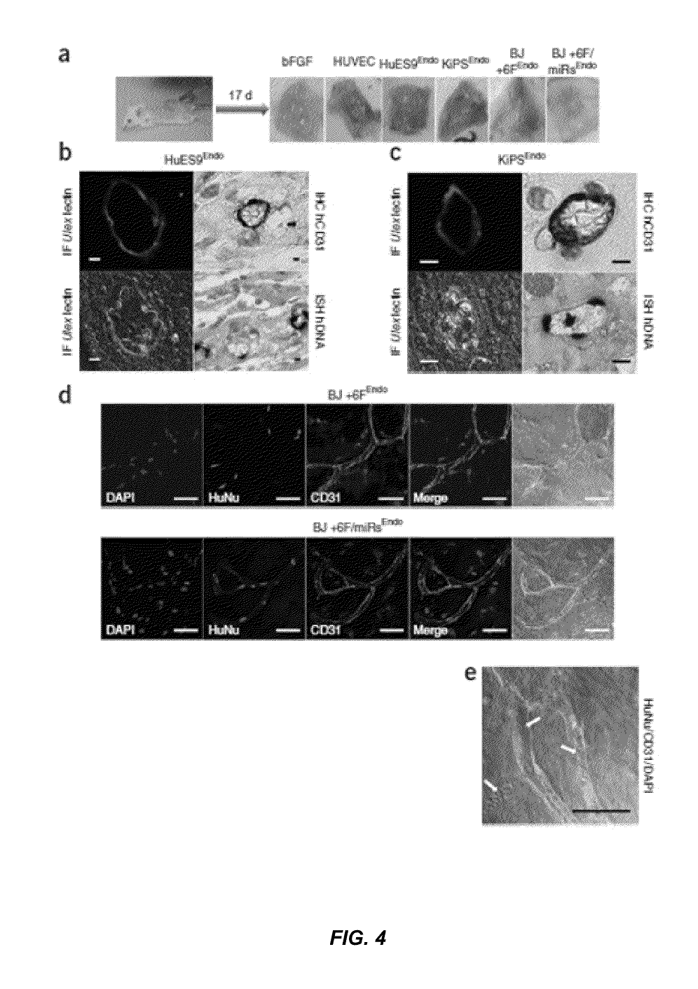

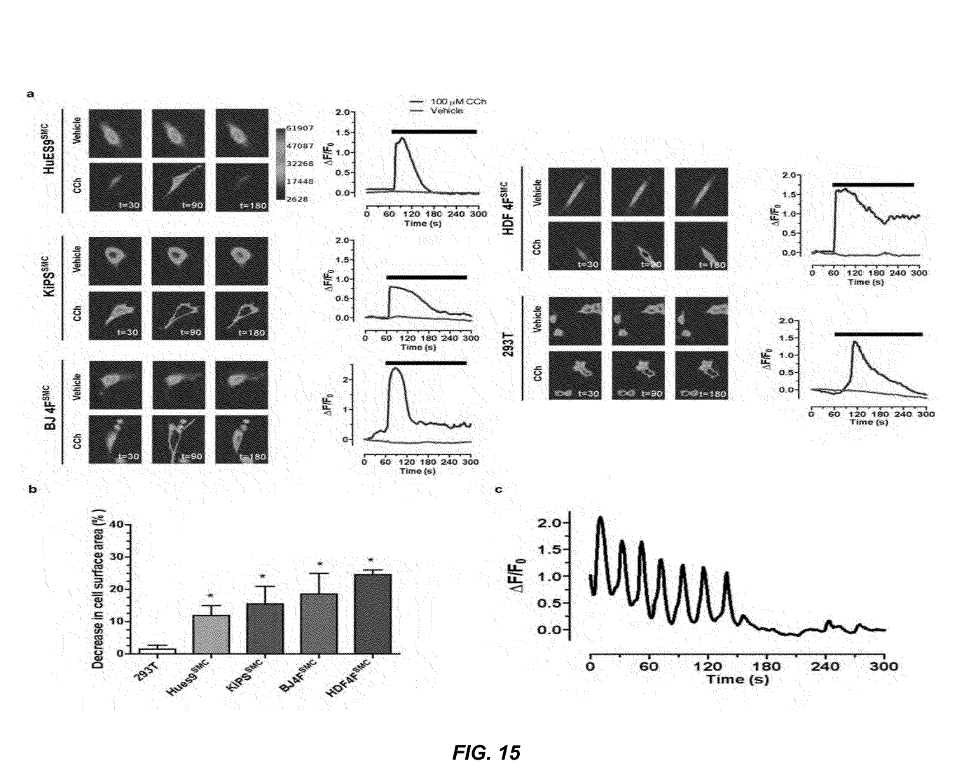

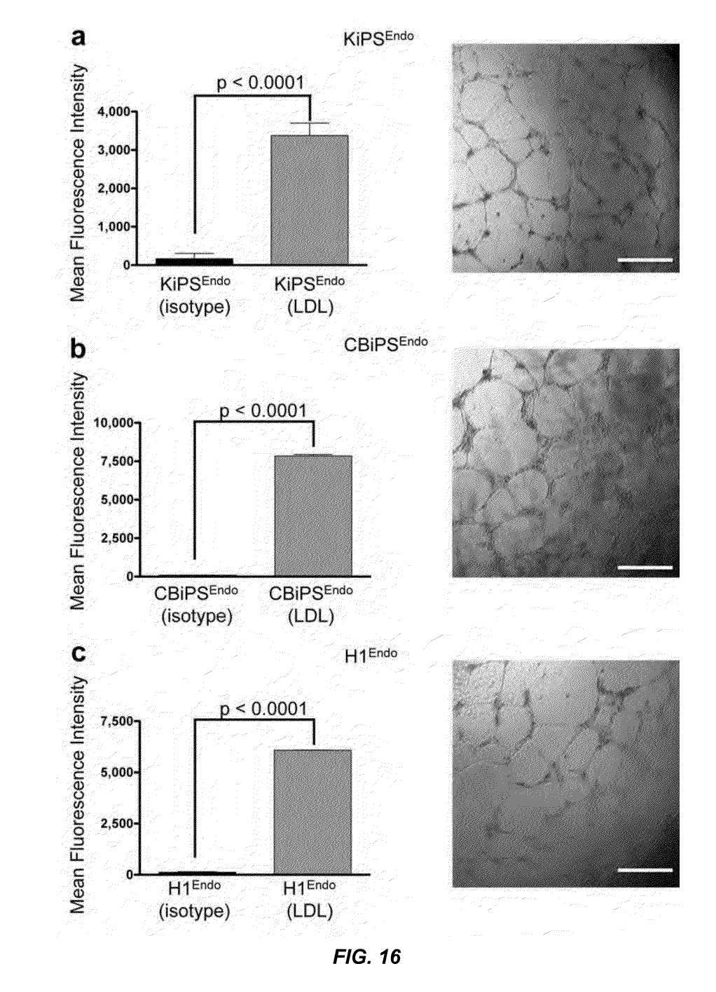

Applicants investigated the function of _{Fib}CD34+ cell-derived endothelial cells by measuring acetylated-LDL uptake, a characteristic of mature endothelial cells. The cells showed significantly higher rates of LDL uptake as compared to differentiated endothelial cells in the presence of control Alexa-488 used to measure unspecific fluorescence background (FIG. 3h and FIG. 16). The converted endothelial cells also aggregated into vessel-like structures in vitro and were able to form functional vessels, allowing for blood circulation, after 17 days in vivo upon subcutaneous implantation of a matrigel plug in which the endothelial cells were embedded (FIGS. 3i, 4 and FIG. 16), demonstrating connection of newly formed vessels to the pre-existing vasculature in vivo. After 17 days, endothelial cell identity, in the matrigel plugs extracted from the animals, was verified by Ulex-lectin binding and the human origin of the cells by in situ hybridization (FIGS. 4b,c) as well as with antibodies specific for human CD31 and human nuclear antigen (FIGS. 4d,e).

Applicants have established an efficient method for the conversion of neonatal and adult human fibroblasts into CD34+ angioblast-like progenitor cells. Applicants' approach couples the generation of plastic reprogramming intermediates with subsequent induction of an angioblast fate with chemically-defined MIM media. These angioblast-like cells could be further differentiated into functional endothelial and smooth muscle cells.

Applicants observed differences at both the transcriptome and methylome level when comparing Applicants' converted endothelial and smooth muscle cells to the primary cells used as positive controls. One reason could be that Applicants' generated cell populations include different sub-types of cells, as compared to the primary cells (Human Umbilical Vein Endothelial Cells and Arterial Smooth Muscle Cells). Alternatively, these differences could be reminiscent of experimental variation as seen between and within iPSC and ESC lines (Yamanaka, S. Cell Stem Cell 10, 678-684 (2012)). Lastly, residual epigenetic marks from the initial fibroblasts could also account for some observed differences (Vierbuchen, T. & Wernig, M., Nat Biotechnol 29, 892-907 (2011)). Furthermore, the fact that induction of “plasticity” relies on a first phase of epigenetic erasure, which by similarity with iPSC reprogramming might imply a stochastic process, strongly suggests that the heterogeneity observed at the molecular level during the conversion process might be due to the presence of cells with varying degrees of epigenomic plasticity. Nevertheless, all the cells generated (whether differentiated from PSCs or derived by conversion of human fibroblasts) demonstrated functional properties, highlighting the potential of these novel conversion methodologies as well as the importance of analyzing functional parameters in reprogramming paradigms (Vierbuchen, T. & Wernig, M., Nat Biotechnol 29, 892-907 (2011); Yamanaka, S. Cell Stem Cell 10, 678-684 (2012); Yusa, K., et al., Nature 478, 391-394 (2011)).

Conversion into angioblast-like progenitor cells occurred in the absence of detectable iPSC colony formation, surface marker expression and re-activation of the endogenous pluripotency transcription network, therefore shortening considerably the time required for generation of the desired cell types. Further, the lack of pluripotent marker expression and teratoma formation is indicative of a conversion process that does not result in typical iPSC features.

Different than a recent report describing the conversion of amniotic cells to endothelial cells (Ginsberg, M., et al., Cell 151, 559-575 (2012)), this study is the first demonstration that short-term induction by iPSC reprogramming conditions, followed by exposure to a chemically defined differentiation media, is sufficient for the conversion of neonatal and adult human fibroblasts into angioblast-like progenitor cells with multipotent differentiation potential. Of note, not only the autonomous effects of the reprogramming factors but the overall combination of stem cell culture conditions promoting cell proliferation, as exemplified by the requirement of bFGF, are crucial during the process of conversion. Interestingly, culture conditions have also been highlighted as a critical component during the conversion process in similar reports (Efe, J. A., et al., Nat Cell Biol 13, 215-222 (2011); Kim, J., et al., Proc Natl Acad Sci USA, 108, 7838-7843 (2011)).

Contrary to direct lineage conversion, which requires precise knowledge and screening of molecules defining target cell identity, induction of “plastic/de-differentiation” states coupled to specific differentiation protocols might provide a general, more readily accessible, platform towards the broader generation of clinically relevant cell types. Furthermore, whereas direct lineage conversion might be viewed as an “unnatural” process (Vierbuchen, T. & Wernig, M., Nat Biotechnol 29, 892-907 (2011)) occurring in the absence of progenitor cell generation, Applicants' results and those reported for the murine system (Efe, J. A., et al., Nat Cell Biol 13, 215-222 (2011); Kim, J., et al., Proc Natl Acad Sci USA, 108, 7838-7843 (2011)) show, as during normal embryogenesis, the formation of intermediate progenitor states. This may have two major practical implications. First, the generation of progenitor cells with multilineage differentiation capacity strongly diversifies the spectra of applications as opposed to direct lineage conversion. Second, the inability to generate proliferative populations by direct lineage conversion could represent a major limitation for applications in which large numbers of cells are required (Vierbuchen, T. & Wernig, M., Nat Biotechnol 29, 892-907 (2011); Sancho-Martinez, I. et al., Nat Cell Biol 14, 892-899 (2012)). In the case shown here, the conversion of human fibroblasts into vascular smooth muscle and endothelial cells proceeds through the generation of an expandable population of vascular progenitors with multilineage differentiation capacity.

Experimental Procedures

Reagents and Antibodies

The following antibodies were used at the specified concentrations: mouse anti-human CD34-APC 1:10 (130-046-703, Miltenyi), mouse anti-human CD133/2 (293C3)-PE 1:10 (130-090-853, Miltenyi), mouse anti-human CD144-PE 1:10 (VE-cadherin; 560410, BD biosciences), mouse anti-human CD144-APC 1:10 (VE-cadherin; 348507, Biolegend), CD105-PE 1:10 (endoglin; ab60902, Abeam), mouse anti-human CD105-PE 1:10 (endoglin; 560839, BD biosciences), CD31-FITC 1:10 (555445, BD biosciences), CD117-PeCy7 1:10 (c-Kit; 339195, BD biosciences), VEGFR2-PE 1:10 (KDR; 560494, BD biosciences), mouse anti-human CD45-FITC 1:10 (130-080-202, Miltenyi), anti-human CD235a-PE 1:10 (340947, BD biosciences), mouse APC isotype control 1:10 (555751, BD biosciences), mouse FITC isotype control 1:10 (555748, BD biosciences), PeCy7 isotype control 1:10 (557872, BD biosciences), PE isotype control 1:10 (555749, BD biosciences), VE-Cadherin 1:500 (555661, BD biosciences), Endoglin 1:500 (M3527, DAKO), Anti-von Willebrand Factor 1:200 (vWF; 7356, Millipore), calponin 1:500 (Dako, M3556), α-SMA 1:500 (AB56994, Abeam), α-SMA 1:1000 (A5228, Sigma), PECAM-1 (M−20) 1:100 (CD31; sc1506, Santa Cruz Biotechnology) anti-Human Nuclei 1:100 (MAB1281, Millipore), DAPI [5 mg ml⁻¹] 1:2000 (D1306, Invitrogen), Hoechst 33342 [5 mg ml⁻¹] 1:2000 (B2261, Sigma), Alexa fluor 488 goat anti-mouse (A11001, Invitrogen), Alexa fluor 488 Donkey anti-goat (A11055, Invitrogen), Alexa fluor 568 Donkey anti-mouse (A10037, Invitrogen), Alexa-fluor 568 Donkey anti-rabbit (A10042, Invitrogen).

Cell Culture

Human ES cells, H1 (WA1, WiCell), HuES 9 (On the Worldwide Web www.mcb.harvard.edu/melton/hues/) and Human iPS cells CBiPS (Giorgetti, A., et al., Cell Stem Cell 5, 353-357 (2009)) and KiPS (Aasen, T., et al., Nat Biotechnol 26, 1276-1284 (2008)) (KIPS 4F#2, CBiPS 2F#4) (passage 25-45) were cultured in chemically defined hES/hiPS growth media (mTeSR (Ludwig, T. E., et al., Nat Biotechnol 24, 185-187 (2006)) on growth factor reduced matrigel (35623, BD biosciences) coated plates). Briefly, 70-80% confluent hES/iPS cells were treated with dispase (Invitrogen) for 7 minutes at 37° C., colonies were dispersed to small clusters and lifted carefully using a 5 ml glass pipette, at a ratio of ˜1:4. Neonatal human fibroblasts (HFF-1, BJ; ATCC) and adult human dermal fibroblasts (HDF-693) were cultured in DMEM containing 10% FBS, 2 mM GlutaMAX (Invitrogen), 50 U ml⁻¹ penicillin and 50 mg ml⁻¹ streptomycin (Invitrogen). Human Umbilical Vein Endothelial Cells (HUVEC) were purchased from Promocell and cultured in EBM medium supplemented with EGM-2 singlequots (cc-3162, Lonza), 2% FBS, hEGF 10 μg ml⁻¹, Heparin 100 μg ml⁻¹ (Sigma). Mesodermal Induction Media (MIM) consists of DMEM:F12, 15 mg ml⁻¹ stem cell grade BSA (MP biomedicals), 17.5 μg ml⁻¹ Human Insulin (SAFC bioscience), 275 μg ml⁻¹ Human holo-transferrin (Sigma Aldrich), 20 ng ml⁻¹ bFGF (Stemgent), 50 ng ml⁻¹ Human VEGF-165 aa (Humanzyme), 25 ng ml⁻¹ human BMP4 (Stemgent), 450 μM monothioglycerol (Sigma Aldrich), 2.25 mM each L-Glutamine and Non-essential amino acids (Invitrogen). iPS/ES-derived endothelial cells were cultivated in EBM-2 medium supplemented with EGM-2 singleQuot kit (cc-3162, Lonza). iPS/ES-derived smooth muscle cells were cultured in SmBM medium supplemented with SmGM-2 singleQuot kit (cc-3182, Lonza). All the cells were grown in collagen I coated plates (BD biosciences). All cell lines were maintained in an incubator (37° C., 5% CO₂) with media changes every day (hES/iPS) or every second day (HUVEC/Fibroblasts).

Directed Differentiation of hES/hiPS Cells in Chemically Defined Conditions

Human ES/iPS cells cultured as described above were freshly split on matrigel-coated plates, making sure the sub-colonies were of small size (−300-500 cells/colony). After 24 hours of recovery in mTeSR, the cells were gently washed using DMEM:F12 (Invitrogen) and allowed to grow in chemically defined MIM differentiation media. Media was changed every second day with addition of half the volume of media every other day.

Single Cell Differentiation Assays

Upon MIM differentiation for 8 days, CD34+ Angioblast-like cells (Angioblast-like) were sorted and plated in collagen I coated 48-well plates at a density of one cell/well in either EBM-2 (endothelial differentiation) or SmBM (Smooth Muscle differentiation) supplemented as described above. After 7 days in the respective differentiation conditions cells were washed once with PBS and fixed with 4% Paraformaldehyde (PFA) in 1× PBS. Following fixation, cells were blocked and permeabilized for 1 hour at 37° C. with 5% BSA/5% appropriate serum/1× PBS in the presence of 0.1% Triton X100. Subsequently, cells were incubated overnight at 4° C. with an anti-endoglin antibody in case of cells in EBM-2/EGM-2 or with an anti-calponin antibody in the case of cells in SmBM/SmGM-2. Cells were then washed thrice with 1× PBS, incubated for 1 hour at 37° C. with the respective secondary antibodies and 20 minutes with DAPI for nuclear staining. Following incubation, cells were washed thrice with 1× PBS before microscopy analysis and scoring.

Conversion of Human Fibroblasts into Angioblast-Like CD34+ Progenitor Cells

For retroviral infection, 75,000/well human fibroblast cells (HFF-1, BJ, HFF-693) were plated on matrigel-coated 6-well plates. The next day, cells were infected with an equal ratio of either a combination of 4 pMX-derived retroviruses encoding Oct4, SOX2, KLF4 and c-Myc (4F) or 5 pMX-derived retroviruses encoding Oct4, SOX2, KLF4, c-Myc and miRs302-367 (4F/miR5). Scramble miRNA control (PMIRH000PA-1, SBI) was used whenever appropriate. The plates were infected by spinfection of the cells at 1850 rpm for 1 hour at room temperature in the presence of polybrene (4 μml⁻¹) and put back in the incubator without media change. 24 hours later, the media was switched to WiCell media composed of DMEM/F12 (Invitrogen), 20% Knockout serum replacement, 10 ng ml⁻¹ bFGF, 1 mM GlutaMax, 0.1 mM non-essential amino acids and 55 μM β-mercaptoethanol; with media changes every day. After 6 days, cells were split at a ratio of 1:3 on to matrigel coated 6-well plates supplemented with WiCell media for another 2 days. The cells were then washed once with DMEM/F12 and induced for differentiation for 8 days in the presence of MIM. Media was changed every second day with addition of half the volume of media every other day.

For episomal transfection, 2.10⁶ cells were transfected with 1.5 μg each of pCXLE-episomal vectors encoding for Oct4, SOX2, KLF4, LMYC, LIN28 and shRNA-p53 (Okita, K., et al., Nat Methods 8, 409-412 (2011)) (#27077, #27078 and #27080, addgene) with and without addition of pcDNA3.1 encoding for miRs302-367 (6F or 6F/miRs). Fibroblasts were transfected by nucleofection (Amaxa NHDF nucleofector kit, # VPD-1001) according to manufacturer's instructions, and plated back on to matrigel-coated wells. After 6 days resting in DMEM/F12 supplemented with 10% FBS, 0.1 mM non-essential amino acids and 2 mM GlutaMAX, the media was switched to WiCell media with media changes every day. After 6 days, cells were split at a ratio of 1:3 on to matrigel coated 6-well plates with WiCell media for another 2 days. The cells were then washed once with DMEM:F12 and induced for differentiation for 8 days in the presence of MIM. Media was changed every second day with addition of half the volume of media every other day.

RNA Isolation and Real Time-PCR Analysis

Total cellular RNA was isolated using Trizol Reagent (Invitrogen) according to the manufacturer's recommendations. 2 μg of DNAse1 (Invitrogen) treated total RNA was used for cDNA synthesis using the SuperScript II Reverse Transcriptase kit for RT-PCR (Invitrogen). Real-time PCR was performed using the SYBR-Green PCR Master mix (Applied Biosystems). The levels of expression of respective genes were normalized to corresponding GAPDH values and are shown as fold change relative to the value of the control sample. All the samples were done in triplicate. A list including precise mRNA fold-change quantifications of the qPCR data summarized in FIGS. 1-3 is provided in Table 1. The list of the primers used for real time-PCR experiments are listed in Table 2.

Flow Cytometry Analysis

Human ES/iPS cells undergoing directed differentiation, lineage converted CD34+ cells or their respectively derived endothelial cells were harvested using TripLE (Invitrogen), washed once with PBS and further incubated with the corresponding antibodies in the presence of FACS blocking buffer (1× PBS/10% FCS) for 1 hour on ice in the absence of light. After incubation, cells were washed thrice with 1 ml of FACS blocking buffer and resuspended in a total volume of 200 μl prior to analysis. A minimum of 10,000 cells in the living population were analyzed by using a BD LSRII flow cytometry machine equipped with 5 different lasers and the BD FACSDiva software. Percentages are presented after subtracting isotype background and referring to the total living population analyzed. Results are representative of at least three independent experiments with a minimum of two technical replicates per experiment.

Cell Sorting

After 8 days of differentiation CD34+ cells were stained as described above and sorted by using a BDAria II FACS sorter (BD Biosystems). Alternatively, CD34+ cells were enriched using anti-CD34 conjugated magnetic beads (Miltenyi) according to the manufacturer's instructions with slight modifications. Briefly, up to 10⁹ cells were incubated with constant mixing at 4° C. with 100 μl of the corresponding magnetic beads in the presence of 100 μl of Fc-blocking solution in a total volume of 500 μl FACS blocking buffer. After 1 hour, cells were sorted by two consecutive rounds of column separation in order to increase purity by applying MACS separation magnets. Shortly, cells were passed through the first MS separation column allowing binding of labeled cells. Non-labeled cells were washed thoroughly with 3 ml FACS blocking buffer prior to elution of the labeled fraction. Eluted labeled cells were then subjected to a second purification step as described above.

Differentiation of CD34+ Cells to Endothelial Cells.

PSC- and lineage converted-CD34+ cells, isolated by MACS or by FACS sorting after 8 days of differentiation in MIM, were plated in collagen I coated plates (50,000 cells well⁻¹ of a 12 well plate) and cultured in EBM-2/EGM-2 (Lonza) with media changes every day. After 5-8 days in culture, upon reaching 90% confluence, cells were split 1:4, using TripLE (Invitrogen). The cells were cultured for at least 8 passages.

Differentiation of CD34+ Cells to Smooth Muscle Cells.

PSC- and lineage converted-CD34+ cells, isolated by MACS or by FACS sorting after 8 days of differentiation in MIM, were plated in collagen I coated plates (50,000 cells well⁻¹ of a 12-well plate) and cultured in SmBM/SmGM-2 (Lonza) with media changes every day. After 5-8 days in culture, upon reaching 90% confluence, cells were split 1:4, using TripLE (Invitrogen). The cells were cultured for at least 8 passages.

DNA Methylation Analysis

Illumina 450K Infinium Methylation Arrays were normalized and pre-processed in Genome Studio. Probes with missing values were removed. A filter for average Beta value difference between groups (PSCs, Fibroblasts, primary human Arterial Smooth Muscle Cells (PriSMC), primary human Umbilical Vein Endothelial Cells (PriEC), PSC CD34+ Progenitor Cells, PSC Endothelial Cells (iECs), converted Endothelial Cells (cECs), PSCSmooth Muscle Cells (iSMCs), converted Smooth Muscle Cells (cSMCs)) of >0.3 was applied. The resulting probes were used for ANOVA analysis using R scripts with a p-value filter of <0.0001)(at this point, 30,000 probes remained). Probes with beta value difference of at least 0.3 ((max−min)>=0.3) were used. ANOVA test (p<0.05; Var 0.58) was applied to obtain statistically significant differentially methylated probes among the 5 groups in both SMC and EC sample group. The resulting probes were used for hierarchical clustering using Cluster 3.0 with complete linkage. Venn Diagram Plotter (http://omics.pnl.gov/softwareNennDiagramPlotter.php) was used to generate “area-proportional Venn Diagrams”. Datasets are available at the NCBI website, series reference GSE40927 (On the Worldwide Web at www.ncbi.nlm.nih.gov/geo/query/acc.cgi?acc=GSE40927).

Gene Expression Microarray Analysis

The following groups were analyzed: PSCs, Fibroblasts, primary human Arterial Smooth Muscle Cells (PriSMC), primary human Umbilical Vein Endothelial Cells (PriEC), PSC→Endothelial Cells (iECs), converted Endothelial Cells (cECs), PSC→Smooth Muscle Cells (iSMCs) and converted Smooth Muscle Cells (cSMCs). Briefly, total RNA was extracted from collected sample pellets (Ambion mirVana; Applied Biosystems) according to the manufacturer's protocol. RNA quantity (Qubit™ RNA BR Assay Kits; Invitrogen) and quality (RNA6000 Nano Kit; Agilent) was determined to be optimal for each sample prior to further processing. 200 ng RNA per sample was amplified using the Illumina® Total Prep™ RNA Amplification Kit according to manufacturer's protocol and quantified as above. 750 ng RNA/sample was hybridized to Illumina HT-12v3 Expression BeadChips, scanned with an Illumina iScan Bead Array Scanner and quality controlled in GenomeStudio and the lumi bioconductor package. All RNA processing and microarray hybridizations were performed in-house according to manufacturer's protocols. Differential expression was defined as a minimum 2× fold-change and multiple-testing corrected p<0.05 by ANOVA. The resulting probes were used for hierarchical clustering using Cluster 3.0 with complete linkage. Probes with minimum gene expression differences between groups of 2× fold-change were obtained. Venn Diagram Plotter (http://omics.pnl.gov/softwareNennDiagramPlotter.php) was used to generate “area-proportional Venn Diagrams”. Datasets are available at the NCBI website, series reference GSE40927 (on the WorlWide Web www.ncbi.nlm.nih.gov/geo/query/acc.cgi?acc=GSE40927).

Determination of Copy Number by Quantitative PCR.

Briefly, total DNA was extracted using the Qiagen DNeasy Blood & Tissue kit (QIAGEN). The purity and quantity of DNA was measured using a NanoDrop 8000 spectrophotometer (Thermo Scientific), and then used as templates for absolute quantitation by qPCR assay (Okita, K., et al., Nat Methods 8, 409-412 (2011)). The primers used are listed above and their amplification efficiencies, as well as specificity, were checked by performing standard curve and melting curve analyses.

Immunocytochemistry and Fluorescence Microscopy

Briefly, cells were washed thrice with PBS and fixed using 4% PFA in 1× PBS. After fixation, cells were blocked and permeabilized for 1 hour at 37° C. with 5% BSA/5% appropriate serum/1× PBS in the presence of 0.1% Triton X100. Subsequently, cells were incubated with the indicated primary antibody either for 1 hour at room temperature or overnight at 4° C. The cells were then washed thrice with 1× PBS and incubated for 1 hour at 37° C. with the respective secondary antibodies and 20 minutes with DAPI or Hoechst 33342. Cells were washed thrice with 1× PBS before analysis. Sections were analyzed by using an Olympus 1X51 upright microscope equipped with epifluorescence and TRITC, FITC, and DAPI filters. Confocal image acquisition was performed using a Zeiss LSM 780 laser scanning microscope (Carl Zeiss Jena, Germany) with 20×, 40× or 63× immersion objectives.

Acetylated-LDL Uptake Assay and Vascular Tube-Like Structure Formation Assay

In short, 80% confluent endothelial cells derived from human ES/iPS cells were incubated with 10 μml⁻¹-Dil-Ac-LDL (L23380, Molecular Probes) for 3 hours in DMEM:F12. The cells were washed 3 times with PBS, dissociated using TripLE and analyzed by flow cytometry.

Briefly, to assess the formation of capillary structures, a suspension of 4.10⁵ cells ml⁻¹ endothelial cells in the presence EBM-2/EGM-2 was prepared. Subsequently, 100 μl well⁻¹ were dispensed on flat bottom 96 well plates coated with Matrigel (BD biosciences). Tube formation was observed after 24 hours of incubation and a minimum of three replicates per experiment analyzed.

Hematopoietic Colony-Forming Assays

Hematopoietic clonogenic assays were performed in 35-mm low adherent plastic dishes (Stem Cell Technologies, Vancouver, BC, Canada) using 1.1 ml dish⁻¹ of methylcellulose semisolid medium (MethoCult H4434 classic, Stem Cell Technologies) according to the manufacturer's instructions. Briefly, enriched CD34+ cells were sorted and immediately plated at various densities: 1.5×10³ ml⁻¹, 3×10³ ml⁻¹ and 6×10³ ml⁻¹. All assays were performed in duplicate. After 21 days of incubation plates were analyzed for the presence of both, Colony-forming units (CFU) and Burst-forming units (BFU).

Animals

All murine experiments were conducted with approval of The Salk Institute Institutional Animal Care and Use Committee (IACUC). NOD.Cg-PrkdcscidI12rgtm1Wj1/SzJ mice (or NOD-Scid IL2rγnull abbreviated as NSG; age, 7 weeks; weight, 20 g) were purchased from Charles River Laboratories, housed in air-flow racks on a restricted access area and maintained on a 12 hour light/dark cycle at a constant temperature (22±1° C.).

Matrigel Plug Assay

Anaesthesia was induced using a mixture of Xylazine (Rompun® 2%, Bayer) at 10 mg kg⁻¹ and Ketamine (Imalgene1000, Merial) at 100 mg kg⁻¹ in NaCl at 0.9% i.p injected at a dose of 10 ml kg⁻¹. The animals' backs were shaved, swabbed with Hexomedine®. Prior injection, HUVECs, HUES9−, KiPS−, BJ 6F− and BJ 6F/miRs-derived endothelial cells were harvested using TripLE (invitrogen). A total of 1.10⁶ cells were resuspended in 500 μl of cold matrigel (Matrigel basement membrane matrix from BD Biosciences adjusted to 9.8 mg ml⁻¹ PBS) supplemented with 150 ng of bFGF. Cell and no cell containing matrigel solutions were then injected subcutaneously in the back of mice, carefully positioning the needle between the epidermis and the muscle layer. Seventeen days later, mice were sacrificed and the matrigel plugs were removed by a wide excision of the back skin, including the connective tissues (skin and all muscle layers).

Tissue Processing/Analyses

For immunohistochemistry (IHC), in situ hybridization (ISH) or immunofluorescence (IF) analysis, cell-containing implants with associated connective tissues were fixed with Accustain® (SIGMA) for 24 hours, dehydrated through an ethanol series and then processed for paraffin embedding before being sliced with a microtome. Slices from paraffin-embedded samples were stained with appropriate antibodies or probes. Alternatively, plugs were harvested and fixed with a 4% paraformaldehyde solution overnight at 4°, washed thrice in PBS and then incubated in a glucose solution (30%) for another 48 hours before being sliced (45 μm) with a cryostat (Leica). Both methodologies were equally successful to identify neovasculature derived from human cells.

For IHC, slides were stained with an anti human-CD31 monoclonal antibody and then incubated with biotin-labeled secondary antibody followed by incubation with streptavidin-HRP (Ventana Roche).

For ISH, slides were hybridized according to the manufacturer's protocol with an Alu probe (780-2845, Ventana Roche) and then labeled with the ISH iView Blue Detection kit (760-092, Ventana Roche).

For IHC and ISH, images were then captured with a camera mounted on a light microscope (Nikon E-800).

For immunofluorescence assays, slides were stained with either rhodamine-labeled Ulex Europaeus Agglutinin I (UEA I, a marker for human endothelial cells from Vector Laboratories) or PECAM-1 (M−20) (CD31; sc1506, Santa Cruz Biotechnology) and anti-Human Nuclei 1:100 (MAB1281, Millipore) counterstained with DAPI. Images were captured with confocal microscopes (Zeiss, LSM 510 or LSM780).

Calcium Live Cell Imaging.

Subconfluent cells were washed with DMEM:F12 and incubated for 45 minutes with 1 μM Fluo-4/AM (Molecular Probes) in 0.5% BSA, DMEM:F12 in an incubator at 37° C., 95% CO₂. After washing to remove unloaded dye, cells responses to 100 μM carbachol or vehicle (water) were imaged in HEPES-buffered, phenol red-free DMEM:F12 in a wide field fluorescent microscope (Olympus BX61WI) equipped for fast fluorescent imaging. Image capture was performed with Metamorph and an EM-CCD camera (Hamamatsu). Image analysis was carried out with Metamorph and Fiji software. To determine functional SMC contraction after stimulations, the cell surface area was determined before and after carbachol exposure.

Statistical Evaluation

Statistical analyses of all endpoints were performed by using standard unpaired Student t test (one-tailed, 95% confidence intervals) using the SPSS/PC+statistics 11.0 software (SPSS

Inc.). All data are presented as mean±standard deviation (s.d.) or standard error of the mean (s.e.m.) where indicated and represent a minimum of two independent experiments with at least two technical duplicates.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Specific mRNA fold-change of the qPCR data summarized in FIGS. 1-3.

| BJ | | | | | | | | |
|---|---|---|---|---|---|---|---|---|
| Gene | | | BJ +6FEndo | | | BJ +6F/miRsEndo | | |
| name | Mean | s.d. | Gene name | Mean | s.d. | Gene name | Mean | s.d. |
| ALK1 | 1 | 0.009428 | ALK1 | 340.5245 | 19.88818 | ALK1 | 86.38223 | 9.465531 |
| BMX | 1 | 0.077889 | BMX | 757.5554 | 47.16957 | BMX | 401.2288 | 23.43359 |
| CXCR4 | 1 | 0.046428 | CXCR4 | 16.61441 | 0.772781 | CXCR4 | 7.746201 | 0.679088 |
| EPHB2 | 1 | 0.171464 | EPHB2 | 204.4235 | 13.58666 | EPHB2 | 14.87017 | 0.794653 |
| CX 40 | 1 | 0.122565 | CX 40 | 108.0606 | 7.520833 | CX 40 | 84.65331 | 1.545456 |
| JAG1 | 1 | 0.024944 | JAG1 | 33.13132 | 0.532779 | JAG1 | 5.702519 | 0.247686 |
| NRP1 | 1 | 0.032998 | NRP1 | 32.71503 | 2.023558 | NRP1 | 11.24061 | 1.352172 |
| UNC5B | 1 | 0.08165 | UNC5B | 272.981 | 31.03569 | UNC5B | 34.44094 | 2.404126 |
| COUP- | 1 | 0.075865 | COUP-TFII | 865.7482 | 41.99166 | COUP- | 73.24854 | 3.902747 |
| TFII | | | | | | TFII | | |
| EPHB4 | 1 | 0.030912 | EPHB4 | 229.9045 | 13.16546 | EPHB4 | 67.53014 | 6.170527 |
| NRP2 | 1 | 0.044969 | NRP2 | 704.8953 | 36.43407 | NRP2 | 237.3611 | 10.57811 |
| PROX1 | 1 | 0.020332 | PROX1 | 26.32122 | 0.677874 | PROX1 | 37.43288 | 2.301246 |
| VEGFR3 | 1 | 0.033993 | VEGFR3 | 189.1548 | 16.15393 | VEGFR3 | 170.96 | 7.120987 |
| VWF | 1 | 0.148174 | VWF | 335.5906 | 11.36438 | VWF | 644.6964 | 45.7541 |
| VE- | 1 | 0.021602 | VE-cadherin | 1710.37 | 23.71019 | VE- | 2228.006 | 125.1184 |
| cadherin | | | | | | cadherin | | |

TABLE 2
The list of the primers used for real time-PCR experiments.

| Gene | 5′ oligo | 3′ Oligo |
|---|---|---|
| Oct4 | GGGTTTTTGGGATTAAGTTCTTCA | GCCCCCACCCTTTGTGTT |
| | (SEQ ID NO: 7) | (SEQ ID NO: 8) |
| NANOG | ACAACTGGCCGAAGAATAGCA | GGTTCCCAGTCGGGTTCAC |
| | (SEQ ID NO: 9) | (SEQ ID NO: 10) |
| SOX2 | CAAAAATGGCCATGCAGGTT | AGTTGGGATCGAACAAAAGCTATT (SEQ |
| | (SEQ ID NO: 11) | ID NO: 12) |
| T | GCCCTCTCCCTCCCCTCCACGCACAG | CGGCGCCGTTGCTCACAGACCACAGG |
| | (SEQ ID NO: 13) | (SEQ ID NO: 14) |
| TIE2 | TGCCACCCTGGTTTTTACGG | TTGGAAGCGATCACACATCTC |
| | (SEQ ID NO: 15) | (SEQ ID NO: 16) |
| CD31 | AACAGTGTTGACATGAAGAGCC | TGTAAAACAGCACGTCATCCTT |
| | (SEQ ID NO: 17) | (SEQ ID NO: 18) |
| GATA4 | ACACCCCAATCTCGATATGTTTG | GTTGCACAGATAGTGACCCGT |
| | (SEQ ID NO: 19) | (SEQ ID NO: 20) |
| HOXB4 | GTGAGCACGGTAAACCCCAAT | CGAGCGGATCTTGGTGTTG |
| | (SEQ ID NO: 21) | (SEQ ID NO: 22) |
| CD34 | CCTAAGTGACATCAAGGCAGAA | GCAAGGAGCAGGGAGCATA |
| | (SEQ ID NO: 23) | (SEQ ID NO: 24) |
| CD45 | ACAGCCAGCACCTTTCCTAC | GTGCAGGTAAGGCAGCAGA |
| | (SEQ ID NO: 25) | (SEQ ID NO: 26) |
| ANGPT1 | GGGGGAGGTTGGACTGTAAT | AGGGCACATTTGCACATACA |
| | (SEQ ID NO: 27) | (SEQ ID NO: 28) |
| ANGPT2 | GGATCTGGGGAGAGAGGAAC | CTCTGCACCGAGTCATCGTA |
| | (SEQ ID NO: 29) | (SEQ ID NO: 30) |
| HOXB4 | GTGAGCACGGTAAACCCCAAT | CGAGCGGATCTTGGTGTTG |
| | (SEQ ID NO: 31) | (SEQ ID NO: 32) |
| GATA1 | AGAAGCGCCTGATTGTCAGTA | AGAGACTTGGGTTGTCCAGAA |
| | (SEQ ID NO: 33) | (SEQ ID NO: 34) |
| GATA2 | GGCCCACTCTCTGTGTACC | CATCTTCATGCTCTCCGTCAG |
| | (SEQ ID NO: 35) | (SEQ ID NO: 36) |
| RUNX1 | AGAACCTCGAAGACATCGGC | GGCTGAGGGTTAAAGGCAGTG |
| | (SEQ ID NO: 37) | (SEQ ID NO: 38) |
| CXCR4 | CACCGCATCTGGAGAACCA | GCCCATTTCCTCGGTGTAGTT |
| | (SEQ ID NO: 39) | (SEQ ID NO: 40) |
| LMO2 | GGACCCTTCAGAGGAACCAGT | GGCCCAGTTTGTAGTAGAGGC |
| | (SEQ ID NO: 41) | (SEQ ID NO: 42) |
| TAL1 | CAAAGTTGTGCGGCGTATCTT | TCATTCTTGCTGAGCTTCTTGTC |
| | (SEQ ID NO: 43) | (SEQ ID NO: 44) |
| TEL | AAACTTCATCCGATGGGAGGA | CGCAGGGCTCTGGACATTTT |
| | (SEQ ID NO: 45) | (SEQ ID NO: 46) |
| FOXA1 | CCAAGGCCGCCTTACTCCTACA | CGCAGATGAAGACGCTTGGAGA |
| | (SEQ ID NO: 47) | (SEQ ID NO: 48) |
| AC133 | CATCCACAGATGCTCCTAAGGC | AAGAGAATGCCAATGGGTCCA |
| | (SEQ ID NO: 49) | (SEQ ID NO: 50) |
| ETV2 | CCGACGGCGATACCTACTG | GTTCGGAGCAAACGGTGAG |
| | (SEQ ID NO: 51) | (SEQ ID NO: 52) |
| TUBB3 | CCTGGAACCCGGAACCAT | AGGCCTGAAGAGATGTCCAAAG |
| | (SEQ ID NO: 53) | (SEQ ID NO: 54) |
| ACTA2 | CAGGGCTGTTTTCCCATCCAT | GCCATGTTCTATCGGGTACTTC |
| | (SEQ ID NO: 55) | (SEQ ID NO: 56) |
| CNN1 | GAGTCAACCCAAAATTGGCAC | GGACTGCACCTGTGTATGGT |
| | (SEQ ID NO: 57) | (SEQ ID NO: 58) |
| SMMHC | GGACGACCTGGTTGTTGATT | GTAGCTGCTTGATGGCTTCC |
| | (SEQ ID NO: 59) | (SEQ ID NO: 60) |
| CALD1 | AACAACCTGAAAGCCAGGAGG | GCTGCTTGTTACGTTTCTGC |
| | (SEQ ID NO: 61) | (SEQ ID NO: 62) |
| SM22- | CGCGAAGTGCAGTCCAAAATCG | GGGCTGGTTCTTCTTCAATGGGG |
| alpha | (SEQ ID NO: 63) | (SEQ ID NO: 64) |
| VE- | GACCGGGAGAATATCTCAGAGT | CATTGAACAACCGATGCGTGA |
| cadherin | (SEQ ID NO: 65) | (SEQ ID NO: 66) |
| endoglin | CCCACAAGTCTTGCAGAAACA | CTGGCTAGTGGTATATGTCACCT |
| | (SEQ ID NO: 67) | (SEQ ID NO: 68) |
| VWF | ATGTTGTGGGAGATGTTTGC | GCAGATAAGAGCTCAGCCTT |
| | (SEQ ID NO: 69) | (SEQ ID NO: 70) |
| GAPDH | GGACTCATGACCACAGTCCATGCC | TCAGGGATGACCTTGCCCACAG |
| | (SEQ ID NO: 71) | (SEQ ID NO: 72) |
| GBX2 | GACGAGTCAAAGGTGGAAGAC | GATTGTCATCCGAGCTGTAGTC |
| | (SEQ ID NO: 73) | (SEQ ID NO: 74) |
| OLIG3 | CTGTCGGAGCAGGACCTACA | GCGTCCGTTGATCTTCAGC |
| | (SEQ ID NO: 75) | (SEQ ID NO: 76) |
| MESP1 | AGCTTGGGTGCCTCCTTATT | TGCTTCCCTGAAAGACATCA |
| | (SEQ ID NO: 77) | (SEQ ID NO: 78) |
| NKX2-5 | CAAGTGTGCGTCTGCCTTT | CAGCTCTTTCTTTTCGGCTCTA |
| | (SEQ ID NO: 79) | (SEQ ID NO: 80) |
| ISL1 | AGATTATATCAGGTTGTACGGGATCA | ACACAGCGGAAACACTCGAT |
| | (SEQ ID NO: 81) | (SEQ ID NO: 82) |
| TBX6 | AGCCTGTGTCTTTCCATCGT | GCTGCCCGAACTAGGTGTAT |
| | (SEQ ID NO: 83) | (SEQ ID NO: 84) |
| MEOX1 | AAAGTGTCCCCTGCATTCTG | CACTCCAGGGTTCCACATCT |
| | (SEQ ID NO: 85) | (SEQ ID NO: 86) |
| TCF15 | GCACCTTCTGCCTCAGCAACCAGC | GGTCCCCCGGTCCCTACACAA |
| | (SEQ ID NO: 87) | (SEQ ID NO: 88) |
| PAX1 | CACACTCGGTCAGCAACATC | GGTTTCTCTAGCCCATTCACTG |
| | (SEQ ID NO: 89) | (SEQ ID NO: 90) |
| ALK1 | CGAGGGATGAACAGTCCTGG | GTCATGTCTGAGGCGATGAAG |
| | (SEQ ID NO: 91) | (SEQ ID NO: 92) |
| BMX | TACCTGAGGAGTCACGGAAAA | TTCACAGACATCGTAGCACATTT |
| | (SEQ ID NO: 93) | (SEQ ID NO: 94) |
| CX 40 | TGCAAGAGTGTGCTAGAGGC | ACAAAGCAGTCCACGAGGTAG |
| | (SEQ ID NO: 95) | (SEQ ID NO: 96) |
| CXCR4 | TGACGGACAAGTACAGGCTG | AGGGAAGCGTGATGACAAAGA |
| | (SEQ ID NO: 97) | (SEQ ID NO: 98) |
| EPHB2 | AAGGACTGGTACTATACCCACAG | TGTCTGCTTGGTCTTTATCAACC |
| | (SEQ ID NO: 99) | (SEQ ID NO: 100) |
| JAG1 | GGGGCAACACCTTCAACCTC | CCAGGCGAAACTGAAAGGC |
| | (SEQ ID NO: 101) | (SEQ ID NO: 102) |
| NRP1 | ACCCAAGTGAAAAATGCGAATG | CCTCCAAATCGAAGTGAGGGTT |
| | (SEQ ID NO: 103) | (SEQ ID NO: 104) |
| UNC5B | ACTGCCGTGACTTCGACAC | GCCTTGCCGTCTTAAAGTTGA |
| | (SEQ ID NO: 105) | (SEQ ID NO: 106) |
| COUP- | CGGATCTTCCAAGAGCAAGTG | ACAGGCATCTGAGGTGAACAG |
| TFII | (SEQ ID NO: 107) | (SEQ ID NO: 108) |
| EPHB4 | CCACCGGGAAGGTGAATGTC | CTGGGCGCACTTTTTGTAGAA |
| | (SEQ ID NO: 109) | (SEQ ID NO: 110) |
| NRP2 | AACTGCGAGTGGATTGTTTACG | TCTCGATTTCAAAGTGAGGGTTG |
| | (SEQ ID NO: 111) | (SEQ ID NO: 112) |
| PROX1 | GGCGAACTCGTATGAAGATGC | CTGGGAAATTATGGTTGCTCCT |
| | (SEQ ID NO: 113) | (SEQ ID NO: 114) |
| VEGFR3 | CTGGACCGAGTTTGTGGAGG | GTCACATAGAAGTAGATGAGCCG |
| | (SEQ ID NO: 115) | (SEQ ID NO: 116) |
| CD36 | AAGCCAGGTATTGCAGTTCTTT | GCATTTGCTGATGTCTAGCACA |
| | (SEQ ID NO: 117) | (SEQ ID NO: 118) |
| DES | GACGTGGATGCAGCTACTCTA | AGAGATTCAATTCTGCGCTCC |
| | (SEQ ID NO: 119) | (SEQ ID NO: 120) |
| NG2 | CCCTAGAGGTCCCCTATGGG | TGGCAGAAAACTCTTCAGCAC |
| | (SEQ ID NO: 121) | (SEQ ID NO: 122) |
| SMTN(A) | CAGTCCACCACCAGCATCTTCCAGT | AGCCAAGACTCGCGGCTACGA |
| | (SEQ ID NO: 123) | (SEQ ID NO: 124) |
| SMTN(B) | CGGCTGCGCGTGTCTAATCC | CTGTGACCTCCAGCAGCTTCCG |
| | (SEQ ID NO: 125) | (SEQ ID NO: 126) |
| VCL | ATGGGTCAAGGGGCATCCT | GGCCCAAGATTCTTTGTGTAAGT |
| | (SEQ ID NO: 127) | (SEQ ID NO: 128) |

### F. EMBODIMENTS

### Embodiment 1

A method of generating a vascular progenitor cell, said method comprising:

(i) transfecting a primary cell with a non-integrative reprogramming nucleic acid, thereby forming a transfected primary cell;
(ii) allowing said transfected primary cell to grow, thereby forming a multipotent cell; and
(iii) culturing said multipotent cell in a solution comprising BMP4, bFGF, and VEGF, thereby generating said vascular progenitor cell.

### Embodiment 2

The method of embodiment 1, wherein said primary cell is a human fibroblast.

### Embodiment 3

The method of embodiment 1, wherein said non-integrative reprogramming nucleic acid encodes a Sox-2 polypeptide, a Oct-4 polypeptide, a Klf-4 polypeptide, a cMyc polypeptide, a Lin28 polypeptide or a p53 siRNA.

### Embodiment 4

The method of embodiment 1, wherein said allowing to grow in step (ii) is performed in the absence of feeder cells.

### Embodiment 5

The method of embodiment 1, wherein said allowing to grow in step (ii) is performed for less than 10 days.

### Embodiment 6

The method of embodiment 5, wherein said allowing to grow in step (ii) is performed for about 8 days.

### Embodiment 7

The method of embodiment 1, wherein said multipotent cell does not express a pluripotency marker.

### Embodiment 8

The method of embodiment 7, wherein said pluripotency marker is a TRA1-81 polypeptide or a TRA1-60 polypeptide.

### Embodiment 9

The method of embodiment 1, wherein said multipotent cell is not capable of forming an embryonic stem cell colony.

### Embodiment 10

The method of embodiment 1, wherein said multipotent cell is not capable of teratoma formation.

### Embodiment 11

The method of embodiment 1, wherein said culturing said multipotent cell in step (iii) is performed for less than 10 days.

### Embodiment 12

The method of embodiment 11, wherein said culturing said multipotent cell in step (iii) is performed for about 8 days.

### Embodiment 13

The method of embodiment 1, wherein said vascular progenitor cell is capable of forming a smooth muscle cell or an endothelial cell.

### Embodiment 14

The method of any one of embodiments 1-13, wherein said vascular progenitor cell is a CD34⁺ cell.

### Embodiment 15

The method of any one of embodiments 1-13, wherein said method is performed with a plurality of primary cells, to form a population of cells comprising a plurality of vascular progenitor cells.

### Embodiment 16

The method of embodiment 15, further comprising separating said vascular progenitor cells from said population of cells.

### Embodiment 17

The method of embodiment 16, further comprising culturing said separated vascular progenitor cells in endothelial cell growth media to generate endothelial cells.

### Embodiment 18

The method of embodiment 16, further comprising culturing said separated vascular progenitor cells in smooth muscle cell growth media to generate smooth muscle cells.

### Embodiment 19

A method of generating a vascular progenitor cell, said method comprising culturing a multipotent cell in a solution comprising BMP4, bFGF, and VEGF, thereby generating said vascular progenitor cell.

### Embodiment 20

An isolated multipotent cell formed by a process comprising:

(i) transfecting a primary cell with a non-integrative reprogramming nucleic acid, thereby forming a transfected primary cell; and
(ii) allowing said transfected primary cell to grow, thereby forming said multipotent cell.

### Embodiment 21

The isolated multipotent cell of embodiment 20, wherein said allowing to grow in step (ii) is performed in the absence of feeder cells.

### Embodiment 22

The isolated multipotent cell of embodiment 20, wherein said allowing to grow in step (ii) is performed for less than 10 days.

### Embodiment 23

The isolated multipotent cell of embodiment 22, wherein said allowing to grow in step (ii) is performed for about 8 days.

### Embodiment 24

The isolated multipotent cell of embodiment 20, wherein said multipotent cell does not express a pluripotency marker.

### Embodiment 25

The isolated multipotent cell of embodiment 24, wherein said pluripotency marker is a TRA1-81 polypeptide or a TRA1-60 polypeptide.

### Embodiment 26

The isolated multipotent cell of embodiment 20, wherein said multipotent cell is not capable of forming an embryonic stem cell colony.

### Embodiment 27

The isolated multipotent cell of embodiment 20, wherein said multipotent cell is not capable of teratoma formation.

### Embodiment 28

An isolated vascular progenitor cell formed by a process comprising:

(i) transfecting a primary cell with a non-integrative reprogramming nucleic acid, thereby forming a transfected primary cell;
(ii) allowing said transfected primary cell to grow, thereby forming a multipotent cell; and
(iii) culturing said multipotent cell in a solution comprising BMP4, bFGF, and VEGF, thereby forming said vascular progenitor cell.

### Embodiment 29

A method of generating an endothelial cell, said method comprising:

(i) transfecting a primary cell with a non-integrative reprogramming nucleic acid, thereby forming a transfected primary cell;
(ii) allowing said transfected primary cell to grow, thereby forming a multipotent cell;
(iii) culturing said multipotent cell in a solution comprising BMP4, bFGF, and VEGF, thereby generating said vascular progenitor cell; and
(iv) culturing said vascular progenitor cell in endothelial cell growth media, thereby forming said endothelial cell.

### Embodiment 30

The method of embodiment 29, wherein said vascular progenitor cell is a CD34⁺ cell.

### Embodiment 31

An isolated endothelial cell formed according to the method of embodiment 29 or 30.

### Embodiment 32

A method of generating a smooth muscle cell, said method comprising:

(i) transfecting a primary cell with a non-integrative reprogramming nucleic acid, thereby forming a transfected primary cell;
(ii) allowing said transfected primary cell to grow, thereby forming a multipotent cell;
(iii) culturing said multipotent cell in a solution comprising BMP4, bFGF, and VEGF, thereby generating said vascular progenitor cell; and
(iv) culturing said vascular progenitor cell in smooth muscle cell growth media, thereby forming said smooth muscle cell.

### Embodiment 33

The method of embodiment 32, wherein said vascular progenitor cell is a CD34⁺ cell.

### Embodiment 34

An isolated smooth muscle cell generated according to the method of embodiment 32 or 33.

What is claimed is:

1. A method of generating a vascular progenitor cell, said method comprising:
(i) transfecting a primary cell with a non-integrative reprogramming nucleic acid, thereby forming a transfected primary cell;
(ii) allowing said transfected primary cell to grow, thereby forming a multipotent cell; and
(iii) culturing said multipotent cell in a solution comprising BMP4, bFGF, and VEGF, thereby generating said vascular progenitor cell.

2. The method of claim 1, wherein said non-integrative reprogramming nucleic acid encodes a Sox-2 polypeptide, a Oct-4 polypeptide, a Klf-4 polypeptide, a cMyc polypeptide, a Lin28 polypeptide or a p53 siRNA.

3. The method of claim 1, wherein said allowing to grow in step (ii) is performed for less than 10 days.

4. The method of claim 1, wherein said multipotent cell does not express a pluripotency marker.

5. The method of claim 4, wherein said pluripotency marker is a TRA1-81 polypeptide or a TRA1-60 polypeptide.

6. The method of claim 1, wherein said multipotent cell is not capable of forming an embryonic stem cell colony.

7. The method of claim 1, wherein said multipotent cell is not capable of teratoma formation.

8. The method of claim 1, wherein said culturing said multipotent cell in step (iii) is performed for less than 10 days.

9. The method of claim 1, wherein said vascular progenitor cell is capable of forming a smooth muscle cell or an endothelial cell.

10. An isolated multipotent cell formed by a process comprising:
(i) transfecting a primary cell with a non-integrative reprogramming nucleic acid, thereby forming a transfected primary cell; and
(ii) allowing said transfected primary cell to grow, thereby forming said multipotent cell.

11. The isolated multipotent cell of claim 10, wherein said allowing to grow in step (ii) is performed for less than 10 days.

12. The isolated multipotent cell of claim 10, wherein said multipotent cell does not express a pluripotency marker.

13. The isolated multipotent cell of claim 12, wherein said pluripotency marker is a TRA1-81 polypeptide or a TRA1-60 polypeptide.

14. The isolated multipotent cell of claim 10, wherein said multipotent cell is not capable of forming an embryonic stem cell colony.

15. The isolated multipotent cell of claim 10, wherein said multipotent cell is not capable of teratoma formation.

16. An isolated vascular progenitor cell formed by a process comprising:
(i) transfecting a primary cell with a non-integrative reprogramming nucleic acid, thereby forming a transfected primary cell;
(ii) allowing said transfected primary cell to grow, thereby forming a multipotent cell; and
(iii) culturing said multipotent cell in a solution comprising BMP4, bFGF, and VEGF, thereby forming said vascular progenitor cell.

17. A method of generating an endothelial cell, said method comprising:
(i) transfecting a primary cell with a non-integrative reprogramming nucleic acid, thereby forming a transfected primary cell;
(ii) allowing said transfected primary cell to grow, thereby forming a multipotent cell;
(iii) culturing said multipotent cell in a solution comprising BMP4, bFGF, and VEGF, thereby generating said vascular progenitor cell; and
(iv) culturing said vascular progenitor cell in endothelial cell growth media, thereby forming said endothelial cell.

18. An isolated endothelial cell formed according to the method of claim 17.

19. A method of generating a smooth muscle cell, said method comprising:
(i) transfecting a primary cell with a non-integrative reprogramming nucleic acid, thereby forming a transfected primary cell;
(ii) allowing said transfected primary cell to grow, thereby forming a multipotent cell;
(iii) culturing said multipotent cell in a solution comprising BMP4, bFGF, and VEGF, thereby generating said vascular progenitor cell; and
(iv) culturing said vascular progenitor cell in smooth muscle cell growth media, thereby forming said smooth muscle cell.

20. An isolated smooth muscle cell generated according to the method of claim 19.


SEQUENCE LISTING (132 sequences)

SEQ ID NO: 1 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 2 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - protein, 190 aa, Homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 4 - protein, 190 aa, Homo sapiens; identical to SEQ ID NO: 3

SEQ ID NO: 5 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 6 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 7 - DNA, 24 bp, Artificial Sequence
  Synthetic polynucleotide
gggtttttgggattaagttcttca

SEQ ID NO: 8 - DNA, 18 bp, Artificial Sequence
  Synthetic polynucleotide
gcccccaccctttgtgtt

SEQ ID NO: 9 - DNA, 21 bp, Artificial Sequence
  Synthetic polynucleotide
acaactggccgaagaatagca

SEQ ID NO: 10 - DNA, 19 bp, Artificial Sequence
  Synthetic polynucleotide
ggttcccagtcgggttcac

SEQ ID NO: 11 - DNA, 20 bp, Artificial Sequence
  Synthetic polynucleotide
caaaaatggccatgcaggtt

SEQ ID NO: 12 - DNA, 24 bp, Artificial Sequence
  Synthetic polynucleotide
agttgggatcgaacaaaagctatt

SEQ ID NO: 13 - DNA, 26 bp, Artificial Sequence
  Synthetic polynucleotide
gccctctccctcccctccacgcacag

SEQ ID NO: 14 - DNA, 26 bp, Artificial Sequence
  Synthetic polynucleotide
cggcgccgttgctcacagaccacagg

SEQ ID NO: 15 - DNA, 20 bp, Artificial Sequence
  Synthetic polynucleotide
tgccaccctggtttttacgg

SEQ ID NO: 16 - DNA, 21 bp, Artificial Sequence
  Synthetic polynucleotide
ttggaagcgatcacacatctc

SEQ ID NO: 17 - DNA, 22 bp, Artificial Sequence
  Synthetic polynucleotide
aacagtgttgacatgaagagcc

SEQ ID NO: 18 - DNA, 22 bp, Artificial Sequence
  Synthetic polynucleotide
tgtaaaacagcacgtcatcctt

SEQ ID NO: 19 - DNA, 23 bp, Artificial Sequence
  Synthetic polynucleotide
acaccccaatctcgatatgtttg

SEQ ID NO: 20 - DNA, 21 bp, Artificial Sequence
  Synthetic polynucleotide
gttgcacagatagtgacccgt

SEQ ID NO: 21 - DNA, 21 bp, Artificial Sequence
  Synthetic polynucleotide
gtgagcacggtaaaccccaat

SEQ ID NO: 22 - DNA, 19 bp, Artificial Sequence
  Synthetic polynucleotide
cgagcggatcttggtgttg

SEQ ID NO: 23 - DNA, 22 bp, Artificial Sequence
  Synthetic polynucleotide
cctaagtgacatcaaggcagaa

SEQ ID NO: 24 - DNA, 19 bp, Artificial Sequence
  Synthetic polynucleotide
gcaaggagcagggagcata

SEQ ID NO: 25 - DNA, 20 bp, Artificial Sequence
  Synthetic polynucleotide
acagccagcacctttcctac

SEQ ID NO: 26 - DNA, 19 bp, Artificial Sequence
  Synthetic polynucleotide
gtgcaggtaaggcagcaga

SEQ ID NO: 27 - DNA, 20 bp, Artificial Sequence
  Synthetic polynucleotide
gggggaggttggactgtaat

SEQ ID NO: 28 - DNA, 20 bp, Artificial Sequence
  Synthetic polynucleotide
agggcacatttgcacataca

SEQ ID NO: 29 - DNA, 20 bp, Artificial Sequence
  Synthetic polynucleotide
ggatctggggagagaggaac

SEQ ID NO: 30 - DNA, 20 bp, Artificial Sequence
  Synthetic polynucleotide
ctctgcaccgagtcatcgta

SEQ ID NO: 31 - DNA, 21 bp, Artificial Sequence
  Synthetic polynucleotide
gtgagcacggtaaaccccaat

SEQ ID NO: 32 - DNA, 19 bp, Artificial Sequence
  Synthetic polynucleotide
cgagcggatcttggtgttg

SEQ ID NO: 33 - DNA, 21 bp, Artificial Sequence
  Synthetic polynucleotide
agaagcgcctgattgtcagta

SEQ ID NO: 34 - DNA, 21 bp, Artificial Sequence
  Synthetic polynucleotide
agagacttgggttgtccagaa

SEQ ID NO: 35 - DNA, 19 bp, Artificial Sequence
  Synthetic polynucleotide
ggcccactctctgtgtacc

SEQ ID NO: 36 - DNA, 21 bp, Artificial Sequence
  Synthetic polynucleotide
catcttcatgctctccgtcag

SEQ ID NO: 37 - DNA, 20 bp, Artificial Sequence
  Synthetic polynucleotide
agaacctcgaagacatcggc

SEQ ID NO: 38 - DNA, 21 bp, Artificial Sequence
  Synthetic polynucleotide
ggctgagggttaaaggcagtg

SEQ ID NO: 39 - DNA, 19 bp, Artificial Sequence
  Synthetic polynucleotide
caccgcatctggagaacca

SEQ ID NO: 40 - DNA, 21 bp, Artificial Sequence
  Synthetic polynucleotide
gcccatttcctcggtgtagtt

SEQ ID NO: 41 - DNA, 21 bp, Artificial Sequence
  Synthetic polynucleotide
ggacccttcagaggaaccagt

SEQ ID NO: 42 - DNA, 21 bp, Artificial Sequence
  Synthetic polynucleotide
ggcccagtttgtagtagaggc

SEQ ID NO: 43 - DNA, 21 bp, Artificial Sequence
  Synthetic polynucleotide
caaagttgtgcggcgtatctt

SEQ ID NO: 44 - DNA, 23 bp, Artificial Sequence
  Synthetic polynucleotide
tcattcttgctgagcttcttgtc

SEQ ID NO: 45 - DNA, 21 bp, Artificial Sequence
  Synthetic polynucleotide
aaacttcatccgatgggagga

SEQ ID NO: 46 - DNA, 20 bp, Artificial Sequence
  Synthetic polynucleotide
cgcagggctctggacatttt

SEQ ID NO: 47 - DNA, 22 bp, Artificial Sequence
  Synthetic polynucleotide
ccaaggccgccttactcctaca

SEQ ID NO: 48 - DNA, 22 bp, Artificial Sequence
  Synthetic polynucleotide
cgcagatgaagacgcttggaga

SEQ ID NO: 49 - DNA, 22 bp, Artificial Sequence
  Synthetic polynucleotide
catccacagatgctcctaaggc

SEQ ID NO: 50 - DNA, 21 bp, Artificial Sequence
  Synthetic polynucleotide
aagagaatgccaatgggtcca

SEQ ID NO: 51 - DNA, 19 bp, Artificial Sequence
  Synthetic polynucleotide
ccgacggcgatacctactg

SEQ ID NO: 52 - DNA, 19 bp, Artificial Sequence
  Synthetic polynucleotide
gttcggagcaaacggtgag

SEQ ID NO: 53 - DNA, 18 bp, Artificial Sequence
  Synthetic polynucleotide
cctggaacccggaaccat

SEQ ID NO: 54 - DNA, 22 bp, Artificial Sequence
  Synthetic polynucleotide
aggcctgaagagatgtccaaag

SEQ ID NO: 55 - DNA, 21 bp, Artificial Sequence
  Synthetic polynucleotide
cagggctgttttcccatccat

SEQ ID NO: 56 - DNA, 22 bp, Artificial Sequence
  Synthetic polynucleotide
gccatgttctatcgggtacttc

SEQ ID NO: 57 - DNA, 21 bp, Artificial Sequence
  Synthetic polynucleotide
gagtcaacccaaaattggcac

SEQ ID NO: 58 - DNA, 20 bp, Artificial Sequence
  Synthetic polynucleotide
ggactgcacctgtgtatggt

SEQ ID NO: 59 - DNA, 20 bp, Artificial Sequence
  Synthetic polynucleotide
ggacgacctggttgttgatt

SEQ ID NO: 60 - DNA, 20 bp, Artificial Sequence
  Synthetic polynucleotide
gtagctgcttgatggcttcc

SEQ ID NO: 61 - DNA, 21 bp, Artificial Sequence
  Synthetic polynucleotide
aacaacctgaaagccaggagg

SEQ ID NO: 62 - DNA, 20 bp, Artificial Sequence
  Synthetic polynucleotide
gctgcttgttacgtttctgc

SEQ ID NO: 63 - DNA, 22 bp, Artificial Sequence
  Synthetic polynucleotide
cgcgaagtgcagtccaaaatcg

SEQ ID NO: 64 - DNA, 23 bp, Artificial Sequence
  Synthetic polynucleotide
gggctggttcttcttcaatgggg

SEQ ID NO: 65 - DNA, 22 bp, Artificial Sequence
  Synthetic polynucleotide
gaccgggagaatatctcagagt

SEQ ID NO: 66 - DNA, 21 bp, Artificial Sequence
  Synthetic polynucleotide
cattgaacaaccgatgcgtga

SEQ ID NO: 67 - DNA, 21 bp, Artificial Sequence
  Synthetic polynucleotide
cccacaagtcttgcagaaaca

SEQ ID NO: 68 - DNA, 23 bp, Artificial Sequence
  Synthetic polynucleotide
ctggctagtggtatatgtcacct

SEQ ID NO: 69 - DNA, 20 bp, Artificial Sequence
  Synthetic polynucleotide
atgttgtgggagatgtttgc

SEQ ID NO: 70 - DNA, 20 bp, Artificial Sequence
  Synthetic polynucleotide
gcagataagagctcagcctt

SEQ ID NO: 71 - DNA, 24 bp, Artificial Sequence
  Synthetic polynucleotide
ggactcatgaccacagtccatgcc

SEQ ID NO: 72 - DNA, 22 bp, Artificial Sequence
  Synthetic polynucleotide
tcagggatgaccttgcccacag

SEQ ID NO: 73 - DNA, 21 bp, Artificial Sequence
  Synthetic polynucleotide
gacgagtcaaaggtggaagac

SEQ ID NO: 74 - DNA, 22 bp, Artificial Sequence
  Synthetic polynucleotide
gattgtcatccgagctgtagtc

SEQ ID NO: 75 - DNA, 20 bp, Artificial Sequence
  Synthetic polynucleotide
ctgtcggagcaggacctaca

SEQ ID NO: 76 - DNA, 19 bp, Artificial Sequence
  Synthetic polynucleotide
gcgtccgttgatcttcagc

SEQ ID NO: 77 - DNA, 20 bp, Artificial Sequence
  Synthetic polynucleotide
agcttgggtgcctccttatt

SEQ ID NO: 78 - DNA, 20 bp, Artificial Sequence
  Synthetic polynucleotide
tgcttccctgaaagacatca

SEQ ID NO: 79 - DNA, 19 bp, Artificial Sequence
  Synthetic polynucleotide
caagtgtgcgtctgccttt

SEQ ID NO: 80 - DNA, 22 bp, Artificial Sequence
  Synthetic polynucleotide
cagctctttcttttcggctcta

SEQ ID NO: 81 - DNA, 26 bp, Artificial Sequence
  Synthetic polynucleotide
agattatatcaggttgtacgggatca

SEQ ID NO: 82 - DNA, 20 bp, Artificial Sequence
  Synthetic polynucleotide
acacagcggaaacactcgat

SEQ ID NO: 83 - DNA, 20 bp, Artificial Sequence
  Synthetic polynucleotide
agcctgtgtctttccatcgt

SEQ ID NO: 84 - DNA, 20 bp, Artificial Sequence
  Synthetic polynucleotide
gctgcccgaactaggtgtat

SEQ ID NO: 85 - DNA, 20 bp, Artificial Sequence
  Synthetic polynucleotide
aaagtgtcccctgcattctg

SEQ ID NO: 86 - DNA, 20 bp, Artificial Sequence
  Synthetic polynucleotide
cactccagggttccacatct

SEQ ID NO: 87 - DNA, 24 bp, Artificial Sequence
  Synthetic polynucleotide
gcaccttctgcctcagcaaccagc

SEQ ID NO: 88 - DNA, 21 bp, Artificial Sequence
  Synthetic polynucleotide
ggtcccccggtccctacacaa

SEQ ID NO: 89 - DNA, 20 bp, Artificial Sequence
  Synthetic polynucleotide
cacactcggtcagcaacatc

SEQ ID NO: 90 - DNA, 22 bp, Artificial Sequence
  Synthetic polynucleotide
ggtttctctagcccattcactg

SEQ ID NO: 91 - DNA, 20 bp, Artificial Sequence
  Synthetic polynucleotide
cgagggatgaacagtcctgg

SEQ ID NO: 92 - DNA, 21 bp, Artificial Sequence
  Synthetic polynucleotide
gtcatgtctgaggcgatgaag

SEQ ID NO: 93 - DNA, 21 bp, Artificial Sequence
  Synthetic polynucleotide
tacctgaggagtcacggaaaa

SEQ ID NO: 94 - DNA, 23 bp, Artificial Sequence
  Synthetic polynucleotide
ttcacagacatcgtagcacattt

SEQ ID NO: 95 - DNA, 20 bp, Artificial Sequence
  Synthetic polynucleotide
tgcaagagtgtgctagaggc

SEQ ID NO: 96 - DNA, 21 bp, Artificial Sequence
  Synthetic polynucleotide
acaaagcagtccacgaggtag

SEQ ID NO: 97 - DNA, 20 bp, Artificial Sequence
  Synthetic polynucleotide
tgacggacaagtacaggctg

SEQ ID NO: 98 - DNA, 21 bp, Artificial Sequence
  Synthetic polynucleotide
agggaagcgtgatgacaaaga

SEQ ID NO: 99 - DNA, 23 bp, Artificial Sequence
  Synthetic polynucleotide
aaggactggtactatacccacag

SEQ ID NO: 100 - DNA, 23 bp, Artificial Sequence
  Synthetic polynucleotide
tgtctgcttggtctttatcaacc

SEQ ID NO: 101 - DNA, 20 bp, Artificial Sequence
  Synthetic polynucleotide
ggggcaacaccttcaacctc

SEQ ID NO: 102 - DNA, 19 bp, Artificial Sequence
  Synthetic polynucleotide
ccaggcgaaactgaaaggc

SEQ ID NO: 103 - DNA, 22 bp, Artificial Sequence
  Synthetic polynucleotide
acccaagtgaaaaatgcgaatg

SEQ ID NO: 104 - DNA, 22 bp, Artificial Sequence
  Synthetic polynucleotide
cctccaaatcgaagtgagggtt

SEQ ID NO: 105 - DNA, 19 bp, Artificial Sequence
  Synthetic polynucleotide
actgccgtgacttcgacac

SEQ ID NO: 106 - DNA, 21 bp, Artificial Sequence
  Synthetic polynucleotide
gccttgccgtcttaaagttga

SEQ ID NO: 107 - DNA, 21 bp, Artificial Sequence
  Synthetic polynucleotide
cggatcttccaagagcaagtg

SEQ ID NO: 108 - DNA, 21 bp, Artificial Sequence
  Synthetic polynucleotide
acaggcatctgaggtgaacag

SEQ ID NO: 109 - DNA, 20 bp, Artificial Sequence
  Synthetic polynucleotide
ccaccgggaaggtgaatgtc

SEQ ID NO: 110 - DNA, 21 bp, Artificial Sequence
  Synthetic polynucleotide
ctgggcgcactttttgtagaa

SEQ ID NO: 111 - DNA, 22 bp, Artificial Sequence
  Synthetic polynucleotide
aactgcgagtggattgtttacg

SEQ ID NO: 112 - DNA, 23 bp, Artificial Sequence
  Synthetic polynucleotide
tctcgatttcaaagtgagggttg

SEQ ID NO: 113 - DNA, 21 bp, Artificial Sequence
  Synthetic polynucleotide
ggcgaactcgtatgaagatgc

SEQ ID NO: 114 - DNA, 22 bp, Artificial Sequence
  Synthetic polynucleotide
ctgggaaattatggttgctcct

SEQ ID NO: 115 - DNA, 20 bp, Artificial Sequence
  Synthetic polynucleotide
ctggaccgagtttgtggagg

SEQ ID NO: 116 - DNA, 23 bp, Artificial Sequence
  Synthetic polynucleotide
gtcacatagaagtagatgagccg

SEQ ID NO: 117 - DNA, 22 bp, Artificial Sequence
  Synthetic polynucleotide
aagccaggtattgcagttcttt

SEQ ID NO: 118 - DNA, 22 bp, Artificial Sequence
  Synthetic polynucleotide
gcatttgctgatgtctagcaca

SEQ ID NO: 119 - DNA, 21 bp, Artificial Sequence
  Synthetic polynucleotide
gacgtggatgcagctactcta

SEQ ID NO: 120 - DNA, 21 bp, Artificial Sequence
  Synthetic polynucleotide
agagattcaattctgcgctcc

SEQ ID NO: 121 - DNA, 20 bp, Artificial Sequence
  Synthetic polynucleotide
ccctagaggtcccctatggg

SEQ ID NO: 122 - DNA, 21 bp, Artificial Sequence
  Synthetic polynucleotide
tggcagaaaactcttcagcac

SEQ ID NO: 123 - DNA, 25 bp, Artificial Sequence
  Synthetic polynucleotide
cagtccaccaccagcatcttccagt

SEQ ID NO: 124 - DNA, 21 bp, Artificial Sequence
  Synthetic polynucleotide
agccaagactcgcggctacga

SEQ ID NO: 125 - DNA, 20 bp, Artificial Sequence
  Synthetic polynucleotide
cggctgcgcgtgtctaatcc

SEQ ID NO: 126 - DNA, 22 bp, Artificial Sequence
  Synthetic polynucleotide
ctgtgacctccagcagcttccg

SEQ ID NO: 127 - DNA, 19 bp, Artificial Sequence
  Synthetic polynucleotide
atgggtcaaggggcatcct

SEQ ID NO: 128 - DNA, 23 bp, Artificial Sequence
  Synthetic polynucleotide
ggcccaagattctttgtgtaagt

SEQ ID NO: 129 - protein, 558 aa, Homo sapiens
MRCALALSALLLLLSTPPLLPSSPSPSPSPSQNATQTTTDSSNKTAPTPASSVTIMATDTAQQSTVPTSKANEILASVKATTLGVSSDSPGTTTLAQQVSGPVNTTVARGGGSGNPTTTIESPKSTKSADTTTVATSTATAKPNTTSSQNGAEDTTNSGGKSSHSVTTDLTSTKAEHLTTPHPTSPLSPRQPTSTHPVATPTSSGHDHLMKISSSSSTVAIPGYTFTSPGMTTTLLETVFHHVSQAGLELLTSGDLPTLASQSAGITASSVISQRTQQTSSQMPASSTAPSSQETVQPTSPATALRTPTLPETMSSSPTAASTTHRYPKTPSPTVAHESNWAKCEDLETQTQSEKQLVLNLTGNTLCAGGASDEKLISLICRAVKATFNPAQDKCGIRLASVPGSQTVVVKEITIHTKLPAKDVYERLKDKWDELKEAGVSDMKLGDQGPPEEAEDRFSMPLIITIVCMASFLLLVAALYGCCHQRLSQRKDQQRLTEELQTVENGYHDNPTLEVMETSSEMQEKKVVSLNGELGDSWIVPLDNLTKDDLDEEEDTHL

SEQ ID NO: 130 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 131 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 132 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR